(19) United States
(12) Patent Application Publication
(10) Pub. No.: US 2024/0374657 A1
(43) Pub. Date: Nov. 14, 2024

(54) Inflammatory Respiratory Conditions and Treatments Thereof

(71) Applicant: Zephyrus Therapeutics, Inc., Needham, MA (US)

(72) Inventors: Laura ENSIGN, Towson, MD (US); James BURGESS, Stowe, VT (US); Thomas Gundelund RASMUSSEN, Wellesley, MA (US); Ulrich K. BINNE, Newton, MA (US); Johan E.T. VAN HYLCKAMA VLIEG, Belmont, MA (US)

(21) Appl. No.: 18/293,031

(22) PCT Filed: Jul. 29, 2022
(86) PCT No.: PCT/US2022/038882

§ 371 Date: Jan. 29, 2024

Related U.S. Application Data
(60) Provisional application No. 63/226,846, filed on Jul. 29, 2021.

(51) Int. Cl. A61K 35/741; A61K 35/24; A61K 45/06; A61P 11/00

(52) U.S. Cl. CPC A61K 35/741; A61K 35/24; A61K 45/06; A61P 11/00

(57) ABSTRACT

Provided are methods to treat upper respiratory tract inflammatory diseases and conditions including (chronic) sinusitis, (chronic) rhinitis, and/or (chronic) rhinosinusitis (with or without polyps), as well as nasal polyposis. Further provided are substantially complete sinonasal microbial compositions, methods of producing same, and medical kits comprising same.

### RELATED APPLICATIONS

This application claims the benefit of U.S. Provisional Patent Application 63/226,846, filed Jul. 29, 2021, the entire contents of which are incorporated herein by reference in its entirety.

### BACKGROUND

Sinusitis, rhinitis and rhinosinusitis are diseases associated with an inflammation of the upper airways, including the nose and paranasal sinuses (sinonasal cavities). Inflammation of the sinuses can cause mucosal edema and increased sinonasal secretions and obstruction of the sinuses and can be associated with microbial growth. Destruction of mucosal epithelial cells, imbalance of the host immune system (e.g., diminished host defenses), and unwanted growth of microbes in the sinonasal cavities (including the presence of microbial biofilms, e.g., lining the paranasal mucosa) and a subsequent state of chronic immuno-inflammatory activation may all play a role in the development and pathophysiology of chronic forms of the diseases.

Sinusitis, rhinitis and rhinosinusitis associated symptoms are varied and can include nasal congestion/obstruction, facial discomfort (pain/pressure, tenderness and swelling around the patient's eyes, cheeks, nose or forehead), nasal discharge, headache, hyposmia/anosmia, ear pain/pressure, aching in the upper jaw and teeth, cough, sore throat, fatigue, irritability, and nausea. These symptoms can significantly affect quality of life, sleep quality, and work productivity.

Sinusitis is classified as acute or chronic based on the duration of symptoms. Chronic forms of the diseases (chronic sinusitis, chronic rhinitis and chronic rhinosinusitis) represent a large portion of sinusitis cases and affect millions of people resulting in a large number of primary care and emergency room visits and associated health care and economic burden (e.g., loss of work days).

Chronic rhinosinusitis is frequently associated with asthma (a chronic inflammatory disease of the lower airways) and occasionally with aspirin sensitivity. Uncontrolled upper airway inflammation in the context of CRS is thought to be associated with lower airway T-helper-2 (Th2)-mediated inflammation and recalcitrant asthma, and rhinosinusitis appears to be linked to increased asthma severity and exacerbation rate. Th1/Th17 inflammatory pathways have also been suggested to be involved. CRS may also be associated with gastroesophageal reflux disease (GERD) and immunodeficiency. Chronic sinusitis, chronic rhinitis and chronic rhinosinusitis can precondition subjects to bronchitis and pneumonia, especially in the elderly.

The most common treatments for chronic sinusitis include steam or mist inhalation, antibiotics, corticosteroids and decongestants which can be successful in reducing discomfort (mucosal swelling) and relieving obstruction of the sinus ostium (drainage). However, chronic, and recurrent acute sinusitis, can be lifelong conditions and are often difficult to treat or resistant to medical therapy. Oral and intravenous administration of antibiotics, corticosteroids, or antifungals used to treat chronic sinusitis may lead to side effects (e.g., gastrointestinal), potentially resulting in premature discontinuation of the prescribed medication and other long-term, potentially detrimental health effects for the patient. Many chronic patients become candidates for (endoscopic) sinus surgeries. However, disease recurrence is reported to be frequent after surgery. There is a continued need for more effective treatment methods and therapeutic agents.

### SUMMARY OF THE INVENTION

Aspects relate to substantially complete sinonasal microbial compositions for use in a method of reducing symptoms or the severity of symptoms associated with chronic rhinosinusitis (CRS) in a recipient subject in need thereof, the method comprising administering an effective amount of the composition to sinonasal cavities of the recipient subject, thereby reducing symptoms or the severity of symptoms associated with CRS; wherein the composition is formulated into a dosage form; wherein the dosage form comprises an effective amount of the composition in one or more discrete units, wherein the effective amount is predetermined and effective in reducing the symptoms or the severity of symptoms associated with CRS; wherein said composition comprises sinonasal microbes of one or more of Actinobacteria, Bacteroidetes, Firmicutes or Proteobacteria, wherein the composition is obtainable by a method comprising:
a. Processing collected mucosal fluid comprising sinonasal microbes from sinonasal cavities of a healthy donor subject in a centralized processing facility,
b. Assessing the absence of one or more pathogens,
c. Assessing viability and/or quantity of the sinonasal microbes, and
d. Releasing the composition comprising the processed mucosal fluid for the aforementioned use if a predetermined level is obtained in step (b) and (c),
   wherein steps (b) and (c) can be performed prior to and/or after the processing in step (a) thereby obtaining the composition. For example, if step (b) and/or (c) is performed prior to processing, the absence of pathogens and/or the viability and/or quantity of sinonasal microbes of the collected mucosal fluid are assessed. If step (b) and/or (c) is performed after the processing step, the absence of pathogens and/or the viability and/or quantity of sinonasal microbes of the composition are assessed.

In embodiments, reducing the symptoms or the severity of symptoms associated with CRS comprises treating CRS.

In embodiments, the recipient subject has nasal or sinonasal polyps. In embodiments, the recipient subject has no nasal or sinonasal polyps. In embodiments, wherein the recipient subject exhibits refractory or recalcitrant rhinosinusitis. In one embodiment, the subject is diagnosed with refractory or recalcitrant CRS without polyps. In one embodiment, the subject is diagnosed with refractory or recalcitrant CRS with polyps. In embodiments, wherein the recipient subject exhibits one or more of: asthma, methicillin resistant Staphylococcus aureus (MRSA) infection, Samter's triad/polyposis (Aspirin Exacerbated Respiratory Disease (AERD)), and/or sinonasal sarcoidosis. In embodiments, the recipient subject exhibits one or more of gastroesophageal reflux disease (GERD), immunodeficiency, bronchitis and pneumonia.

Aspects relate to substantially complete sinonasal microbial compositions for use in a method of treating the sinonasal cavities of a recipient subject in need of treatment exhibiting an adverse condition affecting said sinonasal cavities, the method comprising administering an effective amount of the composition to sinonasal cavities of the recipient subject, thereby treating the adverse condition; wherein the composition is formulated into a dosage form; wherein the dosage form comprises an effective amount of the composition in one or more discrete units, wherein the effective amount is predetermined and effective in treating the adverse condition; wherein said composition comprises sinonasal microbes of one or more of Actinobacteria, Bacteroidetes, Firmicutes or Proteobacteria, wherein the composition is obtainable by a method comprising:
a. processing collected mucosal fluid comprising sinonasal microbes from sinonasal cavities of a healthy donor subject in a centralized processing facility,
b. assessing the absence of one or more pathogens,
c. assessing viability and/or quantity of the sinonasal microbes, and
d. releasing the composition comprising the processed mucosal fluid for the aforementioned use if a predetermined level is obtained in step (b) and (c),
   wherein steps (b) and (c) can be performed prior to and/or after the processing in step (a), thereby obtaining the composition.

Aspects relate to substantially complete sinonasal microbial compositions for use in a method of reducing the number of repeated sinus medical therapy and/or sinus surgery in a recipient subject in need of such treatment, the method comprising administering an effective amount of the composition to sinonasal cavities of the recipient subject, thereby reducing the number of repeated sinus medical therapy and/or sinus surgery; wherein the composition is formulated into a dosage form; wherein the dosage form comprises an effective amount of the composition in one or more discrete units, wherein the effective amount is predetermined and effective in reducing the number of repeated sinus medical therapy and/or sinus surgery; wherein said composition comprises sinonasal microbes of one or more of Actinobacteria, Bacteroidetes, Firmicutes or Proteobacteria, wherein the composition is obtainable by a method comprising:
a. processing collected mucosal fluid comprising sinonasal microbes from sinonasal cavities of a healthy donor subject in a centralized processing facility,
b. assessing the absence of one or more pathogens,
c. assessing viability and/or quantity of the sinonasal microbes, and
d. releasing the composition comprising the processed mucosal fluid for the aforementioned use if a predetermined level is obtained in step (b) and (c),
   wherein steps (b) and (c) can be performed prior to and/or after the processing in step (a), thereby obtaining the composition.

In embodiments, the recipient subject is diagnosed with sinusitis. In embodiments, the sinusitis is acute sinusitis, sub-acute sinusitis, or chronic sinusitis. In embodiments, the sinusitis is allergic sinusitis, or non-allergic sinusitis.

In embodiments, the recipient subject is diagnosed with rhinitis. In embodiments, the rhinitis is: allergic rhinitis, non-allergic rhinitis, allergic rhinitis rhinosinusitis, or non-allergic rhinosinusitis. In embodiments, the recipient subject is diagnosed with chronic sinusitis (CS) or chronic rhinosinusitis (CRS).

In embodiments, the recipient subject has nasal or sinonasal polyps. In embodiments, the recipient subject has no nasal or sinonasal polyps. In embodiments, the recipient subject exhibits refractory or recalcitrant sinusitis or rhinosinusitis.

In embodiments, the recipient subject exhibits one or more of: asthma, methicillin resistant Staphylococcus aureus (MRSA) infection, Samter's triad/polyposis (Aspirin Exacerbated Respiratory Disease (AERD)), and/or sinonasal sarcoidosis. In embodiments, the recipient subject exhibits one or more of gastroesophageal reflux disease (GERD), immunodeficiency, bronchitis and pneumonia.

In embodiments, the methods of obtaining the composition further comprise one, two, three, four or all of: (a) adding at least one pharmaceutically acceptable diluent, excipient or carrier; (b) adjusting the pH, osmolarity and/or viscosity of the mucosal fluid; (c) adding one or more cryoprotectants (e.g., for freezing) and/or one or more lyoprotectants (e.g., for drying); (d) formulating the processed mucosal fluid into a dosage form comprising a powder, a solid, a semi-solid, or a liquid; and (e) partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU). In embodiments, the method of obtaining the composition further comprises storing the composition for at least 24 hours or at least 48 hours prior to administration to the recipient subject.

In embodiments, for the methods provided herein, one or more (pharmaceutically acceptable) excipients or carriers are added to the mucosal fluid or the mucosal fluid is otherwise further processed. In embodiments, one or more of: pH, osmolarity and/or viscosity are adjusted, e.g., by adding acids, bases, buffers, and/or salts to the mucosal fluid. In embodiments, the composition is frozen or dried (e.g., spray dried/lyophilized) prior to administration to the recipient subject. In embodiments, the composition is not administered to the recipient subject as freshly harvested mucosal fluid, but is frozen or dried (e.g., lyophilized) prior to administration to the recipient subject. In some embodiments, the composition is stored in quarantine until the donor samples comprising the substantially complete sinonasal microbial composition and/or the donor subject have been assessed for the presence of pathogens, and/or transmittable diseases.

In embodiments, the dosage from comprises a composition that is frozen or dried (e.g., lyophilized). In embodiments, wherein the composition or dosage form is reconstituted or transitioned to a liquid or aerosolized form prior to administration to the recipient subject. In embodiments, administering to the recipient subject is performed using a syringe, catheter, nasal dropper, e.g., for nasal irrigation, spray, nebulizer, or aerosolizer, optionally a pump aerosol or a pulsating aerosol. In embodiments, the substantially complete sinonasal microbial composition is administered to a recipient subject using a syringe, catheter, or nasal dropper. In embodiments, the substantially complete sinonasal microbial composition is administered to a recipient subject as a spray. In embodiments, the substantially complete sinonasal microbial composition is administered to a recipient subject as a nebulizer. In embodiments, the substantially complete sinonasal microbial composition is administered to a recipient subject as an aerosol or aerosolizer, In embodiments, the substantially complete sinonasal microbial composition is administered to a recipient subject as a pump aerosol or a pulsating aerosol. In embodiments, the substantially complete sinonasal microbial composition is not administered to a recipient subject by an endoscopic administration route.

In embodiments, collecting mucosal fluid comprising sinonasal microbes from sinonasal cavities of a healthy donor subject comprises obtaining the mucosal fluid by rinsing the nasal cavities of the healthy donor subject with a pharmaceutically acceptable rinsing solution.

In embodiments, the collected mucosal fluid is collected from a healthy donor subject by lavage/rinsing of the sinonasal cavities. In embodiments, lavage/rinsing may be performed with 5 ml, 10 ml, 15 ml, 20 ml, or 25 ml or more of a suitable rinsing solution (e.g., saline).

In embodiments, processing the collected mucosal fluid comprises reducing the volume of the mucosal fluid, optionally by at least 25% or at least 50%. In embodiments, the volume of the mucosal fluid may be reduced by 75%, 90% or more. The volume of the mucosal fluid may be reduced to a suitable volume for administration into the sinonasal cavities, e.g., the volume is reduced to 0.5-10 ml, such as 1-10 ml, 2-8 ml, 3-7 ml. In some embodiments, the volume is reduced to less than or equal to about 1 ml, 2 ml, 3 ml, 4 ml, 5 ml, or less than or equal to about 10 ml, e.g., per donation from a healthy donor subject.

In embodiments, the composition further comprises one or more of: nasal mucus, nasal saline wash and nasal discharge, optionally, wherein the composition comprises sinonasal mucus (e.g., a brushing comprising mucus from the sinonasal cavities).

In embodiments, the healthy donor subject donates multiple mucosal fluid samples for administration to different recipient subjects. In embodiments, the composition is obtained from a donor that is not a family member of the recipient subject and/or is not known to the recipient subject.

In embodiments, the substantially complete sinonasal microbial composition comprises one or more of Bifidobacterium, Corynebacterium, Staphylococcus, Streptococcus, Dolosigranulum, Moraxella, Haemophilus, Moraxella, or Pseudomonas.

In embodiments, the substantially complete sinonasal microbial composition further comprises a pharmaceutically acceptable diluent, excipient or carrier. In embodiments, the substantially complete sinonasal microbial composition comprises one or more of a polymer, a carbon source, a mucoadhesive agent, a cryoprotectant, a lyoprotectant, a pH modifier and/or a buffering agent.

In embodiments, the substantially complete sinonasal microbial composition is administered nasally, trans-nasally, or to the sinuses.

In embodiments, the methods further comprising cleaning the sinonasal cavities of the recipient subject prior to administration of the substantially complete sinonasal microbial composition, wherein cleaning the sinonasal cavities comprises at least a partial removal of one or more of: debris/solids, mucus and/or microbes from at least a portion of the sinonasal cavities. In embodiments, cleaning the sinonasal cavities comprises at least a 1-log reduction of the sinonasal microbes comprised in the biofilm of at least a portion of the sinonasal cavities of the recipient subject. In embodiments, cleaning the sinonasal cavities comprises a 1-log reduction, 2-log reduction, or 3-log reduction from the initial quantity of microbes present in the biofilm in the sinonasal cavities of the recipient subject. In embodiments, cleaning the sinonasal cavities comprises one or more of: cleansing/rinsing, pressurized cleansing/rinsing, mechanical disruption of the biofilm (e.g., vibrating balloon), sinonasal scrubbing, salt washes, endoscopic/surgical removal, antimicrobial photodynamic therapy, antimicrobials (e.g., antibiotics), surfactants, mucolytics, salt washes (e.g., iodine) or a combination thereof.

In embodiments, the dosage form comprises an effective dose of sinonasal microbes derived from sinonasal cavities of one healthy donor subject (e.g., a mucosal fluid sample donor). In embodiments, the dosage form comprises a single or multiple effective doses of sinonasal microbes, wherein the effective dose of sinonasal microbes comprises about 10⁵ to 10¹² colony forming units (CFUs), e.g. about 10⁶-10¹² CFUs, about 10⁷-10¹² CFUs, about 10′-10¹² CFUs, about 10⁹-10¹² CFUs, about 10¹⁰-10¹² CFUs, or about 10¹¹-10¹² CFUs, and/or is effective in ameliorating the symptoms associated with (e.g., treating) acute sinusitis, rhinitis or rhinosinusitis or chronic sinusitis, rhinitis or rhinosinusitis. In embodiments, the effective dose of sinonasal microbes comprises at least 10⁵, 10⁶, 10⁷, 10⁸, 10⁹, 10¹⁰, 10¹¹, or 10¹² CFU.

In embodiments, the effective dose of sinonasal microbes is derived from the sinus microbiota (e.g., the microbial community preferentially residing in the sinus cavities). In embodiments, the effective dose of sinonasal microbes is derived from the nasal microbiota (e.g., the microbial community preferentially residing in the nasal cavities). In embodiments, the effective dose of sinonasal microbes is derived from both the sinus and nasal microbiota.

In embodiments, the healthy donor subject is selected by screening for the absence (e.g., local, e.g., within the sinonasal cavities, or serological, e.g., blood, plasma) of one or more microbial pathogens. In embodiments, the healthy donor subject is selected by screening for the absence of one or more transmittable diseases (e.g., influenza, tuberculosis).

In embodiments, administration of the substantially complete sinonasal microbial composition to the recipient subject reduces inflammation. In embodiments, the inflammation is local (e.g., the sinonasal cavities) or affecting larger areas of the body (e.g., upper airway inflammation). In embodiments, the reduction of inflammation is assessed by measuring one or more markers of inflammation (e.g., pro- and/or anti-inflammatory markers) in the subject, comprising one or more of: interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), interferons (e.g., IFN-γ), and/or Periostin, eosinophil cationic protein (ECP), P-glycoprotein (P-gp). In embodiments, the one or more markers of inflammation comprise one or more of IL-8, RANTES, and TGF-beta.

In embodiments, the reduction of symptoms or the severity of symptoms and progression of treatment is assessed by the improvement of one or more of: (a) Sino Nasal Outcome Test (e.g., SNOT-20 or SNOT-22) score; (b) nasal congestion/obstruction assessment, comprising one or more of: including anterior rhinorrhea (runny nose), posterior rhinorrhea (post nasal drip) and loss of sense of smell; (c) number of nightly awakenings; (d) visual analog score (VAS); (e) asthma control questionnaire (ACQ5) score; (f) nasal peak inspiratory flow (NPIF); (g) smell test (University of Pennsylvania Smell Identification Test (UPSIT)); (h) assessment of one or more physiological parameters (e.g., by nasal endoscopy or CT scan); (i) Lund-Mackay Score; (j) Modified Lund-Kennedy score; and (k) volumetric measurements of at least one part of the sinonasal cavities. In embodiments, the improvement of the score or test results of one or more of (a)-(k) are maintained for at least 1 week, 2 weeks, 3 weeks, 1 month, 2 months, 4 months, 6 months, 8 months, 10 months, 1 year, 2 years, 3 years, 5 years, 10 years, or more than 10 years.

In embodiments, the improvement of the score or test results of one or more of (a)-(k) are maintained for at least 2 months, 4 months, 6 months, 8 months, 10 months, 1 year, 2 years, 3 years, 5 years, 10 years, or more than 10 years. In embodiments, the improvement of the score or test results of one or more of (a)-(k) are maintained for at least 1 year, 2 years, 3 years, 5 years, 10 years, or more than 10 years. In embodiments, the treatment progression is determined or monitored by a reduction of the SNOT-22 score. In embodiments, the SNOT-22 score is reduced by at least 9 points, such as at least 10, 15, 20, 25, 30 or more SNOT-22 points from baseline (e.g., prior to administration of the composition). In embodiments, for the methods provided herein, treatment progression is determined or monitored by a reduction of the SNOT-22 score. In embodiments, a reduction of the SNOT-22 score by at least 9 points is considered to be clinically relevant.

In embodiments, the methods further comprise administering to the recipient subject a co-therapeutic treatment comprising one or more of: antimicrobial (e.g., antibiotic, anti-fungal, anti-viral), anti-inflammatory agent/corticosteroid, anti-septic (e.g., iodine), mucolytic, antihistamine medication, decongestant, vasoconstrictor, or a chelating agent medication, or a biofilm-disrupting treatment (e.g. a biofilm disrupting wash, mechanical disruption (e.g. vibrating balloon), cleansing (e.g., salt washes (e.g., iodine)), sinonasal scrubbing, endoscopic removal, and the use of surfactants, mucolytics, etc., as well as sterilization techniques that primarily kill or inhibit the growth of microbes, utilizing, e.g., antimicrobials and antimicrobial photodynamic therapy, or any combination thereof). In embodiments, the co-therapeutic treatment is an antimicrobial comprising amoxicillin, other penicillins, cephalosporins, amoxicillin/clavulanate potassium, clarithromycin, beta-lactam, a cephalosporin, a lincosamide, a macrolide, a tetracycline, a sulfa drug (e.g., compound), mupirocin, oxacillin, flucloxacillin, cefazolin, cephalothin, cephalexin, erythromycin, doxycycline, or minocycline.

In embodiments, the sinonasal microbes (e.g., bacteria) comprised in the complete sinonasal microbial composition are capable of stably engrafting (e.g., colonizing) the sinonasal niche of the subject for a time period of at least 1 month, 2, 3, 4, 5, 6, 7, 8, 9, 10, 11, or more than 12 months.

In embodiments, the composition is administered to the subject daily or twice daily. In embodiments, the composition is administered for at least two consecutive days, three consecutive days, four consecutive days, or five consecutive days.

In embodiments, the recipient subject prior to treatment displays one or more symptoms selected from the group consisting of nasal congestion/obstruction, facial discomfort/pain or pressure, tenderness and swelling around the patient's eyes, cheeks, nose or forehead, nasal discharge, headache, hyposmia, anosmia, ear pain/pressure, aching in the upper jaw and teeth, cough, sore throat, fatigue, irritability, and nausea, and further wherein the severity or occurrence of the one or more symptoms are reduced after the administration of the composition. In embodiments, the severity or occurrence of the one or more symptoms are reduced after the administration of the composition for at least 3 months, 6, months, 9 months, 12 months, 18 months, 24 months, or 36 months.

Aspects relate to methods of producing a substantially complete sinonasal microbial composition in a centralized processing facility comprising:
a. receiving and processing collected mucosal fluid comprising sinonasal microbes from sinonasal cavities of a healthy donor subject,
b. assessing the absence of one or more pathogens,
c. assessing viability and/or quantity of the sinonasal microbes, and
d. releasing a composition comprising the processed mucosal fluid if a predetermined level is obtained in step (b) and (c), optionally, discarding the composition if a predetermined level in step (b) and (c) is not obtained,
   wherein steps (b) and (c) can be performed prior to (e.g., with the mucosal fluid sample) and/or after the processing in step (a) (e.g., with the composition), e.g., by removing a portion of the mucosal fluid sample or composition for testing (e.g., nucleic acid sequencing), thereby obtaining the composition. In embodiments, the composition is a pharmaceutical composition.

In embodiments, the methods further comprise one, two, three, four, five or all of: (a) adding at least one pharmaceutically acceptable diluent, excipient or carrier (e.g., to create a diluted mucosal fluid sample); (b) adjusting the pH, osmolarity and/or viscosity of the mucosal fluid; (c) adding one or more cryoprotectants (e.g., for freezing) and/or one or more lyoprotectants (e.g., for drying); (d) formulating the processed mucosal fluid into a dosage form comprising a powder, a solid, a semi-solid, or a liquid; (e) partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU); and (f) preserving the mucosal fluid in a container, optionally for storage (e.g., refrigerated, frozen or dried).

In embodiments, the methods further comprise one or more of (a) storing the refrigerated, frozen or dried mucosal fluid sample or processed composition under quarantine, (b) holding the refrigerated, frozen or dried mucosal fluid sample or processed composition under quarantine until any completion of any combination of (i) testing the donor to exclude the substantial presence of one or more transmissible pathogens, (ii) confirming the composition and viability of the microbes comprised in the mucosal fluid sample, or (iii) further confirming the health of the donor by a plurality of post-screening tests; (c) standardizing the cell count and/or the quantity or concentration of the sinonasal microbes comprised in the composition, optionally by adding an inert filler; and (d) releasing the refrigerated, frozen or dried mucosal fluid sample or processed composition from quarantine to define the substantially complete sinonasal microbial composition.

In embodiments, the processing of the mucosal fluid, e.g., by addition of a cryoprotectant or lyoprotectant, and lyophilizing or (spray) drying, allows to maintain the viability of the sinonasal microbes contained therein for longer periods of time than if the substantially complete sinonasal microbial composition would be stored unprocessed. The processing further allows to provide a standardized concentration of the sinonasal microbes (e.g., a minimum level of sinonasal microbes, as assessed, e.g., by CFU counting), to ensure comparable doses for use in treatment. Moreover, the processing, in some embodiments, prolongs the shelf-life and stability of the composition, which allows the isolated mucosal fluid from one or more collected donations from one healthy donor subject to be quantified and combined. In some embodiments, the mucosal fluid comprising the sinonasal microbes is obtained from one healthy donor subject. In embodiments, the mucosal fluid obtained from multiple healthy donor subjects is processed separately and maintained and separately stored and not pooled.

The viability of the sinonasal microbes comprised in the substantially complete sinonasal microbial composition may be assessed by methods of the art, e.g., live/dead bacterial viability tests, determining cell proliferation, metabolic activity, PCR of microbe components or measuring cell death of the microbes in the sample. The quantity of the microbes comprised in a substantially complete sinonasal microbial composition can be ascertained by methods known in the art, and include, but are not limited to serial dilution and determining CFU/mL, determining the optical density, plate counting, turbidimetric analysis or under a counting chamber. In some embodiments, the substantially complete sinonasal microbial composition maintains or preserves at least 10%, 20%, 30%, 40%, 50%, 60%, 70% or a greater degree of viability of the microbes comprised in the donor subject mucosal fluid

In embodiments, the substantially complete sinonasal microbial composition maintains or preserves at least 50%, 55%, 60%, 65%, 70%, 75%, 80%, 85%, 90%, 92%, 95%, 97%, 98%, or at least 99% of the constituents of the microbiome comprised in the donor subject mucosal fluid. In embodiments, the methods further comprise the substantially complete sinonasal microbial composition maintains or preserves at least 10%, 20%, 30%, 40%, 50%, 60%, 70% or a greater degree of viability of the microbes comprised in the donor subject mucosal fluid.

In some embodiments of the methods described herein, if one or more predetermined level(s) is not met, e.g. the substantially complete sinonasal microbial composition comprises pathogens above the predetermined level (e.g., a maximum level) and/or does not comprise a predetermined level of viability or quantity of sinonasal microbes (e.g., a minimum level), the substantially complete sinonasal microbial composition is discarded, e.g., not released for one or more of the uses described herein (not released for use in subjects in the methods described herein). In some embodiments, the substantially complete sinonasal microbial compositions satisfies the one or more predetermined level(s). In embodiments, the substantially complete sinonasal microbial compositions may satisfy the predetermined level for pathogens, e.g., a substantial absence thereof but does not satisfy the predetermined (e.g., minimum level) of sufficient quantity or viability of sinonasal microbes. In such cases, two, three, four or more substantially complete sinonasal microbial compositions isolated from the same donor subject may be pooled to obtain a substantially complete sinonasal microbial composition that also meets the predetermined level for viability and/or quantity of sinonasal microbes.

In embodiments, the methods further comprise storing the composition for at least 24 hours or at least 48 hours prior to releasing the composition for use.

In embodiments, the composition is frozen or dried (e.g., lyophilized).

In embodiments, the methods further comprise collecting mucosal fluid comprising sinonasal microbes from sinonasal cavities of a healthy donor subject comprises obtaining the mucosal fluid by rinsing of the nasal cavities of the healthy donor subject with a pharmaceutically acceptable rinsing solution.

In embodiments, processing the collected mucosal fluid comprises reducing the volume of the mucosal fluid, optionally by at least 25% or at least 50%.

In embodiments, the composition further comprises one or more of: nasal mucus, nasal saline wash and nasal discharge, optionally, wherein the composition comprises sinonasal mucus (e.g., a brushing comprising mucus from the sinonasal cavities).

In embodiments, the healthy donor subject donates multiple mucosal fluid samples.

In embodiments, the composition further comprises a pharmaceutically acceptable diluent, excipient or carrier. In embodiments, the composition comprises one or more of a polymer, a carbon source, a mucoadhesive agent, a cryoprotectant, a lyoprotectant, a pH modifier and/or a buffering agent.

In embodiments, the sinonasal microbes are constituents of the sinus microbiota (e.g., the microbial community preferentially residing in the sinus cavities). In embodiments, the sinonasal microbes are constituents of the nasal microbiota (e.g., the microbial community preferentially residing in the nasal cavities). In embodiments, the sinonasal microbes are constituents of both the sinus microbiota and the nasal microbiota.

In embodiments, the healthy donor subject is selected by screening for the absence (e.g., local, e.g., within the sinonasal cavities, or serological, e.g., blood, plasma) of one or more microbial pathogens. In embodiments, the healthy donor subject is selected by screening for the absence of one or more transmittable diseases (e.g., influenza, tuberculosis). In embodiments, the one or more microbial pathogens or transmittable diseases comprise methicillin-resistant Staphylococcus aureus (MRSA), sexually transmissible diseases, e.g., gonorrhea, HIV, syphilis; orally or contact transmissible diseases, e.g., hepatitis, influenza, tuberculosis, measles, and other respiratory pathogens, such as, e.g., rhinovirus, respiratory syncytial virus, and other respiratory viruses, e.g., (severe acute respiratory syndrome) coronaviruses, e.g., SARS-CoV, SARS-CoV-2.

In embodiments, the complete sinonasal microbial composition is not obtained by selective culturing (e.g., under culturing conditions that favor specific taxa, e.g., using antimicrobials, specific nutrients or nutrient depletion, temperature, oxygen levels, etc.).

In embodiments, the complete sinonasal microbial composition comprises one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas. In embodiments, the substantially complete sinonasal microbial composition comprises one or more of Bifidobacterium, Corynebacterium, Staphylococcus, Streptococcus, Dolosigranulum, and Moraxella. In embodiments, the substantially complete sinonasal microbial composition comprises one or more of Corynebacterium, Staphylococcus, Streptococcus, Haemophilus, Moraxella, and Pseudomonas.

Aspects relate to isolated substantially complete sinonasal microbial compositions produced or obtainable by the method described herein. In embodiments, the composition is a pharmaceutical composition. In embodiments, the substantially complete sinonasal microbial composition comprises one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas. In embodiments, the substantially complete sinonasal microbial composition comprises one or more of Bifidobacterium, Corynebacterium, Staphylococcus, Streptococcus, Dolosigranulum, Moraxella, Haemophilus, Moraxella, Pseudomonas, Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellagioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus or Lentilactobacillus. In embodiments, the substantially complete sinonasal microbial composition comprises sinonasal microbes capable of colonizing the sinonasal cavities of a recipient subject for a time period of at least 1 month, 2 months, 3 months, 4 months, 5 months, 6 months or longer. In embodiments, the substantially complete sinonasal microbial composition further comprises at least one pharmaceutically acceptable carrier, diluent or excipient.

Aspects relate to dosage forms formulated for sinonasal administration comprising a substantially complete sinonasal microbial composition described herein, wherein the substantially complete sinonasal microbial composition can be produced or is obtainable by the methods described herein. In embodiments, the dosage form formulated for sinonasal administration comprises a substantially complete sinonasal microbial composition described herein. In embodiments, the dosage form comprises a substantially complete sinonasal microbial composition that is frozen or dried (e.g., lyophilized). In embodiments, the dosage form is suitable for sinonasal administration using a syringe, catheter, nasal dropper, e.g., for nasal irrigation, spray, nebulizer, or aerosolizer, optionally a pump aerosol or a pulsating aerosol. In embodiments, the dosage form is an aqueous solution/liquid suspension (e.g., a saline solution), spray, (dry) powder or aerosol.

Aspects also relate to composition (e.g., pharmaceutical compositions) comprising a substantially complete sinonasal microbial composition described herein.

Aspects further relate to medical kit comprising: (a) an applicator configured for delivery of the substantially complete sinonasal microbial composition described herein to the human sinonasal cavities, wherein the composition is optionally a fluid, aerosol, mist or dry powder; and (b) the substantially complete sinonasal microbial composition described herein or a composition comprising the same, described herein.

Aspects relate to methods of reducing symptoms or the severity of symptoms associated with chronic rhinosinusitis (CRS) in a subject in need thereof comprising administering an effective amount of a substantially complete sinonasal microbial composition to sinonasal cavities of the subject thereby reducing symptoms or the severity of symptoms associated with CRS, wherein said composition comprises:
a. isolated mucosal fluid comprising sinonasal microbes;
b. a predetermined quantity of viable sinonasal microbes,
c. does not comprise nasal pathogens above a predetermined level,
   wherein the sinonasal microbes comprise one or more of Actinobacteria, Bacteroidetes, Firmicutes or Proteobacteria.

In embodiments of the methods described herein, reducing the symptoms or the severity of symptoms associated with CRS comprises treating CRS. In some embodiments, the subject has nasal or sinonasal polyps. In other embodiments, the subject has no nasal or sinonasal polyps. In embodiments, the subject exhibits refractory or recalcitrant rhinosinusitis. In embodiments, the subject exhibits one or more of: asthma, methicillin resistant Staphylococcus aureus (MRSA) infection, Samter's triad/polyposis (Aspirin Exacerbated Respiratory Disease (AERD)), and/or sinonasal sarcoidosis. In some embodiments, the subject exhibits one or more of gastroesophageal reflux disease (GERD), immunodeficiency, bronchitis and pneumonia.

Aspects relate to methods to treat the sinonasal cavities of a subject in need of treatment exhibiting an adverse condition affecting said sinonasal cavities, the method comprising administering an effective amount of a substantially complete sinonasal microbial composition to sinonasal cavities of the subject, thereby treating the adverse condition, wherein said composition comprises:
a. isolated mucosal fluid comprising sinonasal microbes;
b. a predetermined quantity of viable sinonasal microbes,
c. does not comprise nasal pathogens above a predetermined level,
   wherein the sinonasal microbes comprise one or more of Actinobacteria, Bacteroidetes, Firmicutes or Proteobacteria.

Aspects relate to methods of reducing the number of repeated sinus medical therapy and/or sinus surgery in a subject in need of such treatment, the method comprising administering an effective amount of a substantially complete sinonasal microbial composition to sinonasal cavities of the subject, thereby reducing the number of repeated sinus medical therapy and/or sinus surgery; wherein said composition comprises:
a. isolated mucosal fluid comprising sinonasal microbes;
b. a predetermined quantity of viable sinonasal microbes,
c. does not comprise nasal pathogens above a predetermined level,
   wherein the sinonasal microbes comprise one or more of Actinobacteria, Bacteroidetes, Firmicutes or Proteobacteria.

In embodiments of the methods described herein, the subject is diagnosed with sinusitis. In embodiments, the sinusitis is acute sinusitis, sub-acute sinusitis, or chronic sinusitis. In embodiments, the sinusitis is allergic sinusitis, or non-allergic sinusitis.

In embodiments of the methods described herein, the subject is diagnosed with rhinitis. In embodiments, the rhinitis is allergic rhinitis, non-allergic rhinitis, allergic rhinitis rhinosinusitis, or non-allergic rhinosinusitis.

In embodiments of the methods described herein, the subject is diagnosed with chronic sinusitis (CS) or chronic rhinosinusitis (CRS).

In some embodiments, the subject has nasal or sinonasal polyps. In other embodiments, the subject has no nasal or sinonasal polyps. In embodiments, the subject exhibits refractory or recalcitrant sinusitis or rhinosinusitis. In embodiments, the subject exhibits one or more of asthma, methicillin resistant Staphylococcus aureus (MRSA) infection, Samter's triad/polyposis (Aspirin Exacerbated Respiratory Disease (AERD)), and/or sinonasal sarcoidosis. In embodiments, the subject exhibits one or more of gastroesophageal reflux disease (GERD), immunodeficiency, bronchitis and pneumonia.

In embodiments of the methods described herein, the substantially complete sinonasal microbial composition is administered to the subject using a syringe, catheter, nasal dropper, e.g., for nasal irrigation, spray, nebulizer, or aerosolizer, optionally a pump aerosol or a pulsating aerosol. In embodiments, the substantially complete sinonasal microbial composition is administered nasally, trans-nasally, or to the sinuses.

In embodiments of method described herein further comprise cleaning the sinonasal cavities of the subject prior to administration of the substantially complete sinonasal microbial composition, wherein cleaning the sinonasal cavities comprises at least a partial removal of one or more of: debris/solids, mucus and/or microbes from at least a portion of the sinonasal cavities. In embodiments, cleaning the sinonasal cavities comprises at least a 1-log reduction of the sinonasal microbes comprised in the biofilm of at least a portion of the sinonasal cavities of the subject. In embodiments, cleaning the sinonasal cavities comprises a 1-log reduction, 2-log reduction, or 3-log reduction from the initial quantity of microbes present in the biofilm in the sinonasal cavities of the subject. In embodiments, cleaning the sinonasal cavities comprises one or more of cleansing/rinsing, pressurized cleansing/rinsing, mechanical disruption of the biofilm (e.g. vibrating balloon), sinonasal scrubbing, salt washes, endoscopic/surgical removal, antimicrobial photodynamic therapy, antimicrobials (e.g., antibiotics), surfactants, mucolytics, salt washes (e.g., iodine) or a combination thereof.

In embodiments of the methods described herein, administration of the substantially complete sinonasal microbial composition to the subject reduces inflammation. In embodiments, the inflammation is local (e.g., the sinonasal cavities) or affecting larger areas of the body (e.g., upper airway inflammation). In embodiments, the reduction of inflammation is assessed by measuring one or more markers of inflammation (e.g., pro- and/or anti-inflammatory markers) in the subject, comprising one or more of: interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), interferons (e.g., IFN-γ), and/or Periostin, eosinophil cationic protein (ECP), P-glycoprotein (P-gp). In embodiments, one or more markers of inflammation comprise one or more of IL-8, RANTES, and TGF-beta.

In embodiments of the methods described herein, the reduction of symptoms or the severity of symptoms and progression of treatment is assessed by the improvement of one or more of: (a) Sino Nasal Outcome Test (e.g., SNOT-20 or SNOT-22) score; (b) nasal congestion/obstruction assessment, comprising one or more of: including anterior rhinorrhea (runny nose), posterior rhinorrhea (post nasal drip) and loss of sense of smell; (c) number of nightly awakenings; (d) visual analog score (VAS); (e) asthma control questionnaire (ACQ5) score; (f) nasal peak inspiratory flow (NPIF); (g) smell test (University of Pennsylvania Smell Identification Test (UPSIT)); (h) assessment of one or more physiological parameters (e.g., by nasal endoscopy or CT scan); (i) Lund-Mackay Score; (j) Modified Lund-Kennedy score; and (k) volumetric measurements of at least one part of the sinonasal cavities. In embodiments, the improvement of the score or test results of one or more of (a)-(k) are maintained for at least 1 week, 2 weeks, 3 weeks, 1 month, 2 months, 4 months, 6 months, 8 months, 10 months, 1 year, 2 years, 3 years, 5 years, 10 years, or more than 10 years. In embodiments, the improvement of the score or test results of one or more of (a)-(k) are maintained for at least 2 months, 4 months, 6 months, 8 months, 10 months, 1 year, 2 years, 3 years, 5 years, 10 years, or more than 10 years. In embodiments, the improvement of the score or test results of one or more of (a)-(k) are maintained for at least 1 year, 2 years, 3 years, 5 years, 10 years, or more than 10 years. In embodiments, the treatment progression is determined or monitored by a reduction of the SNOT-22 score. In embodiments, the SNOT-22 score is reduced by at least 9 points, such as at least 10, 15, 20, 25, 30 or more SNOT-22 points from baseline (e.g., prior to administration of the composition).

The methods described herein may further comprise administering to the subject a co-therapeutic treatment comprising one or more of: antimicrobial (e.g., antibiotic, anti-fungal, anti-viral), anti-inflammatory agent/corticosteroid, anti-septic (e.g., iodine), mucolytic, antihistamine medication, decongestant, vasoconstrictor, or a chelating agent medication, or a biofilm-disrupting treatment (e.g. a biofilm disrupting wash, mechanical disruption (e.g. vibrating balloon), cleansing (e.g., salt washes (e.g., iodine)), sinonasal scrubbing, endoscopic removal, and the use of surfactants, mucolytics, etc., as well as sterilization techniques that primarily kill or inhibit the growth of microbes, utilizing, e.g., antimicrobials and antimicrobial photodynamic therapy, or any combination thereof). In embodiments, the co-therapeutic treatment is an antimicrobial comprising amoxicillin, other penicillins, cephalosporins, amoxicillin/clavulanate potassium, clarithromycin, beta-lactam, a cephalosporin, a lincosamide, a macrolide, a tetracycline, a sulfa drug (e.g., compound), mupirocin, oxacillin, flucloxacillin, cefazolin, cephalothin, cephalexin, erythromycin, doxycycline, or minocycline.

In embodiments of the methods described herein, the sinonasal microbes (e.g., bacteria) comprised in the complete sinonasal microbial composition are capable of stably engrafting (e.g., colonizing) the sinonasal niche of the subject for a time period of at least 1 month, 2, 3, 4, 5, 6, 7, 8, 9, 10, 11, or more than 12 months.

In embodiments of the methods described herein, the composition is administered to the subject daily or twice daily. In embodiments, the composition is administered for at least two consecutive days, three consecutive days, four consecutive days, or five consecutive days.

In embodiments of the methods described herein, the subject prior to treatment displays one or more symptoms selected from the group consisting of nasal congestion/obstruction, facial discomfort/pain or pressure, tenderness and swelling around the patient's eyes, cheeks, nose or forehead, nasal discharge, headache, hyposmia, anosmia, ear pain/pressure, aching in the upper jaw and teeth, cough, sore throat, fatigue, irritability, and nausea, and further wherein the severity or occurrence of the one or more symptoms are reduced after the administration of the composition. In embodiments, the severity or occurrence of the one or more symptoms are reduced after the administration of the composition for at least 3 months, 6, months, 9 months, 12 months, 18 months, 24 months, or 36 months.

In embodiments of the methods described herein, the substantially complete sinonasal microbial composition further comprises at least one pharmaceutically acceptable diluent, excipient or carrier. In embodiments, the composition further comprises one or more cryoprotectants (e.g., for freezing). In embodiments, the composition further comprises one or more lyoprotectants (e.g., for drying).

In embodiments of the methods described herein, the substantially complete sinonasal microbial composition is in powder, solid, semi-solid, or liquid form. In embodiments, the composition is frozen or dried (e.g., lyophilized), optionally wherein the composition is reconstituted or transitioned to a liquid or aerosolized form prior to administration to the subject.

In embodiments of the methods described herein, the substantially complete sinonasal microbial composition further comprises an effective dose of sinonasal microbes of about 10³ to 10¹⁵ colony forming units (CFU) or 10⁵ to 10¹² colony forming units (CFU).

In embodiments of the methods described herein, the substantially complete sinonasal microbial composition comprises mucosal fluid comprising sinonasal microbes derived from the nasal cavities of the healthy donor subject.

In embodiments of the methods described herein, the substantially complete sinonasal microbial composition the composition further comprises one or more of: nasal mucus, nasal saline wash and nasal discharge, optionally, wherein the composition comprises sinonasal mucus (e.g., a brushing comprising mucus from the sinonasal cavities).

In embodiments of the methods described herein, the mucosal fluid comprised in the composition is obtained from a healthy donor that is not a family member of the subject and/or is not known to the subject. In embodiments of the methods described herein, the mucosal fluid comprised in the composition is obtained from a healthy donor that donates multiple mucosal fluid samples for administration to different subjects.

In embodiments of the methods described herein, the substantially complete sinonasal microbial composition comprises one or more of Bifidobacterium, Corynebacterium, Staphylococcus, Streptococcus, Dolosigranulum, Moraxella, Haemophilus, Moraxella, or Pseudomonas.

In embodiments of the methods described herein, the substantially complete sinonasal microbial composition further comprises a pharmaceutically acceptable diluent, excipient or carrier. In embodiments, the composition comprises one or more of a polymer, a carbon source, a mucoadhesive agent, a cryoprotectant, a lyoprotectant, a pH modifier and/or a buffering agent.

In embodiments of the methods described herein, the effective dose of sinonasal microbes comprises about 10⁵ to 10¹² colony forming units (CFUs), e.g. about 10⁶-10¹² CFUs, about 10⁷-10¹² CFUs, about 10′-10¹² CFUs, about 10⁹-10¹² CFUs, about 10¹⁰-10¹² CFUs, or about 10¹¹-10¹² CFUs, and/or is effective in ameliorating the symptoms associated with (e.g., treating) acute sinusitis, rhinitis or rhinosinusitis or chronic sinusitis, rhinitis or rhinosinusitis. In embodiments, the effective dose of sinonasal microbes comprises at least 10⁵, 10⁶, 10⁷, 10⁸, 10⁹, 10¹⁰, 10¹¹, or 10¹² CFU.

In embodiments of the methods described herein, the sinonasal microbes are derived from the sinus microbiota (e.g., the microbial community preferentially residing in the sinus cavities). In embodiments, the sinonasal microbes are derived from the nasal microbiota (e.g., the microbial community preferentially residing in the nasal cavities). In embodiments, the sinonasal microbes are derived from both the sinus and nasal microbiota. In embodiments, the substantially complete sinonasal microbial composition is substantially free of one or more microbial pathogens.

In embodiments of the methods described herein, the substantially complete sinonasal microbial composition is a pharmaceutical composition comprising a pharmaceutically acceptable excipient, diluent or carrier.

In embodiments of the methods described herein, the substantially complete sinonasal microbial composition is formulated as a dosage form suitable for sinonasal administration.

In embodiments of the methods described herein, the substantially complete sinonasal microbial composition has a predetermined pH. In embodiments, the composition has a predetermined osmolarity. In embodiments, the composition has a predetermined viscosity.

Aspects relate to an isolated substantially complete sinonasal microbial composition, wherein said composition comprises:
a. isolated mucosal fluid comprising sinonasal microbes;
b. a predetermined quantity of viable sinonasal microbes,
c. does not comprise one or more nasal pathogen above a predetermined level,
   wherein the sinonasal microbes comprise one or more of Actinobacteria, Bacteroidetes, Firmicutes or Proteobacteria.

In embodiments, the substantially complete sinonasal microbial composition is a pharmaceutical composition comprising a pharmaceutically acceptable excipient, diluent or carrier.

In embodiments, the substantially complete sinonasal microbial composition is formulated as a dosage form suitable for sinonasal administration.

In embodiments, the substantially complete sinonasal microbial composition is substantially free of pathogens (e.g., nasal pathogens), e.g., an absence of pathogens is determined. In embodiments, the substantially complete sinonasal microbial composition comprises less than 30%, 20%, 15%, 10%, 5%, 4%, 3%, 2%, 1%, 0.5% or less than 0.1% pathogens (e.g., bacterial pathogens) of the total amount, number or (relative) abundance of microbes (e.g., bacteria) as determined by a suitable method, the predetermined level. In embodiments, the substantially complete sinonasal microbial composition comprises less than 5%, 4%, 3%, 2%, 1%, or less than 0.5% pathogens of the total amount, number or (relative) abundance of microbes (e.g., bacteria) as determined by a suitable method, the predetermined level. In embodiments, the substantially complete sinonasal microbial composition is substantially free of one or more microbial pathogens. In embodiments of the methods described herein, the substantially complete sinonasal microbial composition is released if one or more predetermined level is met.

In embodiments, the substantially complete sinonasal microbial composition has a predetermined pH. In embodiments, the composition has a predetermined osmolarity. In embodiments, the composition has a predetermined viscosity.

In embodiments, the substantially complete sinonasal microbial composition further comprises at least one pharmaceutically acceptable diluent, excipient or carrier. In embodiments, the composition further comprises one or more cryoprotectants (e.g., for freezing). In embodiments, the composition further comprises one or more lyoprotectants (e.g., for drying).

In embodiments, the substantially complete sinonasal microbial composition is in powder, solid, semi-solid, or liquid form. In embodiments, the composition is frozen or dried (e.g., lyophilized), optionally wherein the composition is reconstituted or transitioned to a liquid or aerosolized form prior to administration to the subject.

In embodiments, the substantially complete sinonasal microbial composition comprises sinonasal microbes of a predetermined level of about 10³ to 10¹⁵ colony forming units (CFU) or 10⁵ to 10¹² colony forming units (CFU).

In embodiments, the substantially complete sinonasal microbial composition comprises mucosal fluid comprising sinonasal microbes isolated from the (sino-)nasal cavities of the healthy donor subject.

In embodiments, the substantially complete sinonasal microbial composition the composition further comprises one or more of: nasal mucus, nasal saline wash and nasal discharge, optionally, wherein the composition comprises sinonasal mucus (e.g., a brushing comprising mucus from the sinonasal cavities).

In embodiments, the substantially complete sinonasal microbial composition comprises one or more of Bifidobacterium, Corynebacterium, Staphylococcus, Streptococcus, Dolosigranulum, Moraxella, Haemophilus, Moraxella, or Pseudomonas.

In embodiments, the substantially complete sinonasal microbial composition further comprises a pharmaceutically acceptable diluent, excipient or carrier. In embodiments, the composition comprises one or more of a polymer, a carbon source, a mucoadhesive agent, a cryoprotectant, a lyoprotectant, a pH modifier and/or a buffering agent.

In embodiments, the substantially complete sinonasal microbial composition comprises sinonasal microbes of a predetermined level of about 10⁵ to 10¹² colony forming units (CFUs), e.g. about 10⁶-10¹² CFUs, about 10⁷-10¹² CFUs, about 10⁸-10¹² CFUs, about 10⁹-10¹² CFUs, about 10¹⁰-10¹² CFUs, or about 10¹¹-10¹² CFUs. In embodiments, the substantially complete sinonasal microbial composition comprises sinonasal microbes of a predetermined level of at least 10⁵, 10⁶, 10⁷, 10⁸, 10⁹, 10¹⁰, 10¹¹, or 10¹² CFU.

In embodiments, the sinonasal microbes are derived from the sinus microbiota (e.g., the microbial community preferentially residing in the sinus cavities). In embodiments, the sinonasal microbes are derived from the nasal microbiota (e.g., the microbial community preferentially residing in the nasal cavities). In embodiments, the sinonasal microbes are derived from both the sinus and nasal microbiota.

In embodiments, the substantially complete sinonasal microbial composition described herein, comprises one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas.

In embodiments, the substantially complete sinonasal microbial composition described herein, comprises one or more of Bifidobacterium, Corynebacterium, Staphylococcus, Streptococcus, Dolosigranulum, Moraxella, Haemophilus, Moraxella, Pseudomonas, Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellagioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus or Lentilactobacillus.

In embodiments, the substantially complete sinonasal microbial composition comprises sinonasal microbes capable of colonizing the sinonasal cavities of a recipient subject for a time period of at least 1 month, 2 months, 3 months, 4 months, 5 months, 6 months or longer.

### BRIEF DESCRIPTION OF THE FIGURES

FIG. 1. Administration of substantially complete sinonasal microbial composition ameliorates CRS symptoms.

The SNOT-22 score is a measure to assess the sinonasal symptoms of subjects having chronic rhinosinusitis (CRS). Patients with diagnosed CRS without polyps (CRSsNP) (n=22) received a 2-week antibiotic pre-treatment, after which participants received daily administration of a substantially complete sinonasal microbial composition comprising sinonasal microbiota derived from healthy donors, for five consecutive days. CRS symptoms were determined prior to starting the treatment (“baseline”), and three months after completion of the treatment (“follow-up”). The average SNOT-22 score was significantly reduced by 38% (p=0.018).

FIG. 2. Administration of substantially complete sinonasal microbial composition ameliorates CRS symptoms in over 70% of CRS subjects.

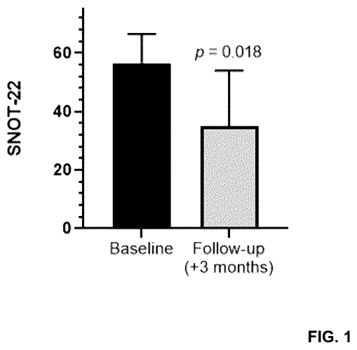

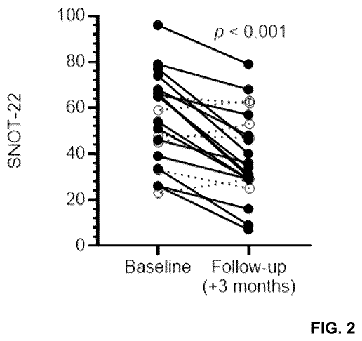

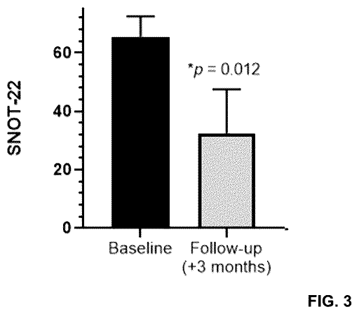

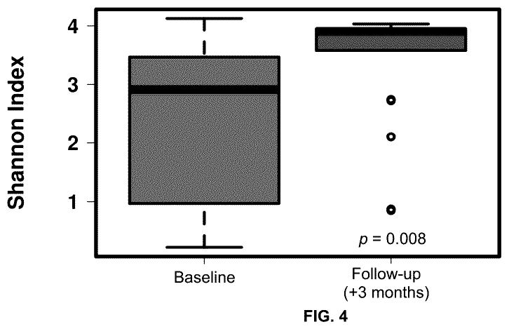

Individual SNOT-22 scores of all subjects with diagnosed CRS without polyps (n=22, see FIG. 1) are shown. 16 out of 22 participants (72.7%) demonstrated a clinically significant reduction (≥9 points) on the SNOT-22 symptom scale compared to baseline. Each of these responders is indicated by a solid circle, wherein the two scores of any subject are connected by a solid line. Non-responders (6 out of 22) show no or only mild improvements and are shown as open circles with dotted lines. A significant reduction in SNOT-22 score was observed across all participants (p<0.001).

FIG. 3. Responders show a clinically relevant reduction of CRS symptoms after administration of substantially complete sinonasal microbial composition.

The responders shown in FIG. 2 (solid circles) exhibited a clinically relevant reduction of the severity and extent of CRS symptoms, shown by a 54.5% reduction in the symptom-based SNOT-22 score (p=0.012).

FIG. 4. Microbial diversity is increased after administration of substantially complete sinonasal microbial composition.

The Shannon Index is a measure to assess the microbial diversity. Patients with diagnosed CRS without polyps (CRSsNP) (n=22) show a wide dispersion of their microbial diversity. After receiving treatment with a substantially complete sinonasal microbial composition comprising sinonasal microbiota derived from healthy donors, a significant increase in microbial diversity was observed. Increased microbial diversity in the sinonasal passages was previously demonstrated in healthy people with no history of sinus disease compared to patients with CRS. The increased diversity was consistent with the improvement in SNOT-22 score.

### DETAILED DESCRIPTION

Provided herein are methods to treat upper respiratory tract inflammatory diseases and conditions. In embodiments, the upper respiratory tract inflammatory diseases and conditions include (chronic) sinusitis, (chronic) rhinitis, and/or (chronic) rhinosinusitis (with or without polyps), as well as nasal polyposis. Further provided herein are substantially complete sinonasal microbial compositions useful for the treatment of upper respiratory tract inflammatory diseases and conditions. Further provided herein are methods of producing substantially complete sinonasal microbial compositions. Further provided herein are medical kits comprising a substantially complete sinonasal microbial composition. The medical kits are useful for delivery and administration of the substantially complete sinonasal microbial compositions to subjects in need thereof, such as, e.g., subjects exhibiting one or more symptoms of an upper respiratory tract inflammatory diseases and conditions, such as, e.g., (chronic) sinusitis, (chronic) rhinitis, and/or (chronic) rhinosinusitis (with or without polyps), as well as nasal polyposis.

In embodiments, administration of the substantially complete sinonasal microbial compositions to subjects in need thereof provides an efficacious treatment to reduce the symptoms of upper respiratory tract inflammatory diseases and conditions, particularly chronic forms of sinusitis, rhinitis, and rhinosinusitis and improve the quality of life of those suffering from chronic forms of these diseases and conditions.

In embodiments, administration of the substantially complete sinonasal microbial compositions to subjects in need thereof provides a therapeutic approach that can, if desired, be combined with standard-of-care therapies, including use of antimicrobials and steroids but which reduces the need (e.g., reduce frequency, reduced dose) of such therapies and thus lowers the burden of side-effects associated with standard-of-care therapies and long-term complications of chronic use (e.g., of antibiotics and steroids) for patients. For example, long-term use of antibiotics may promote the expansion of resistant bacteria, while long-term use of steroids may negatively affect organs, bones, body fat, body weight, increase risk of infections etc.

In embodiments, the therapeutic approach described herein reduces the rate at which the disease or condition, e.g., chronic sinusitis, chronic rhinitis, and/or chronic rhinosinusitis becomes refractory or recalcitrant. In embodiments, the therapeutic approach described herein reduces the need for surgery. For example, antibiotics and steroids may provide good short-term efficacy, however, the effects are diminished over longer periods of time and symptoms persist or worsen.

In embodiments, administration of the substantially complete sinonasal microbial compositions to subjects in need thereof restores a healthy host-microbial balance in the upper respiratory tract. In embodiments, administration of the substantially complete sinonasal microbial compositions to subjects in need thereof restores a healthy host-microbial balance in the sinonasal cavity.

### Methods of Treatment

Provided herein are methods to treat upper respiratory tract inflammatory diseases and conditions in human subjects including (chronic) sinusitis, (chronic) rhinitis, and/or (chronic) rhinosinusitis (with or without polyps), as well as nasal polyposis. The methods include methods of reducing the number of repeated sinus medical therapy and/or sinus surgery in a subject in need of such treatment. These methods include (a) optionally, cleaning the sinonasal cavities, and (b) administering a substantially complete sinonasal microbial composition to the sinonasal cavities. The treatments can be administered (a) prior to sinus surgery (e.g., to raise the threshold for or postpone the need of sinus surgery, or to avoid surgery altogether), (b) after (or together with) sinus surgery (e.g., to prevent recurrence of the disease, including recurrence of polyps), or to treat refractory or recalcitrant chronic forms of the diseases (chronic sinusitis, chronic rhinitis, and chronic rhinosinusitis (with or without polyps)) that are not sufficiently addressed by standard of care treatment (e.g., antimicrobials, corticosteroids and decongestants) and/or sinus surgery. In embodiments, the methods provided herein reduce the number of repeated sinus medical therapy and/or sinus surgery, e.g., to medically address the condition/disease. In some embodiments, the methods provided herein reduce the rate (e.g., number of incidences) of recurrence of the condition/disease, e.g., one or more symptoms. In some embodiments, the methods provided herein are combined with one or more standard of care treatments and reduce, e.g., the number of standard of care treatments necessary to address the condition/disease, the frequency of standard of care treatments necessary to address the condition/disease, and/or the doses (e.g., amount of active agent) required for standard of care treatments to address the condition/disease. In embodiments, reduction in number and/or doses of standard of care treatments (e.g., antibiotics, steroids) reduces the severity and/or number of side-effects exhibited by a subject using the standard of care treatments, including, in embodiments, a reduction in the number and/or severity of long-term deleterious health effects of chronic use of the standard of care treatments.

In embodiments, the methods provided herein treat sinusitis (including chronic sinusitis) in a subject in need of such treatment. In embodiments, the methods provided herein treat rhinitis (including chronic rhinitis) in a subject in need of such treatment. In embodiments, the methods provided herein treat rhinosinusitis (including chronic rhinosinusitis) in a subject in need of such treatment.

The methods described herein include optional cleaning of the sinonasal cavities, e.g., to (at least partially) remove debris/solids, mucus and microbes. In embodiments, cleaning includes at least partial removal of a biofilm comprising sinonasal microbes or other debris from at least a portion of the sinonasal cavities. Cleaning can include one or more of: sinonasal scrubbing, endoscopy (including endoscopic surgery, endoscopic removal), antimicrobial photodynamic therapy, antimicrobial agent treatment, surfactant treatment, mucolytic treatment, salt washes (e.g., iodine)/salt rinses or a combination thereof. In embodiments, cleaning is performed by a physician or clinician (e.g., endoscopy (including endoscopic surgery), antimicrobial photodynamic therapy). In embodiments, cleaning is performed by the subject (e.g., rinsing). Cleaning of the mucosal lining of the sinuses may be performed to achieve one or more of the following: a) disrupting the structure of present biofilm, b) decreasing the viability of microbial populations of the sinonasal passages, c) providing space, e.g., on the mucosal lining for administering the substantially complete sinonasal microbial composition in the sinonasal cavities and/or engraftment of one or more microbial taxa (e.g., bacterial strains comprised in the substantially complete sinonasal microbial composition) and subsequent population doubling, d) preventing the formation of new biofilm and/or facilitating the subject's physiology including the immune system to prevent the formation of new biofilm. In embodiments, the methods described herein may be used to modulate the microbiome in the sinonasal cavities of a subject. In embodiments, the changes in the microbial composition can be detected, e.g., by methods described herein, e.g., after a predetermined period of time, e.g., to determine transient or more durable colonization, e.g., of the sinonasal cavities of a subject, e.g., with one or more microbial constituents of the substantially complete sinonasal microbial composition described herein that has been administered to the subject.

The method described herein include administration of the substantially complete sinonasal microbial composition described herein to the sinonasal cavities. An effective dose or effective amount of the substantially complete sinonasal microbial composition described herein is administered, e.g., effective to treat (chronic) sinusitis, (chronic) rhinitis, and/or (chronic) rhinosinusitis (with or without polyps), and/or effective to reduce the number of repeated sinus medical therapy and/or sinus surgery in a subject in need of such treatment, and/or effective to treat nasal polyposis (e.g., reduce the number of (nasal) polyps) and/or effective to reduce inflammation (e.g., associated with or caused by pathogens in the sinonasal cavities), e.g., of the sinonasal cavities or upper airway tract, and/or effective to reduce unwanted microbial growth (e.g., of the sinonasal cavities with a pathogen).

The substantially complete sinonasal microbial composition described herein used in the methods described herein comprises sinonasal microbes derived from the sinonasal cavities of a healthy subject (e.g., a sinonasal sample donor). The sinonasal microbes can be derived, e.g., (a) from the sinus microbiota (e.g., the microbial community preferentially residing in the sinus cavities), (b) from the nasal microbiota (e.g., the microbial community preferentially residing in the nasal cavities), or (c) derived from both the sinus microbiota and the nasal microbiota (e.g., sinonasal microbiota). In embodiments, the substantially complete sinonasal microbial composition comprises nasal saline wash, nasal mucus, or nasal discharge, e.g., derived from the sinonasal cavities of a healthy subject (e.g., a sinonasal sample donor). In embodiments, the substantially complete sinonasal microbial composition comprises mucus from the sinonasal cavities, e.g., derived from the sinonasal cavities of a healthy subject (e.g., a sinonasal sample donor).

The recipient subject (in need of treatment) or patient receiving the substantially complete sinonasal microbial composition described herein and benefiting from treatment, in some embodiments, exhibits one or more symptoms of chronic sinusitis, chronic rhinitis, chronic rhinosinusitis, nasal polyposis or refractory or recalcitrant versions thereof, e.g., refractory or recalcitrant chronic sinusitis, refractory or recalcitrant chronic rhinitis, refractory or recalcitrant chronic rhinosinusitis, refractory or recalcitrant nasal polyposis. In some embodiments, the standard medical or surgical therapy is not effective (or is effective but needs to be administered chronically, e.g., over many years or the lifetime of the patient), involves too much risk or is otherwise contraindicated, e.g., development of unwanted side-effects, e.g., due to chronic use of standard-of-care drugs. In embodiments, the methods described herein may be used as complementary treatment to endoscopic sinus surgery. In embodiments, the methods described herein may be used as a substitute for endoscopic sinus surgery. In embodiments, the methods described herein may be used to modulate the microbiome in the sinonasal cavities.

The recipient subject (in need of treatment) or patient receiving the substantially complete sinonasal microbial composition described herein and benefiting from treatment, in some embodiments has been diagnosed with, is suspected of having, or is at (increased) risk of developing a sinusitis, including, e.g., acute sinusitis, sub-acute sinusitis, chronic sinusitis, allergic sinusitis, or non-allergic sinusitis. In embodiments, increased risk is assessed relative to a control.

The recipient subject (in need of treatment) or patient receiving the substantially complete sinonasal microbial composition described herein and benefiting from treatment, in some embodiments has been diagnosed with, is suspected of having, or is at (increased) risk of developing a rhinitis, including, e.g., allergic rhinitis and non-allergic rhinitis.

The recipient subject (in need of treatment) or patient receiving the substantially complete sinonasal microbial composition described herein and benefiting from treatment, in some embodiments has been diagnosed with, is suspected of having, or is at (increased) risk of developing a rhinosinusitis, including, e.g., allergic rhinosinusitis and non-allergic rhinosinusitis.

The recipient subject (in need of treatment) or patient receiving the substantially complete sinonasal microbial composition described herein and benefiting from treatment, in some embodiments has been diagnosed with, is suspected of having, or is at (increased) risk of developing a chronic sinusitis (CS) or chronic rhinosinusitis (CRS).

The recipient subject (in need of treatment) or patient receiving the substantially complete sinonasal microbial composition described herein and benefiting from treatment, in some embodiments has been diagnosed with, is suspected of having, or is at (increased) risk of developing a refractory or recalcitrant rhinosinusitis. A refractory or recalcitrant form of the condition/disease is characterized by not being sufficiently addressed by standard of care treatment (e.g., antimicrobials, corticosteroids and decongestants) and/or sinus surgery, e.g., it exhibits one or more of: high resistance to treatment, poor therapeutic response, high recurrence rate of the disease.

The recipient subject (in need of treatment) or patient, specifically a chronic patient (e.g., chronic rhinosinusitis), receiving the substantially complete sinonasal microbial composition described herein and benefiting from treatment, in some embodiments is at increased risk of developing nasal polyposis than a control. CRS is clinically classified into CRS with nasal polyps and CRS without nasal polyps. CRS with nasal polyps can be divided into eosinophilic and non-eosinophilic according to the degree of eosinophil infiltration. The degree of eosinophil infiltration in nasal polyp tissue is most closely related to recurrence. Patients with an eosinophilic phenotype tend to have a poor response to medical and surgical management.

In embodiments, methods are provided herein for treating chronic sinusitis with nasal polyps/nasal polyposis. In embodiments, methods are provided herein for treating chronic sinusitis without nasal polyps/nasal polyposis. In embodiments, methods are provided herein for treating chronic sinusitis with eosinophilic phenotype. In embodiments, methods are provided herein for treating chronic sinusitis with non-eosinophilic phenotype.

The recipient subject (in need of treatment) or patient, specifically an elderly patient, receiving the substantially complete sinonasal microbial composition described herein and benefiting from treatment, in some embodiments has been diagnosed with, is suspected of having, or is at (increased) risk of developing a bronchitis and pneumonia, chronic or acute viral infections of the respiratory tract, or chronic or acute bacterial infections of the respiratory tract.

The recipient subject (in need of treatment) or patient receiving the substantially complete sinonasal microbial composition described herein and benefiting from treatment, in some embodiments has been diagnosed with, is suspected of having, or is at (increased) risk of developing a comorbidity, e.g., asthma, cystic fibrosis, ciliary dyskinesia or other inflammatory condition. In some embodiments, the recipient subject (in need of treatment) or patient receiving the substantially complete sinonasal microbial composition described herein is an active cigarette smoker or has been a cigarette smoker in the past 10 years.

The recipient subject (in need of treatment) or patient receiving the substantially complete sinonasal microbial composition described herein and benefiting from treatment, in some embodiments exhibits one or more of: asthma, methicillin resistant Staphylococcus aureus (MRSA) infection, Samter's triad/polyposis (Aspirin Exacerbated Respiratory Disease (AERD)), and/or sinonasal sarcoidosis.

The recipient subject (in need of treatment) or patient receiving the substantially complete sinonasal microbial composition described herein and benefiting from treatment, in some embodiments exhibits gastroesophageal reflux disease (GERD) or an immunodeficiency.

The recipient subject (in need of treatment) or patient receiving the substantially complete sinonasal microbial composition described herein and benefiting from treatment, in some embodiments exhibits upper airway inflammation. The recipient subject (in need of treatment) or patient receiving the substantially complete sinonasal microbial composition described herein and benefiting from treatment, in some embodiments exhibits inflammation of the sinonasal cavities. In embodiments, the inflammation is associated with Th2-mediated inflammatory pathways. Associated Th2 inflammatory biomarkers include, e.g., one or more of: IL-4, IL-5, IL-13, ECP, and/or P-gp. In embodiments, the inflammation is associated with Th1/Th17-mediated inflammatory pathways. Associated Th1/Th17 inflammatory biomarkers include, e.g., one or more of: IFN-gamma, TNF-alpha, and/or IL-17A.

The recipient subject (in need of treatment) or patient receiving the substantially complete sinonasal microbial composition described herein and benefiting from treatment, in some embodiments exhibits upper airway inflammation, including inflammation of the sinonasal cavities, caused by an allergen or irritant.

The recipient subject (in need of treatment) or patient receiving the substantially complete sinonasal microbial composition described herein and benefiting from treatment, in some embodiments exhibits upper airway inflammation of the sinonasal cavities, caused by a (respiratory) pathogen, e.g., a virus, fungus or bacterium.

The recipient subject (in need of treatment) or patient receiving the substantially complete sinonasal microbial composition described herein and benefiting from treatment, in some embodiments exhibits a pro-inflammatory resident microbiome (microbial community), e.g., in the nasal and or sinus cavities (niche), e.g., microbial communities comprising taxa (e.g., bacterial taxa) that elicit a Th1 and/or Th2 host immune response, including a chronic immune response.

The recipient subject (in need of treatment) or patient receiving the substantially complete sinonasal microbial composition described herein and benefiting from treatment, in some embodiments exhibits (chronic) sinusitis, (chronic) rhinitis, and/or (chronic) rhinosinusitis (e.g., allergic and non-allergic sinusitis; allergic and non-allergic rhinitis; allergic an non-allergic rhinosinusitis) and one or more of the following (concomitant) conditions: a (chronic) infection (e.g., bacterial (e.g., Staphylococcus aureus) infection or fungal infection), asthma (e.g., moderate-to-severe asthma) and/or another Th2-inflammatory systemic condition, aspirin hypersensitivity, non-steroidal anti-inflammatory drug (NSAID) hypersensitivity, and Samter's triad (defined by presence of nasal polyps, asthma, and aspirin and NSAID sensitivity), optionally wherein the subject has one or more (two or more, three or more) nasal polyps (e.g., in each nostril), including bilateral nasal polyposis, and/or may have undergone surgery for nasal polyps. In other embodiments, the subject has cystic fibrosis or NARES (non-allergic rhinitis with eosinophilia syndrome).

In one embodiment, the recipient subject (in need of treatment) or patient receiving the substantially complete sinonasal microbial composition described herein and benefiting from treatment, exhibits cystic fibrosis (CF). CRS is present in most CF patients and often refractory to standard therapy. In addition, the rate of recurrences after surgical treatment are very high. In some embodiment, the recipient subject (in need of treatment) or patient receiving the substantially complete sinonasal microbial composition described herein is a CF patient with (refractory) CRS.

A subject or patient in need of treatment, e.g., has, is suspected of having or is at risk of developing (chronic) sinusitis, (chronic) rhinitis, and (chronic) rhinosinusitis (with or without polyps, e.g., nasal polyposis) and/or various subtypes thereof (e.g., allergic or non-allergic rhinitis, or allergic or non-allergic rhinosinusitis) and can be diagnosed, e.g., by detecting mucin levels, mucosal inflammation, or diagnosing one or more (e.g., two, three, or four, preferably, at least two) symptoms, such as, e.g., nasal congestion/obstruction, facial discomfort (pain/pressure, tenderness and swelling around the patient's eyes, cheeks, nose or forehead), nasal discharge, headache, hyposmia/anosmia, as well as other symptoms such as, e.g., ear pain/pressure, aching in the upper jaw and teeth, cough, sore throat, fatigue, irritability, and nausea. In embodiments, the subject presents with at least two of the following symptoms: nasal blockade/obstruction/congestion or nasal discharge (anterior/posterior nasal drip); facial pain/pressure; and reduction or loss of smell.

Anatomical characterization, e.g., of the sinonasal mucosal lining, e.g., to diagnose or confirm a diagnosis (e.g., of a subject or patient in need of treatment) can be accomplished using standard imaging techniques. Nasal polyposis can be diagnosed, e.g., on the basis of sinonasal computed tomography (CT) scan and/or sinonasal endoscopy. Microbes residing in the sinonasal microbiome can be detected using nucleic acid techniques, e.g., arrays, hybridization, or PCR, using sequences complementary to species- or order-specific nucleic acid sequences.

A subject or patient in need of treatment, e.g., has, is suspected of having, or is at risk of developing (chronic) sinusitis, (chronic) rhinitis, and (chronic) rhinosinusitis (with or without polyps, e.g., nasal polyposis) and/or various subtypes thereof (e.g., allergic or non-allergic rhinitis, or allergic or non-allergic rhinosinusitis) and can be diagnosed, e.g., by one or more of: (a) 20 or 22-item Sino Nasal Outcome Test (SNOT-20, SNOT-22) score; (b) subject-assessed nasal congestion/obstruction, anterior rhinorrhea, posterior rhinorrhea and loss of sense of smell; (c) number of nightly awakenings; (d) Visual Analog Score (VAS) to assess patient-rated rhinosinusitis symptom severity; (e) five-item Asthma Control Questionnaire (ACQ5) score in patients with asthma; (f) Nasal Polyp Score (NPS); (g) Nasal Peak Inspiratory Flow (NPIF); (h) smell test (University of Pennsylvania Smell Identification Test (UPSIT); (i) physiological parameters, such as measured by nasal endoscopy and CT scan; (j) Lund-Mackay Score; and (k) Three Dimensional volumetric measurement of the maxillary sinus.

The methods to treat upper respiratory tract inflammatory diseases and conditions in human subjects including (chronic) sinusitis, (chronic) rhinitis, and/or (chronic) rhinosinusitis (with or without polyps), as well as nasal polyposis described herein comprising administering the substantially complete sinonasal microbial composition described herein can be combined with any standard of care treatment regimen as determined by a treating physician or clinician, or may be provided as a substitute for a standard of care treatment regimen. Concurrent administration of two or more therapeutic agents (including the substantially complete sinonasal microbial composition described herein) does not require that the agents be administered at the same time or by the same route, provided there is overlap in the time period during which the agents are exerting their therapeutic effect. Simultaneous or sequential administration is contemplated, as is administration on different days or weeks. The substantially complete sinonasal microbial composition described herein (in an effective dose) may be administered to a subject in need thereof either prior to, concurrent with, or after a standard of care treatment regimen, e.g., prior to, concurrent with, or after treatment with one or more of antimicrobials, corticosteroids and/or decongestants, or other active agents, such as, e.g., anti-inflammatory agents/corticosteroids, anti-septic (e.g., iodine), mucolytic, antihistamine medication, decongestant, vasoconstrictor, or a chelating agent medication.

Current treatment of (chronic) sinusitis, (chronic) rhinitis, and/or (chronic) rhinosinusitis (with or without polyps) is primarily aimed at reducing sinonasal inflammation, keeping nasal passages draining, eliminating the underlying cause (e.g., pathogen or allergen), and/or reducing the number of sinusitis flare-ups. The most common treatments for chronic sinusitis include steam or mist inhalation, antimicrobials, corticosteroids, and decongestants which are often successful in reducing discomfort (mucosal swelling) and relieving obstruction of the sinus ostium (drainage). For example, one treatment regimen involves saline nasal irrigation, typically with nasal sprays or solutions, to increase drainage and rinse away irritants and allergens. Other treatment regimens include nasal corticosteroids, typically in the form of nasal sprays to help prevent and treat inflammation (e.g., in patients with signs and symptoms suggestive of allergic sinusitis, and patients with an exacerbation of chronic sinusitis). Examples of anti-inflammatory corticosteroid nasal sprays include fluticasone, triamcinolone, budesonide, mometasone, flunisolide and beclomethasone. If sprays are insufficient, rinsing with a solution of saline and budesonide or using a nasal mist is often recommended. Oral or injected corticosteroids (e.g., prednisone and methylprednisolone) are also employed, typically to relieve inflammation from severe sinusitis. Prolonged use of topical steroids and short bursts of oral steroid therapy may help to reduce swelling and relieve osteomeatal obstruction. Decongestants, e.g., pseudoephedrine or phenylpropanolamine and over-the-counter pain relievers, e.g., aspirin, acetaminophen or ibuprofen, may also be administered. In embodiments, treatment comprises an intranasal corticosteroid optionally in combination with a long-acting beta2-adronergic antagonist (LABA), e.g., for a subject exhibiting asthma. Other treatment regimens may include one or more of: nasal saline, a topical decongestant, a topical anesthetic, a leukotriene antagonist or an antihistamine. Topical medications include antihistamine, anticholinergic, antibiotic, and antifungal sprays, among others. Allergic patients also benefit from the use of antihistamines and/or immunotherapy or desensitization. Aspirin desensitization treatment is also employed if reaction to aspirin is the cause of the sinusitis. Any such treatments may be combined with methods comprising administering the substantially complete sinonasal microbial composition described herein, e.g., as directed by a physician or clinician.

Further treatment options include administration of antimicrobials and immune-/anti-inflammatory therapy, as well as surgery (including (functional) endoscopic sinus surgery (FESS/ESS) and balloon sinuplasty), e.g., to remove tissue that may be causing nasal blockage (including nasal polyps) and/or enlarge narrow sinus openings. Surgery is often performed, e.g., to remove the obstruction in the anterior ethmoids. Any such treatments may be combined with methods comprising administering the substantially complete sinonasal microbial composition described herein, e.g., as directed by a physician or clinician. For example, the substantially complete sinonasal microbial composition described herein may be administered concurrent with or after sinus surgery.

A broad-spectrum antibiotic can be chosen if desired, e.g., to treat (underlying) unwanted microbial (over)growth of sinonasal pathogens (e.g., H. influenzae, S. pneumoniae, and B. catarrhalis). Amoxicillin and other penicillins, as well as cephalosporins (e.g., cefpodoxime, ceftibuten, cefuroxime, loracarbef, and cefaclor), may be used as an initial treatment. Alternatives include, e.g., amoxicillin/clavulanate potassium, e.g., for chronic sinusitis. Clarithromycin may be given to patients who are allergic to penicillins. Acute sinusitis frequently manifests with heavy microbial growth of a predominant pathogen, but chronic sinusitis is typically associated with polymicrobial growth in which anaerobes are often present, particularly suitable for broad-spectrum antibiotic. In embodiments, the antibiotic compound is a beta-lactam, a cephalosporin, a lincosamide, a macrolide, a tetracycline, a sulfa drug (e.g., compound), or mupirocin. In embodiments, the antibiotic compound is oxacillin, flucloxacillin, cefazolin, cephalothin, cephalexin, erythromycin, doxycycline, or minocycline. In embodiments, the antibiotic compound is erythromycin, penicillin G, clarithromycin, bactrim DS, ciprofloxacin, vancomycin, daptomycin, or linezolid. In embodiments, the antibiotic compound is a penicillin, a cephalosporin, a monobactam, a fluoroquinolone, a carbapenem, an aminoglycoside, or a polymixin. Examples of beta-lactam antibiotics include: penicillin derivatives (penams), cephalosporins (cephems), monobactams, and carbapenems, penicillin G, penicillin V, methicillin; oxacillin, nafcillin, ampicillin, amoxicillin, and carbenicillin. Examples of lincosamides include: lincomycin, clindamycin, and pirlimycin. Examples of macrolides include: azithromycin, clarithromycin, erythromycin, fidaxomicin, and telithromycin. Examples of tetracyclines include: tetracycline, chlortetracycline, oxytetracycline, demeclocycline, lymecycline, meclocycline, methacycline, minocycline, and rolitetracycline. Examples of sulfa drugs include: co-trimoxazole, sulfadiazine, sulfamethoxazole, trimethoprim-sulfamethoxazole, trimethoprim, sulfasalazine, and sulfisoxazole. Examples of cephalosporins include: ceftobiprole, ceftaroline, ceftolozane, cefclidine, cefepime, cefluprenam, cefoselis, cefozopran, cefpirome, and cefquinome. Examples of monobactams include: aztreonam, tigemonam, nocardicin A, and tabtoxin. Examples of fluoroquinolones include: ciprofloxacin, garenoxacin, gatifloxacin, gemifloxacin, levofloxacin, and moxifloxacin. Examples of carbapenems include: imipenem, meropenem, ertapenem, and doripenem. Examples of aminoglycosides include: kanamycin A, amikacin, tobramycin, dibekacin, gentamicin, sisomicin, netilmicin, neomycins B, C, paromomycin, and streptomycin. Examples of polymixins include: mattacin, polymyxin B, and colistin. Therapeutic regimens include the combination of a penicillin such as amoxicillin plus a beta-lactamase inhibitor, a combination of metronidazole plus a macrolide or a second or third generation cephalosporin, and the new quinolones. Where aerobic gram-negative organisms are suspected, therapy with an aminoglycoside, fourth generation cephalosporin or fluoroquinolone may be added. Antibiotic coverage for MRSA is often also included. Any such treatments may be combined with methods comprising administering the substantially complete sinonasal microbial composition described herein, e.g., as directed by a physician or clinician.

In embodiments, prolonged antimicrobial therapy (up to several weeks) may be required.

The methods comprising administering the substantially complete sinonasal microbial composition described herein, e.g., to treat (chronic) sinusitis, (chronic) rhinitis, and/or (chronic) rhinosinusitis (with or without polyps), may comprise one or more additional therapies described herein, including a combination of approaches that may include one or more of topical or oral glucocorticoids/steroids, antimicrobials, decongestants, nasal saline irrigations, and surgery, as directed by a physician or clinician. Treatment goals may vary and can change over the course of treatment and may include one or more of: reduction of mucosal edema, reduction of inflammation, promotion of sinus drainage, and/or elimination or reduction of pathogens or allergens. Combination therapies contemplate coadministration, using separate formulations or a single pharmaceutical formulation, and consecutive administration in any order.

In embodiments, the methods comprising administering the substantially complete sinonasal microbial composition described herein, may be used to reduce the need (e.g., reduce the frequency of dosing and/or reduce the dose (e.g., reduce the amount of active agent) for one or more additional therapies described herein, e.g., to reduce symptoms or maintain a symptom-free state. For example, the need (e.g., frequency/dose) for one or more of glucocorticoids/steroids, antimicrobials, and/or decongestants is reduced. In embodiments, the reduced need also reduces the side-effects of the one or more additional therapies described herein, thereby benefiting the patient.

In embodiments, methods comprising administering the substantially complete sinonasal microbial composition described herein, e.g., to treat (chronic) sinusitis, (chronic) rhinitis, and/or (chronic) rhinosinusitis (with or without polyps), may comprise controlling microbial growth, e.g., at the mucosal level. In embodiments, methods comprising administering the substantially complete sinonasal microbial composition described herein, e.g., to treat (chronic) sinusitis, (chronic) rhinitis, and/or (chronic) rhinosinusitis (with or without polyps), may comprise controlling inflammation. In embodiments, methods comprising administering the substantially complete sinonasal microbial composition described herein, e.g., to treat (chronic) sinusitis, (chronic) rhinitis, and/or (chronic) rhinosinusitis (with or without polyps), may comprise controlling microbial growth and inflammation. For example, the method described herein include optional cleaning of the sinonasal cavities comprising at least partial removal of a biofilm comprising sinonasal microbes or other debris from at least a portion of the sinonasal cavities. In some embodiments, cleaning the sinonasal cavities comprises at least a 1-log reduction of the sinonasal microbes comprised in the biofilm of at least a portion of the sinonasal cavities of the subject. In some embodiments, cleaning the sinonasal cavities comprises at least a 2-log reduction of the sinonasal microbes comprised in the biofilm of at least a portion of the sinonasal cavities of the subject.

In embodiments, microbial biofilms cause and/or perpetuate inflammation, e.g., associated with refractory or recalcitrant sinusitis, rhinitis or rhinosinusitis, and inflammation damages the epithelium, impairs local immune defenses and in turn favors microbial (e.g., bacterial) attachment with biofilms formation. The inflammatory process may include increased capillary permeability, leukocytes infiltration, angiogenesis and edema. In embodiments, corticosteroids may be administered, e.g., to block formation of inflammatory mediators, inhibit chemotaxis of inflammatory cells, inhibit angiogenesis, and/or inhibit allergic hypersensitivity reaction, and may optionally be combined with one or more antimicrobials.

Administrations of a standard of care agent and/or the substantially complete sinonasal microbial composition described herein can be single or multiple times (e.g., 2- or 3-, 4-, 6-, 8-, 10-, or more times), e.g., daily, weekly, monthly, or yearly. The duration of treatments (e.g., a treatment regimen) provided herein may range from one day to week(s), month(s), year(s) or the lifetime of a subject.

Treatment progression in a subject, e.g., treated using a method described herein, e.g., comprising administering the substantially complete sinonasal microbial composition described herein may be measured, e.g., by monitoring or assessing the severity and/or frequency of one or more symptoms associated with (chronic) sinusitis, (chronic) rhinitis, and/or (chronic) rhinosinusitis (with or without polyps), e.g., nasal congestion/obstruction, facial discomfort (pain/pressure, tenderness and swelling around the patient's eyes, cheeks, nose or forehead), nasal discharge, headache, hyposmia/anosmia, as well as other symptoms such as, e.g., ear pain/pressure, aching in the upper jaw and teeth, cough, sore throat, fatigue, irritability, nausea, polyp formation, and rate of recurrence thereof. In embodiments, the method for treatment described herein, e.g., comprising administering the substantially complete sinonasal microbial composition described herein, reduce the severity and/or frequency of one or more symptoms associated with (chronic) sinusitis, (chronic) rhinitis, and/or (chronic) rhinosinusitis (with or without polyps). In embodiments, the reduction is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, reduce the rate of recurrence of one or more symptoms associated with (chronic) sinusitis, (chronic) rhinitis, and/or (chronic) rhinosinusitis (with or without polyps). In embodiments, the method for treatment described herein, e.g., comprising administering the substantially complete sinonasal microbial composition described herein, prevent the recurrence of one or more symptoms associated with (chronic) sinusitis, (chronic) rhinitis, and/or (chronic) rhinosinusitis (with or without polyps), e.g., for the period of 1 week, 2 weeks, 3 weeks, 1 month, 2 months, 4 months, 6 months, 8 months, 10 months, 1 year, 2 years, 3 years, 5 years, 10 years, or more than 10 years.

Treatment progression in a subject, e.g., treated using a method described herein, e.g., comprising administering the substantially complete sinonasal microbial composition described herein may be measured, e.g., by monitoring or assessing one or more inflammatory biomarkers, e.g., locally, e.g., in the sinonasal cavities, or systemically, e.g., by obtaining and analyzing a biological sample, e.g., to determine the presence or absence of one or more pro- or anti-inflammatory markers, e.g., interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), interferons (e.g., IFN-7), and/or Periostin, eosinophil cationic protein (ECP), P-glycoprotein (P-gp). In some embodiment, treatment progression is determined by determining the level of IL-5, IL-13, IL-2, IL-6, IL-10, IL-4, and/or IFN-γ.

In embodiments, treatment progression is determined by Lund-Mackay (LM) CT score, SNOT-22 score and/or the endoscopic Modified Lund-Kennedy (MLK) score.

In embodiments, the method for treatment described herein, e.g., comprising administering the substantially complete sinonasal microbial composition described herein, reduce the presence of one or more pro-inflammatory markers, e.g., in the sinonasal cavities and/or upper respiratory tract. In embodiments, the reduction is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, reduce the rate of recurrence of inflammation and/or colonization (e.g., by pathogens), e.g., in the sinonasal cavities and/or upper respiratory tract. In embodiments, the method for treatment described herein, e.g., comprising administering the substantially complete sinonasal microbial composition described herein, prevent the recurrence of inflammation, e.g., in the sinonasal cavities and/or upper respiratory tract, e.g., for the period of 1 week, 2 weeks, 3 weeks, 1 month, 2 months, 4 months, 6 months, 8 months, 10 months, 1 year, 2 years, 3 years, 5 years, 10 years, or more than 10 years.

Treatment progression in a subject, e.g., treated using a method described herein, e.g., comprising administering the substantially complete sinonasal microbial composition described herein may be measured, e.g., by monitoring or assessing the microbiome constituents, e.g., of the nasal and/or sinus microbiome residing in the nasal and/or sinus cavities (niche), e.g., by obtaining and analyzing a biological sample, e.g., by sequencing to determine the presence or absence of one or more pathogens. In embodiments, the method for treatment described herein, e.g., comprising administering the substantially complete sinonasal microbial composition described herein, reduce the presence of one or more pathogens, e.g., in the sinonasal cavities and/or upper respiratory tract. In embodiments, the reduction is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, reduce the rate of recurrence of unwanted microbial growth, e.g., in the sinonasal cavities and/or upper respiratory tract. In embodiments, the method for treatment described herein, e.g., comprising administering the substantially complete sinonasal microbial composition described herein, prevent the recurrence of unwanted microbial growth, e.g., in the sinonasal cavities and/or upper respiratory tract, e.g., for the period of 1 week, 2 weeks, 3 weeks, 1 month, 2 months, 4 months, 6 months, 8 months, 10 months, 1 year, 2 years, 3 years, 5 years, 10 years, or more than 10 years. In embodiments, the method for treatment described herein, e.g., comprising administering the substantially complete sinonasal microbial composition described herein, reduce the coverage and/or thickness of a biofilm, e.g., on at least a part of the sinonasal cavities (mucosal) lining. In embodiments, the reduction is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, reduce the rate of recurrence of a biofilm, e.g., on at least a part of the sinonasal cavities (mucosal) lining. In embodiments, the method for treatment described herein, e.g., comprising administering the substantially complete sinonasal microbial composition described herein, prevent the recurrence of a biofilm, e.g., on at least a part of the sinonasal cavities (mucosal) lining, e.g., for the period of 1 week, 2 weeks, 3 weeks, 1 month, 2 months, 4 months, 6 months, 8 months, 10 months, 1 year, 2 years, 3 years, 5 years, 10 years, or more than 10 years.

Treatment progression in a subject, e.g., treated using a method described herein, e.g., comprising administering the substantially complete sinonasal microbial composition described herein may be measured, e.g., by monitoring or assessing one or more of: (a) Sino Nasal Outcome Test (e.g., SNOT-20 or SNOT-22) score; (b) nasal congestion/obstruction assessment, comprising one or more of: including anterior rhinorrhea (runny nose), posterior rhinorrhea (post nasal drip) and loss of sense of smell; (c) number of nightly awakenings; (d) visual analog score (VAS); (e) asthma control questionnaire (ACQ5) score; (f) nasal peak inspiratory flow (NPIF); (g) smell test (University of Pennsylvania Smell Identification Test (UPSIT)); (h) assessment of one or more physiological parameters (e.g., by nasal endoscopy or CT scan); (i) Lund-Mackay Score; and (j) volumetric measurements of at least one part of the sinonasal cavities. In embodiments, the method for treatment described herein, e.g., comprising administering the substantially complete sinonasal microbial composition described herein, improve the score or test results of one or more of (a) through (j). In embodiments, the improvement is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, improve the score or test results of one or more of (a) through (j), e.g., for the period of 1 week, 2 weeks, 3 weeks, 1 month, 2 months, 4 months, 6 months, 8 months, 10 months, 1 year, 2 years, 3 years, 5 years, 10 years, or more than 10 years. 22-Item Sinonasal Outcome Test (SNOT-22) Score: In embodiments, the method for treatment described herein, e.g., comprising administering the substantially complete sinonasal microbial composition described herein result in a decrease from baseline of the 22-item Sinonasal Outcome Test (SNOT-22). In embodiments, the method for treatment described herein results in a reduction of the SNOT-22 score by at least 10, 20, 30, 40 or more points.

The SNOT-22 is a questionnaire to assess the impact of chronic rhinosinusitis (CRS) on quality of life. The questionnaire measures items related to sinonasal conditions and surgical treatments. The score ranges from 0 to 110, and higher scores imply greater impact of CRS on Health Related Quality of Life (HRQoL).

In certain aspects, use of the methods provided herein, e.g., administration of the substantially complete sinonasal microbial composition to recipient subject, e.g., a CRS patient without polyps, reduces the recipient subject's SNOT-22 score, e.g., by at least 9 points. In embodiments, a healthy subject, e.g., a healthy donor subject, has a SNOT-22 score of 7 or less. A reduction of the SNOT-22 score of at least 9 points following treatment using the substantially complete sinonasal microbial composition described herein is, in embodiments, considered a clinically meaningful improvement.

Individual and Total Nasal Symptom Score. In embodiments, the method for treatment described herein, e.g., comprising administering the substantially complete sinonasal microbial composition described herein result in a decrease from baseline of the Nasal Symptom Score (e.g., compared to a control). Subject-assessed symptoms are assayed by responding to morning and evening individual rhinosinusitis symptom questions using a 0-3 categorical scale (where 0=no symptoms, 1=mild symptoms, 2=moderate symptoms and 3=severe symptoms) and including the symptoms of congestion and/or obstruction, anterior rhinorrhea, posterior rhinorrhea, and loss of sense of smell. Nasal symptoms can be assayed in the day (AM), at night (PM) or both AM and PM.

In embodiments, the method for treatment described herein, e.g., comprising administering the substantially complete sinonasal microbial composition described herein result in an improvement of the sense of smell (e.g., compared to a control).

In embodiments, the method for treatment described herein, e.g., comprising administering the substantially complete sinonasal microbial composition described herein result in a decrease in congestion and/or obstruction (e.g., compared to a control).

In embodiments, the method for treatment described herein, e.g., comprising administering the substantially complete sinonasal microbial composition described herein result in a decrease in runny nose (e.g., compared to a control).

In embodiments, the method for treatment described herein, e.g., comprising administering the substantially complete sinonasal microbial composition described herein result in a decrease in post-nasal drip (e.g., compared to a control).

In embodiments, the method for treatment described herein, e.g., comprising administering the substantially complete sinonasal microbial composition described herein result in an improvement of night-time sleep (e.g., a decrease in night-time awakenings) (e.g., compared to a control). A measure of night-time awakenings can be assessed according to the following scores based on subject self-assessment: 0=no symptoms, slept through the night; 1=slept well, but some complaints in the morning; 2=woke up once because of rhinosinusitis symptoms (including early awakening); 3=woke up several times because of symptoms (including early awakening); 4=bad night, awake most of the night because of symptoms. In embodiments, the Pittsburgh Sleep Quality Index (PSQI) score is used.

Visual Analog Score (VAS). In embodiments, the method for treatment described herein, e.g., comprising administering the substantially complete sinonasal microbial composition described herein result in an improvement of the VAS. The VAS is a measure to assess patient-related rhinosinusitis symptom severity on a scale of 1 to 10. Mild symptoms are indicated by a score of 0 to 3, moderate symptoms are indicated by a VAS score of >3 to 7, and severe symptoms are indicated by a VAS score of >7 to 10.

University of Pennsylvania Smell Identification Test (UPSIT). In embodiments, the method for treatment described herein, e.g., comprising administering the substantially complete sinonasal microbial composition described herein result in an improvement of the UPSIT. The UPSIT is a method to quantitatively assess human olfactory function. The test consists of samples of odorants, and the subject describes the odor. The score is based on the number of correct answers. This test can distinguish patients with a normal sense of smell (normosmia) from those with different levels of reduction (mild, moderate and severe microsmia/hyposmia) or loss (anosmia).

In embodiments, the method for treatment described herein, e.g., comprising administering the substantially complete sinonasal microbial composition described herein result in an improvement of one or more physiological parameters, such as, e.g., assessment of the nasal cavities, e.g., by nasal endoscopy or computed tomography (CT) scan.

Lund-Mackay Score. In embodiments, the method for treatment described herein, e.g., comprising administering the substantially complete sinonasal microbial composition described herein result in an improvement of the Lund-Mackay score. The Lund-Mackay scoring system is based on localization with points given for degree of opacification: 0=normal, 1=partial opacification, 2=total opacification. These points are then applied to the maxillary, anterior ethmoid, posterior ethmoid, sphenoid, and frontal sinus on each side. The osteomeatal complex is graded as 0=not occluded, or 2=occluded deriving a maximum score of 12 per side.

In embodiments, the method for treatment described herein, e.g., comprising administering the substantially complete sinonasal microbial composition described herein result in an improvement of scores of one or more of quality of life (QoL) questionnaires, such as, e.g., the Short-Form-36 (SF-36) Questionnaire, the Euroqol-5D (EQ-5D), nasal polyp related resource use questionnaire, and patient qualitative self-assessments. For example, the SF-36 is a 36-item questionnaire that measures eight multi-item dimensions of health: physical functioning (10 items) social functioning (2 items) role limitations due to physical problems (4 items), role limitations due to emotional problems (3 items), mental health (5 items), energy/vitality (4 items), pain (2 items), and general health perception (5 items). For each dimension, item scores are coded, summed, and transformed on a scale from 0 (worst possible health state measured by the questionnaire) to 100 (best possible health state). Two standardized summary scores can also be calculated from the SF-36: the physical component summary (PCS) and the mental health component summary (MCS). Another example is the EQ-5D. EQ-5D is a standardized health-related quality of life questionnaire developed by the EuroQol Group in order to provide a simple, generic measure of health for clinical and economic appraisal and inter-disease comparisons. EQ-5D, designed for self-completion by patients, consists of two parts, the EQ-5D descriptive system and the EQ VAS. The EQ-5D descriptive system comprises 5 dimensions: mobility, self-care, usual activities, pain/discomfort and anxiety/depression; and each dimension has 3 levels: no problem, some problems, severe problems. The EQ Visual Analogue Scale (VAS) records the respondent's self-rated health on a vertical visual analogue scale. The EQ VAS ‘thermometer’ has endpoints of 100 (best health state) and 0 (worst health state). The nasal polyp related resource use questionnaire is a questionnaire of health care resource utilization for nasal polyposis, including specialist visits, emergency care visits, sick leaves, days off etc. In some embodiments, the Rhinosinusitis Disability Index (RSDI) is used.

Treatment progression in a subject, e.g., treated using a method described herein, e.g., comprising administering the substantially complete sinonasal microbial composition described herein may be measured, e.g., by monitoring or assessing the endoscopic Nasal Polyp Score. For example, a nasal polyp score of 0 indicates the absence of polyps. An NPS of 1 indicates the presence of small polyps in the middle meatus not reaching below the inferior border of the middle turbinate. In embodiments, the method for treatment described herein, e.g., comprising administering the substantially complete sinonasal microbial composition described herein result in an improvement of the Nasal Polyp Score. In embodiments, the improvement is determined, e.g., relative to the (baseline) level prior to treatment (e.g., in the same subject) or relative to a control (e.g., a control subject or a control population). In embodiments, the method for treatment described herein, e.g., comprising administering the substantially complete sinonasal microbial composition described herein, reduce the rate of recurrence of nasal polyps. In embodiments, the method for treatment described herein, e.g., comprising administering the substantially complete sinonasal microbial composition described herein, prevent the recurrence of polyps (or reduce the number of recurring polyps), e.g., for the period of 1 week, 2 weeks, 3 weeks, 1 month, 2 months, 4 months, 6 months, 8 months, 10 months, 1 year, 2 years, 3 years, 5 years, 10 years, or more than 10 years. In embodiments, the reduction is determined, e.g., relative to the (baseline) level prior to treatment (e.g., in the same subject) or relative to a control (e.g., a control subject or a control population).

In embodiments, a reduction, e.g., a reduction in the number of symptoms, the severity of symptoms, or the frequency of symptoms, or reduction in the coverage area or thickness of a biofilm, is a reduction of at least 5%, 10%, 20%, 30%, 40%, 50%, 60%, 70%, 80%, or at least 90% e.g., relative to the (baseline) level prior to treatment (e.g., in the same subject) or relative to a control (e.g., a control subject or a control population).

In embodiments, a reduction, e.g., a reduction in the level of a pathogen (or microbial load) or an inflammatory biomarker (e.g., a pro-inflammatory biomarker) is a reduction of at least 5%, 10%, 20%, 30%, 40%, 50%, 60%, 70%, 80%, or at least 90%, e.g., relative to the (baseline) level prior to treatment (e.g., in the same subject) or relative to a control (e.g., a control subject or a control population), or a reduction below the limit of detection (substantially absent).

In embodiments, an improvement, e.g., an improvement of a score (e.g., of a test described herein, e.g., Nasal Polyp Score, quality of life (QoL) questionnaire, Lund-Mackay Score, Visual Analog Score, UPSIT, Nasal Symptom Score, Sinonasal Outcome Test (SNOT) score, Pittsburgh Sleep Quality Index (PSQI) score, Rhinosinusitis Disability Index (RSDI), nasal peak inspiratory flow (NPIF)) is an improvement of at least 5%, 10%, 20%, 30%, 40%, 50%, 60%, 70%, 80%, or at least 90%, e.g., relative to the (baseline) level prior to treatment (e.g., in the same subject) or relative to a control (e.g., a control subject or a control population)

For example, to determine whether a reduction (e.g., of a symptom, biomarker or pathogen) or an improvement (e.g. of a test score) has occurred, e.g., upon treatment comprising administering the substantially complete sinonasal microbial composition described herein, the parameter is quantified at baseline (e.g., prior to treatment/administration) and at a time point after administration of the substantially complete sinonasal microbial composition described herein (with or without one or more co-treatments). For example, the parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, or longer, after the initial treatment. In embodiments, the parameter is measured daily (e.g., once or twice per day), weekly, biweekly, or monthly. In other embodiments, the parameter is measured daily, and the mean value determined over the course of a month is compared to baseline. The difference between the value of the parameter at a particular time point following initiation of treatment and the value of the parameter at baseline is used to establish whether there has been an improvement, an increase or decrease, as the case may be, depending on the specific parameter being measured.

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include cleaning of the mucosal lining of the sinuses (at least in part). Cleaning of the mucosal lining of the sinuses may be performed to achieve one or more of the following: a) disrupting the structure of present biofilm, b) decreasing the viability of microbial populations of the sinonasal passages, c) providing space, e.g., on the mucosal lining for administering the substantially complete sinonasal microbial composition in the sinonasal cavities and/or engraftment of one or more microbial taxa (e.g., bacterial strains comprised in the substantially complete sinonasal microbial composition) and subsequent population doubling, d) preventing the formation of new biofilm and/or facilitating the subject's own immune system to prevent the formation of new biofilm. In embodiments, cleaning is performed to allow modification of the resident microbiome in the sinonasal cavities, e.g., by administering the substantially complete sinonasal microbial composition to a subject's sinonasal cavities, e.g., to reduce the built-up or prevent the recurrence of a biofilm that causes a pathology, e.g., a biofilm comprising one or more pathogens, and/or a biofilm comprising one or more microbial taxa that induce inflammation (e.g., pro-inflammatory microbial taxa), that is, e.g., associated with (chronic) sinusitis, (chronic) rhinitis, and/or (chronic) rhinosinusitis (with or without polyps) and exacerbations. Biofilms are a common feature of chronic forms of the diseases (e.g., CRS). Biofilms can line mucosal surfaces and comprise microbes (e.g., bacterial and/or fungal pathogens, including multidrug resistant (bacterial) strains) in a matrix of extracellular polymeric substance (generally composed of extracellular DNA, proteins and polysaccharides) providing adhesion and encasement that can help protect, e.g., bacteria from antibiotic-induced killing and shield them from mucociliary clearance by the sinuses. In embodiments, different microbes, e.g., bacteria and fungi, coexist in biofilms, e.g., of patients with CRS. In embodiments, cleaning includes at least partial removal of biofilms, e.g., reducing the sinonasal microbes comprised in the biofilm by at least 1-log in at least a portion of the sinonasal cavities of the subject, and includes one or more of: disruption of the biofilm's structure, increasing antimicrobials penetration and microbial susceptibility, and/or interrupting the microbial quorum sensing signals, including signals by which microbes in biofilms interact with neighboring cells.

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include modulating the sinonasal microbiome, e.g., reducing the abundance of one or more pathogens (e.g., pathogens associated with or causative of inflammation and/or allergic reaction), reducing the abundance of one or more microbes correlated with symptom severity, reducing the abundance of one or more microbes correlated with inflammation (e.g., microbes eliciting or potentiating an inflammatory host response), and/or modulating the functional composition and output of the resident microbiome and change disease states.

In embodiments, one or more pathogens may reside in in the subject sinonasal microbiota, yet these subjects exhibit no or minor symptomology. In embodiments, subjects with similar pathogens compositions and/or microbial load (e.g., as determined by nucleic acid sequencing) in their niches (e.g., sinonasal niche) exhibit different clinical outcomes. In embodiments, sinonasal mucosal health depends on the local composition of resident microbiota within the niche.

The method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include modulating the sinonasal microbiome, e.g., to supply and/or promote the growth of microbial taxa that are a) associated with healthy sinuses, b) provide mucosal protection, c) provide a hostile niche environment for invasion and colonization of pathogens (e.g., protection against unwanted microbial growth), and/or d) provide a low or anti-inflammatory niche environment (reduction or prevention of mucosal inflammation). In embodiments, the method for treatment described herein, e.g., comprising administering the substantially complete sinonasal microbial composition described herein can be used to modulate (e.g., increase or decrease) microbial (e.g., bacterial) diversity (e.g., alpha diversity) in the subject's microbial niche (e.g., the sinonasal microbiome). In embodiments, the method for treatment described herein, e.g., comprising administering the substantially complete sinonasal microbial composition described herein can be used to modulate (e.g., increase or decrease) the abundance and/or pathogenic behavior of specific members of the microbial community within the niche, including controlling their overgrowth and/or virulence gene expression, as well as controlling perturbations to the local microbiota composition that can contribute to inflammatory disease etiology.

In embodiments, administering the substantially complete sinonasal microbial composition described herein leads to such changes in the resident sinonasal microbiome(s) of a subject exhibiting a (e.g., respiratory tract) inflammatory disease, such as, e.g., (chronic) sinusitis, (chronic) rhinitis, and/or (chronic) rhinosinusitis (with or without polyps), optionally with concomitant diseases such as asthma or other Th2-associated inflammatory diseases. For example, one or more (e.g., at least one, at least two, or at least three) microbial taxa (e.g., bacterial strains) comprised in the substantially complete sinonasal microbial composition described herein (e.g., derived from a healthy donor subject) may engraft or colonize (e.g., transiently or more permanently) in the sinonasal microbial niche of the host recipient subject, who may exhibit an inflammatory (e.g., respiratory tract) disease or condition, such as, e.g., (chronic) sinusitis, (chronic) rhinitis, and/or (chronic) rhinosinusitis (with or without polyps). The one or more (e.g., at least one, at least two, or at least three) microbial taxa (e.g., bacterial strains) comprised in the substantially complete sinonasal microbial composition described herein may successfully engraft or colonize, e.g., by undergoing population doublings and adherence to the mucosal lining and establishment in the niche. In embodiments, such microbial taxa (e.g., bacterial strains) reside in the niche (e.g., nasal and/or sinus microbiome)—and can be detected (e.g., by nucleic acid sequencing)—for more than 1 day, 2, 3, 4, 5, 6 days, more than 1 week, 2, 3, 4, 5, 6, 7, 8 weeks, more than 1 month, 2, 3, 4, 5, 6, 7, 8, 9, 10, 11, or more than 12 months. In embodiments, successful engraftment, e.g., as determined by detection in the recipient subject's microbial niche (e.g., sinonasal microbiome) of one or more taxa comprised in the substantially complete sinonasal microbial composition described herein, e.g., by taking a biological sample, is associated with one or more of reducing the abundance of one or more pathogens (e.g., pathogens associated with or causative of inflammation and/or allergic reaction), reducing the abundance of one or more microbes correlated with symptom severity, reducing the abundance of one or more microbes correlated with inflammation (e.g., microbes eliciting or potentiating an inflammatory host response), and/or modulating the functional composition and output of the resident microbiome and change disease states. A suitable control may be used to determine modulation of the recipient's microbiome and/or successful engraftment/colonization of microbial taxa in the recipient niches. For example, the recipient's microbiome may be analyzed (e.g., by nucleic acid sequencing) prior to administration of the substantially complete sinonasal microbial composition described herein and then again after administration, e.g., 1 day, 2, 3, 4, 5, 6 days, more than 1 week, 2, 3, 4, 5, 6, 7, 8 weeks, more than 1 month, 2, 3, 4, 5, 6, 7, 8, 9, 10, 11, or more than 12 months after administration and the (sequencing) analyses compared to determine changes in the microbiome. In addition, e.g., to determine engraftment, the resident microbiome of the recipient (subject exhibiting the disease) and the microbial taxa comprised in the substantially complete sinonasal microbial composition (derived from a healthy donor subject) are analyzed. The microbiome of the recipient is then re-analyzed, e.g., 1 day, 2, 3, 4, 5, 6 days, more than 1 week, 2, 3, 4, 5, 6, 7, 8 weeks, more than 1 month, 2, 3, 4, 5, 6, 7, 8, 9, 10, 11, or more than 12 months after administration and the (sequencing) analyses compared to determine if any one or more microbial taxa (e.g., strain) originating from the substantially complete sinonasal microbial composition successful engrafted or colonized and continue to reside in the recipient's microbial niche, e.g., the sinonasal microbiome.

In embodiments, the recipient's (subject exhibiting the disease) microbiome in the sinonasal cavities comprises one or more of Streptococcus pneumoniae, Hemophilus influenzae, Moraxella catarrhalis, Staphylococcus aureus (including methicillin-resistant, MRSA); anaerobic bacteria, e.g., Fusobacterium, Prevotella, Porphyromonas, and Peptostreptococcus; and fungi, e.g., Bipolaris, Curvularia, Exserohilum, Alternaria, Drechslera, Helminthosporium, and Fusarium Aspergillus, Penicillium, Cladosporium, and Candida. In embodiments, the recipient's (subject exhibiting the disease) microbiome in the sinonasal cavities comprises one or more potential pathogen, such as, e.g., Corynebacterium tuberculostearicum, Haemophilus influenzae/H. aegyptius, and Staphylococcus, as well as, e.g., Campylobacter spp., Streptococcus pneumoniae, and Streptococcus pyogenes, and M. catarrhalis.

In embodiments, the recipient (subject exhibiting the disease) microbiome's microbial colonization with one or more pathogens is chronic and associated with chronic sinusitis, chronic rhinitis, and/or chronic rhinosinusitis (with or without polyps).

In embodiments, detecting a plurality of microbes in the biological sample includes characterizing the microbiome in the biological sample. In embodiments, characterizing the microbiome in the biological sample includes detecting the number and/or identity of microbial taxa represented by microbes in the biological sample. In embodiments, the microbes are bacterial microbes. In embodiments, the microbes are fungal microbes. In embodiments, the microbes are viral microbes. In embodiments, microbes (e.g., bacteria) may be differentiated at, e.g., the family level, the genus level, the species, level, the sub-species level, the strain level or by any other taxonomic method described herein and otherwise known in the art. In embodiments, the microbial taxa include microbial families, genera, and/or species. In embodiments, the microbial taxa comprise, consist essentially of, or consist of microbial families (e.g., bacterial families). In embodiments, the microbial taxa are microbial families (e.g., bacterial families). In embodiments, the microbial taxa comprise, consist essentially of, or consist of microbial genera (e.g., bacterial genera). In embodiments, the microbial taxa are microbial genera (e.g., bacterial genera). In embodiments, the microbial taxa comprise, consist essentially of, or consist of microbial species (e.g., bacterial species). In embodiments, the microbial taxa are microbial species (e.g., bacterial species). In embodiments, the microbial taxa are bacterial families and/or genera. In embodiments, detecting a plurality of microbes in the biological sample includes amplifying and sequencing 16S rRNA genes of microbes in the sample. In embodiments, a method herein includes determining the expression level of at least one gene in the biological sample.

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include modulating the inflammatory host response. In embodiments, the methods include reducing host inflammation. In embodiments, the host inflammation is spread over a lager anatomical area, e.g., upper airway inflammation, lower airway inflammation. In embodiments, the host inflammation is localized e.g., to the sinonasal cavities. In some embodiments, the severity of inflammation differs at different sites of the body, e.g., between low-grade (e.g., low-grade lower airway inflammation) and high-grade (e.g., high-grade sinonasal inflammation).

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include reducing the inflammatory host response (e.g., sinonasal inflammation and/or upper/lower airway inflammation). In embodiments, the reduction of inflammation is assessed by measuring one or more markers of inflammation (e.g., pro- and/or anti-inflammatory markers) in the subject, including one or more (e.g., 2, 3, 4, 5, 6, 7, 8, 9, 10 or more) of: interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), and interferons (e.g., IFN-γ) or any combination thereof, e.g., compared to a control. In embodiments, the reduction of inflammation is assessed by measuring one or more markers of inflammation (e.g., pro- and/or anti-inflammatory markers) in the subject, including one or more (e.g., 2, 3, 4, 5, 6, 7, 8 or more) of: IFN-γ, TNF-α, IL-4, IL-5, IL-13, ECP, P-gp, IL-17A, and IL-23 or any combination thereof, e.g., compared to a control. In embodiments, the reduction of inflammation is assessed by measuring one or more markers of inflammation (e.g., pro- and/or anti-inflammatory markers) in the subject, including one or more (e.g., 2, 3, 4, 5, 6, 7, 8, 9, 10 or more) of: Occludin, Claudin 2, Mucin 5AC (MUC5AC), Interleukin-4 (IL-4), Interleukin-5 (IL-5), Interleukin-6 (IL-6), Interleukin-8 (IL-8), Interleukin-25 (IL-25), Interleukin-17A (IL-17A), Interleukin-10 (IL-10), Interleukin-1β (IL-1β), Interleukin-33 (IL-33), C-C motif chemokine 11 (CCL11), Thymic stromal lymphopoietin (TSLP), Tumor necrosis factor alpha (TNF-α), Arginase 1 (ARG1), Transforming growth factor beta 1 (TGFβ1), Chloride channel accessory 1 (CLCA1), and/or Interferon gamma (IFN-γ) or any combination thereof, e.g., compared to a control. In embodiments, the reduction of inflammation is assessed by measuring levels of eosinophils and/or eosinophil infiltration to a specific site. In embodiments, inflammation is assessed by measuring levels of Th 1/Th 17-inflammatory mediators. In embodiments, inflammation is assessed by measuring levels of Th 2-inflammatory mediators.

The recipient subject (in need of treatment) or patient receiving the substantially complete sinonasal microbial composition described herein and benefiting from treatment, in some embodiments, exhibits one or more markers of inflammation, such as, e.g., pro-inflammatory interleukins, growth factors, chemokines, interferons or any combination thereof, e.g., associated with (chronic) sinusitis, (chronic) rhinitis, and/or (chronic) rhinosinusitis (with or without polyps), e.g., compared to a control.

The recipient subject (in need of treatment) or patient receiving the substantially complete sinonasal microbial composition described herein and benefiting from treatment, in some embodiments, exhibits one or more markers of Th 1/Th 17-mediated inflammation, e.g., associated with (chronic) sinusitis, (chronic) rhinitis, and/or (chronic) rhinosinusitis (with or without polyps), e.g., compared to a control.

The recipient subject (in need of treatment) or patient receiving the substantially complete sinonasal microbial composition described herein and benefiting from treatment, in some embodiments, exhibits one or more markers of Th 2-mediated inflammation, e.g., associated with (chronic) sinusitis, (chronic) rhinitis, and/or (chronic) rhinosinusitis (with or without polyps) e.g., compared to a control.

The recipient subject (in need of treatment) or patient receiving the substantially complete sinonasal microbial composition described herein and benefiting from treatment, in some embodiments, exhibits one or more markers of (chronic) inflammation, including higher blood leukocyte, monocyte, eosinophil and/or neutrophil counts, modulated (e.g., increased or decreased) neutrophil-to-lymphocyte ratio (NLR), monocyte-to-lymphocyte ratio (MLR), platelet-to-lymphocyte ratio (PLR), C-reactive protein (CRP) and albumin ratio (CAR), eosinophil-to-lymphocyte ratio (ELR), erythrocyte sedimentation rate (ESR) and/or C-reactive protein (CRP) levels, e.g., compared to a control.

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include decreasing one or more markers of inflammation, such as, e.g., pro-inflammatory interleukins, growth factors, chemokines, interferons or any combination thereof, e.g., associated with (chronic) sinusitis, (chronic) rhinitis, and/or (chronic) rhinosinusitis (with or without polyps), e.g., compared to a control.

In embodiments, the method for treatment described herein, e.g., comprising administering the substantially complete sinonasal microbial composition described herein, e.g., to treat (chronic) sinusitis, (chronic) rhinitis, and/or (chronic) rhinosinusitis (with or without polyps) include returning elevated pro-inflammatory marker levels to basal levels in a subject, e.g., compared to a control.

### Detection Methods

In embodiments, detecting a compound (e.g., a metabolite) and/or the expression level thereof includes, but is not limited to, Ultrahigh Performance Liquid Chromatography-Tandem Mass Spectrometry (UPLC-MS/MS), Nuclear magnetic resonance (NMR), Gas Chromatography-Mass Spectrometry (GC-MS), high performance liquid chromatography (HPLC), gas chromatography, liquid chromatography, Mass spectrometry (MS), inductively coupled plasma-mass spectrometry (ICP-MS), accelerator mass spectrometry (AMS), thermal ionization-mass spectrometry (TIMS) and spark source mass spectrometry (SSMS), matrix-assisted laser desorption/ionization (MALDI), and/or MALDI-TOF.

In embodiments, detecting the expression level of a protein includes assaying the level of the compound (e.g., with high-performance liquid chromatography (HPLC), liquid chromatography-mass spectrometry (LC/MS), an enzyme-linked immunosorbent assay (ELISA), protein immunoprecipitation, immune-electrophoresis, protein immunostaining, and/or Western blot) or the level of mRNA that encodes the protein. In embodiments, detecting the expression level of a compound (e.g., a protein) includes lysing a cell. In embodiments, detecting the expression level of a compound includes a polymerase chain reaction (e.g., reverse transcriptase polymerase chain reaction), RNA sequencing, microarray analysis, immunohistochemistry, or flow cytometry.

### Selection of Healthy Donor Subjects

In embodiments, the substantially complete sinonasal microbial composition described herein is obtained from at least one healthy donor subject selected by selection criteria, wherein the selection criteria comprise one or more of (i) the absence of symptoms associated with upper respiratory tract diseases or conditions (e.g., CRS, sinusitis, rhinitis, etc.), (ii) substantial absence of one or more microbial pathogens, (iii) absence of transmittable diseases, (iv) absence of nasal polyps, and/or (v) absence of history of sinonasal or lower airway disease within a predetermined time period. The one or more selections criteria (i)-(v) may be used in isolation or in any combination to select the healthy donor subject.

### Symptoms

The healthy subject may be selected based on the absence of symptoms associated with acute or chronic sinusitis, rhinitis or rhinosinusitis, e.g., CRS. The absence of the symptoms may be used as a criterium alone or in combination with any of the other selection criteria.

Typically, symptoms associated with CRS comprise one or more of nasal congestion/obstruction, facial discomfort (pain/pressure, tenderness and swelling around the patient's eyes, cheeks, nose or forehead), nasal discharge, headache, hyposmia/anosmia, ear pain/pressure, aching in the upper jaw and teeth, cough, sore throat, fatigue, irritability, and nausea.

The symptoms associated with CRS may be absent for at least 6 months, 9 months, 1 year, 2 years, 3 years or longer. In some embodiments, the healthy subject has no history of CRS or other sinonasal or lower airway diseases and has no history of any symptoms associated with such diseases or conditions.

If desired, the symptoms may be assessed by using the SNOT-22 score, wherein a healthy subject has a SNOT-22 score of less than or equal to 7. In addition or alternatively, the symptoms may be assessed using the Lund-Mackay (LM) score or the endoscopic Modified Lund Kennedy (MLK) score. The MLK score is thought to represent an objective measure of disease burden and describes endoscopic findings based on the degree of scarring, oedema, polyposis and discharge noted during an endoscopic exam.

### Microbial Pathogens and Transmittable Diseases

The healthy donor subject may further be selected by the substantial absence of one or more microbial pathogens, alone or in combination with any of the other selection criteria. In embodiments, the substantially complete sinonasal microbial composition obtained from a healthy donor subject is released from a centralized processing facility, if a predetermined level of microbial pathogens (e.g., substantial absence of pathogens in the collected mucosal fluid sample) and a predetermined level of viability and/or quantity of sinonasal microbes, have been obtained or confirmed.

In embodiments, for the methods provided herein, the healthy donor subject is selected by screening for the substantial absence (e.g., local, e.g., within the sinonasal cavities, or serological, e.g., blood, plasma) of one or more microbial pathogens. In some embodiments, the absence of pathogens is confirmed from a subject sample obtained from the sinonasal cavities, e.g., sinonasal or nasal mucus. In some embodiments, the substantial absence of pathogens is confirmed from a serological subject sample, e.g., a blood sample or plasma sample.

In embodiments, the healthy donor subject is selected by screening for the absence of one or more transmittable diseases. In one embodiment, the healthy subject is selected by screening for the absence of MRSA (Methicillin-resistant Staphylococcus aureus). In embodiments, the healthy donor subject is selected by screening for the absence of SARS-CoV-2, e.g., using a nasopharyngeal swab or a sinonasal sample. In embodiments, the healthy donor subject is selected by screening for the absence of Neisseria, e.g., using a nasopharyngeal swab or a sinonasal sample. In embodiments, the healthy donor subject is selected by screening for the absence of influenza. In embodiments, the healthy donor subject is selected by screening for the absence of tuberculosis, e.g., in a sputum sample of the subject. In embodiments, the healthy donor subject is selected by screening, e.g. in a serum sample, for the absence of any one of syphilis, HIV, HepB, HepC, and/or HTLV1/2.

In some embodiments, the healthy donor subject is screened for the commonly found viruses, such as Herpes simplex virus, Varicella Zoster, Cytomegalovirus, or Epstein-Barr virus (EBV). An otherwise healthy donor subject who is a carrier for one or more of Herpes simplex virus, Varicella Zoster, Cytomegalovirus, or EBV, may be selected as a putative donor for a recipient subject that has the same status (e.g., viral presence), e.g., a EBV carrier who is otherwise healthy would be considered a putative donor for an EBV carrier recipient. Such EBV matches of donor and recipient provide for better outcomes than EBV mismatched donor and recipients, and in embodiments, are considered medically acceptable.

### Absence of Nasal Polyps Nasal Polyposis

The healthy donor subject may further be selected by the absence of nasal polyps, alone or in combination with any of the other selection criteria.

Nasal polyps are soft, painless, noncancerous tissue growth on the lining of the nasal or sinonasal cavities, and associated with irritation and inflammation of the lining of nasal and sinonasal cavities in chronic sinusitis, rhinitis and rhinosinusitis.

### History of Sinonasal or Lower Airway Disease

The healthy donor subject may be selected based on the absence of history of sinonasal or lower airway disease (with the exception of the common cold) within the last 1, 2, 3, 4, or 5 years. In one embodiment, the healthy donor subject has no history of sinonasal or lower airway disease within the last 2 years, with the exception of the common cold.

### Substantially Complete Sinonasal Microbial Compositions

Provided herein are substantially complete sinonasal microbial compositions and methods of producing thereof. The methods of producing a substantially complete sinonasal microbial composition include collecting from a healthy donor subject mucosal fluid from the sinonasal cavities comprising sinonasal microbes and preserving the mucosal fluid in a container for the purpose of producing a substantially complete sinonasal microbial composition. The substantially complete sinonasal microbial composition comprises the bacteria, fungi/yeasts and phages including their metabolites and mucous that are found in the sinonasal cavities of the subject.

If desired, the container can be a storage container, e.g., for storing the substantially complete sinonasal microbial composition, e.g., refrigerated or frozen. In embodiments, the container is a single-use tube, e.g., made from polypropylene, a bag or dish. The container optionally is a cryo-vial (e.g., suitable for freezing) or a container suitable for centrifugation or lyophilization. The container is preferably sterile.

In embodiments, the substantially complete sinonasal microbial composition comprises, is derived from, or processed from a biological sample, described herein. In embodiments, the substantially complete sinonasal microbial composition comprises, is derived from, or processed from biological sample comprising sinonasal mucus comprising sinonasal microbes (including, but not limited to, bacteria). The complete sinonasal microbial composition may further comprise phages, yeast and fungi, as well as microbial products such as proteins and metabolites. Furthermore, it may include host derived molecules that are specifically induced by sinonasal microbes (e.g., cytokines, enzymes and antimicrobials). In embodiments, the substantially complete sinonasal microbial composition comprises mucosal fluid derived from, e.g., a nasal saline wash, nasal mucus, or nasal discharge, e.g., obtained from a healthy donor subject. In embodiments, the substantially complete sinonasal microbial composition comprises mucosal fluid derived from, e.g., sinus mucus (e.g., mucus obtained or collected from the surface of a sinus), e.g., obtained from a healthy donor subject. In embodiments, the substantially complete sinonasal microbial composition comprises mucosal fluid derived from, e.g., a brushing including mucus from the sinonasal cavities, e.g., obtained from a healthy donor subject. Methods of obtaining a mucosal fluid from a subject (e.g., a recipient or donor subject) are known in the art. Such methods include swabbing or brushing the sinonasal mucosa, e.g., using anesthetic or endoscopic methods if necessary. Samples can also be obtained using a nasal lavage or spray in sufficient volume to obtain sample from the appropriate location. In embodiments, the substantially complete sinonasal microbial composition comprises a mucosal biopsy, e.g., obtained from a healthy donor subject.

In embodiments, the substantially complete sinonasal microbial composition comprises mucosal fluid obtained from a healthy donor subject comprising sinonasal microbes. In embodiments, the sinonasal microbes are constituents of the sinus microbiome, e.g., the microbial community preferentially residing in the sinus cavities. In embodiments, the sinonasal microbes are constituents of the nasal microbiome, e.g., the microbial community preferentially residing in the nasal cavities. In embodiments, the sinonasal microbes are constituents derived from both the sinus microbiome and the nasal microbiome.

In embodiments, the substantially complete sinonasal microbial composition comprises mucosal fluid obtained from a healthy donor subject comprising a substantially complete community of sinonasal microbes (e.g., comprising substantially complete sinonasal microbiota). For example, in embodiments, the substantially complete sinonasal microbial composition is not obtained by selective culturing (e.g., under culturing conditions that favor specific taxa, e.g., using antimicrobials, specific nutrients or nutrient depletion, temperature, oxygen levels, etc.), and/or does not undergo selective separation/purification steps (e.g., size exclusion, e.g., filtering, centrifugation) to obtain one or more, e.g., less than three, four, or less than five individual taxa (e.g., specific microbial strain(s)), or other substantially reduced preparation of a sinonasal microbiota. In embodiments, the substantially complete sinonasal microbial composition includes compositions that comprise reduced microbiota, e.g., when compared to the biological sample (e.g., mucosal fluid sample), e.g., through incidental loss of microbes, e.g., caused by processing of the sample. However, the reduction in microbes during processing is not substantial, e.g., a substantial amount of the microbiome/microbiota is preserved. In embodiments, less than 1%, 2%, 3%, 5%, 8%0, 1, 15%, 20%, 25%, 30%, 35%, 40%, 45% or less than 50% of constituents of the microbiome are lost during the preparation/processing of a biological sample to obtain a substantially complete sinonasal microbial composition, e.g., when compared to a control, e.g., the biological sample from which the substantially complete sinonasal microbial composition is processed and obtained.

In embodiments, the sinonasal microbes obtained from a healthy donor subject comprise one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas. In embodiments, the substantially complete sinonasal microbial composition comprises one or more of Bifidobacterium, Corynebacterium, Staphylococcus, Streptococcus, Dolosigranulum, and Moraxella. In embodiments, the substantially complete sinonasal microbial composition comprises one or more of Corynebacterium, Staphylococcus, Streptococcus, Haemophilus, Moraxella, and Pseudomonas. In embodiments, the substantially complete sinonasal microbial composition comprises one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas. In embodiments, the sinonasal microbes obtained from a healthy donor subject comprise one or more of the phyla Actinobacteria, Bacteroidetes, Firmicutes, and Proteobacteria, e.g., with representatives of genera Bifidobacterium, Corynebacterium, Staphylococcus, Streptococcus, Dolosigranulum, Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellagioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus and Moraxella. In embodiments, the sinonasal microbes obtained from a healthy donor subject comprise one or more of Corynebacterium, Staphylococcus, Streptococcus, Haemophilus, Moraxella, and Pseudomonas.

Described herein are substantially complete sinonasal microbial composition is obtainable by a method comprising:
a. processing collected mucosal fluid comprising sinonasal microbes from sinonasal cavities of a healthy donor subject in a centralized processing facility,
b. assessing the absence of one or more pathogens,
c. assessing viability and/or quantity of the sinonasal microbes, and
d. releasing the composition comprising the processed mucosal fluid for the aforementioned use if a predetermined level is obtained in step (b) and (c),
   wherein steps (b) and (c) can be performed prior to and/or after the processing in step (a), thereby obtaining the composition.

In embodiments, the method may further comprise one, two, three, four or all of:
a. adding at least one pharmaceutically acceptable diluent, excipient or carrier;
b. adjusting the pH, osmolarity and/or viscosity of the mucosal fluid;
c. adding one or more cryoprotectants (e.g., for freezing) and/or one or more lyoprotectants (e.g., for drying);
d. formulating the processed mucosal fluid into a dosage form comprising a powder, a solid, a semi-solid, or a liquid; and
e. partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU).

### Predetermined Level Absence of Pathogens

Assessing the pathogens that may be comprised in the substantially complete sinonasal microbial composition ensures that the composition is suitable to be administered to exert a beneficial effect on the recipient subject and does not cause an infection in the recipient subject. In embodiments, the method of obtaining the substantially complete sinonasal microbial composition thus comprises a step of assessing the level of pathogens and releasing the composition only if a predetermined level of pathogens is obtained. The predetermined level of pathogens may be the absence of substantially absent level of pathogens. In embodiments, the predetermined level of pathogens allows up to 5%, 4%, 3%, 2%, 1%, or 0.5% pathogens of the microbial composition to be comprised. The predetermined levels of the pathogens are not effective or causative for an infection in a recipient subject. In some embodiments, the pathogens comprise pathobionts, which can be commensal in nature but can also become pathogenic (e.g., can cause or promote disease) when certain genetic and/or environmental conditions are present in a subject. In embodiments, the predetermined level of pathobionts is <30%, <20%, <10%, <5%, <2%, <1%, <0.5%, or <0.1% pathobionts in the substantially complete sinonasal microbial composition.

Predetermined Viability and/or Quantity of Sinonasal Microbes

The method of obtaining the substantially complete sinonasal microbial composition may comprise a step of assessing the viability and/or quantity of sinonasal microbes comprised in the composition, and releasing the composition only if a predetermined level of viability and/or quantity of the sinonasal microbes is obtained. In embodiments, the predetermined viability and/or quantity may be assessed with live/dead staining or by determining colony forming units (CFUs). In embodiments, the predetermined viability is 10³ to 10¹⁵ colony forming units (CFUs), 10⁴ to 10¹¹ CFUs/mL, 10⁵ to 10¹¹ CFUs/mL, or 10⁶ to 10¹¹ CFUs/mL. In embodiments, the predetermined viability is a threshold value of 50%, 60%, 70% or more viable cells in a composition.

Predetermined level of pH

The nasal mucosal pH is a clinical parameter that in subjects with rhinitis, sinusitis or CRS is more alkaline than in healthy subjects (England et al., Nasal pH measurement: a reliable and repeatable parameter, Clin Otolaryngol Allied Sci. 1999 February; 24(1):67-8. doi: 10.1046/j.1365-2273.1999.00223.x). In some embodiments, the nasal mucosa of subjects having rhinitis, sinusitis or CRS is approximately pH 7.2-8.3. While the substantially complete sinonasal microbial composition of healthy donor subjects, e.g., subject without CRS symptoms and having a SNOT-22 score of ≤7, is slightly acidic and has typically a pH of approximately pH 5.5-6.5, the pH may be adjusted to a predetermined level. This may be the case if, e.g., the addition of excipients, or other processing steps resulted in alteration or alkalizing of the pH of the composition. The method of obtaining the substantially complete sinonasal microbial composition may comprise a step of adjusting the pH to a predetermined level, wherein the predetermined level is pH 4.5-7.5, optionally pH 5.0-6.5. In embodiments, the pH is adjusted to 4.5-5.0, 5.0-5.5, 5.5-6.0, 6.0-6.5, 6.5-7.0, or 7.0-7.5. In embodiments, the predetermined level is approximately pH 5.5, 6.0, or 6.5. In some embodiments, the predetermined pH is approximately pH 5.5-6.0. In some embodiments, the pH is adjusted to the predetermined pH level by addition of acids or bases according to routine methods in the art.

### Predetermined Level of Viscosity

Sinonasal fluid is a viscous fluid, wherein the level of viscosity is variable and depends, inter alia, on the rate of mucociliary transport, the water content and the amount of glycoproteins or mucins present in the fluid (Majima et al., Rheologic properties of nasal mucus from patients with chronic sinusitis. American Journal of Rhinology, Volume: 7 issue: 5, page(s): 217-221, Sep. 1, 1993). The viscosity of a fluid can be determined with any known methods in the art, such as a rheometer or viscosimeter. The method of obtaining the substantially complete sinonasal microbial composition may comprise a step of adjusting the viscosity to a predetermined level. This enables that the substantially complete sinonasal microbial composition can be aliquoted or stored in uniform and homogenous units. In some embodiments, the method of obtaining a substantially complete sinonasal microbial composition comprises a step of adjusting the viscosity to a predetermined level of ≤1000 cP, ≤750 cP, or ≤500 cP. In some embodiments, the predetermined level is approximately 1-500 cP, 1-400 cP, 1-300 cP, 1-200 cP, 1-100 cP, 1-50 cP, or 1-25 cP. In some embodiments, the predetermined level is approximately 1-100 cP or 1-50 cP.

### Predetermined Level of Osmolarity

Adjusting the osmolarity of the substantially complete sinonasal microbial composition ensures that the osmolarity and thus fluid homeostasis in the sinonasal cavities is not perturbed (Togias, et al., The osmolarity of nasal secretions increases when inflammatory mediators are released in response to inhalation of cold, dry air. Am Rev Respir Dis. 1988 March; 137(3):625-9. doi: 10.1164/ajrccm/137.3.625). In embodiments, the method of obtaining the substantially complete sinonasal microbial composition may comprise a step of adjusting the osmolarity to a predetermined level. In embodiments, the predetermined level of osmolarity of the substantially complete sinonasal microbial composition is set to 200-500 mOsm/kg, such as 220-480 mOsm/kg, 250-450 mOsm/kg, 300-400 mOsm/kg, or 320-380 mOsm/kg. Osmolarity may be determined by methods known in the art, e.g., using a vapor pressure osmometer; and adjusted by addition of e.g., water to reduce osmolarity, or by addition of an excipient, such as mannitol, to increase osmolarity.

In embodiments, the substantially complete sinonasal microbial composition comprising mucosal fluid derived from, e.g., a healthy donor is processed further, e.g., one or more (two or more, three or more) processing steps may be taken, including: a) adding one or more (pharmaceutically acceptable) excipients or carriers to the mucosal fluid, b) adjusting one or more composition parameters, such as, e.g., pH, osmolarity and/or viscosity, e.g., by adding acids, bases, buffers, and/or salts to the mucosal fluid, c) adding one or more cryoprotectants (e.g., for freezing) and/or lyoprotectant (e.g., for drying) to the mucosal fluid, d) performing filtration (e.g., obtaining a filtrate) and/or centrifugation (e.g., obtaining a supernatant) of the mucosal fluid, e) lyophilizing or spray drying the mucosal fluid, and/or f) performing further processing steps such as, e.g., separating (e.g., obtaining a supernatant of the mucosal fluid); formulating (e.g., into a powder, a solid, a semi-solid, or a liquid, and further formulating into, e.g., an aerosol, spray, foam, irrigation, film, gel, or nasal drops); partitioning (e.g., into discrete units, e.g., with a desired dose, e.g., a desired range of colony-forming units (CFUs)), packaging, labeling, etc.

In embodiments, the methods further comprise one, two, three, four or all of: (a) adding at least one pharmaceutically acceptable diluent, excipient or carrier; (b) adjusting the pH, osmolarity and/or viscosity of the mucosal fluid; (c) adding one or more cryoprotectants (e.g., for freezing) and/or one or more lyoprotectants (e.g., for drying); (d) formulating the processed mucosal fluid into a dosage form comprising a powder, a solid, a semi-solid, or a liquid; and (e) partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU).

In embodiments, the methods of producing a substantially complete sinonasal microbial composition include determining the colony-forming units (CFU) of the mucosal fluid sample derived from a healthy donor and optionally partitioning the sample, e.g., into discrete units, e.g., with a desired dose, e.g., a desired range of colony-forming units (CFUs), e.g., of sinonasal microbes comprised in the substantially complete sinonasal microbial composition. In embodiments, a dose of the substantially complete sinonasal microbial composition may comprise from 10³ to 10¹⁵ CFU. In embodiments, a dose of the substantially complete sinonasal microbial composition may comprise from 10⁴ to 10¹⁵ CFU. In embodiments, a dose of the substantially complete sinonasal microbial composition may comprise from 10⁵ to 10¹⁵ CFU. In embodiments, a dose of the substantially complete sinonasal microbial composition may comprise from 10⁵ to 10¹² CFU. In embodiments, a dose of the substantially complete sinonasal microbial composition may comprise from 10⁶ to 10¹⁵ CFU. In embodiments, a dose of the substantially complete sinonasal microbial composition may comprise from 10⁷ to 10¹⁵ CFU. In embodiments, a dose of the substantially complete sinonasal microbial composition may comprise from 10⁸ to 10¹⁵ CFU. In embodiments, a dose of the substantially complete sinonasal microbial composition may comprise from 10¹ to 10¹⁵ CFU. In embodiments, a dose of the substantially complete sinonasal microbial composition may comprise from 10¹⁰ to 10¹⁵ CFU. In embodiments, a dose of the substantially complete sinonasal microbial composition may comprise from 10¹¹ to 10¹⁵ CFU. In embodiments, a dose of the substantially complete sinonasal microbial composition may comprise from 10¹² to 10¹⁵ CFU. In embodiments, a dose of the substantially complete sinonasal microbial composition may comprise from 10¹³ to 10¹⁵ CFU. In embodiments, a dose of the substantially complete sinonasal microbial composition may comprise from 10¹⁴ to 10¹⁵ CFU. The effective dose of sinonasal microbes may comprise at least 10⁶, 10⁷, 10⁸, 10⁹, 10¹⁰, 10¹¹, 10¹², 10¹³ or 10¹⁴ CFUs.

In some embodiments, a dosage form, e.g., a tablet or liquid, may comprise a single dose of the substantially complete sinonasal microbial composition. In some embodiments, the dosage form, e.g., an aerosol, liquid or gel, may comprise multiple doses of the substantially complete sinonasal microbial composition.

Provided herein are methods of treatment comprising administering to a subject in need thereof an effective dose of the substantially complete sinonasal microbial composition. Amounts effective for this use will depend upon the route of administration, the severity of the condition, and the general state of the patient's health. Single or multiple administrations of the compositions may be administered depending on the dosage and frequency as required and tolerated by the patient.

To determine a therapeutically effective dose, a relatively low dose of the composition can be initially administered to the individual, and the dose can be incrementally increased until the condition of the individual, e.g., the sinonasal environment, begins to improve (e.g., reduced mucin secretion, reduced inflammation, etc.). In embodiments, the initial dose is relatively high, e.g., to establish a colonizing population of (sinonasal-derived) microbes comprised in the substantially complete sinonasal microbial composition in a patient experiencing acute or chronic symptoms. One of skill will appreciate that a number of variables must be considered when determining a therapeutically effective dose. The dose administered to a patient should be sufficient to effect a beneficial therapeutic response in the patient over time. The size of the dose also will be determined by the existence, nature, and extent of any adverse side-effects that accompany the administration of a particular composition (or combination therapy) in a particular patient.

In embodiments, the substantially complete sinonasal microbial composition comprising mucosal fluid derived from, e.g., a healthy donor is processed further, e.g., to formulate the composition with one or more excipients or carriers (e.g., pharmaceutically acceptable excipients or carriers), e.g., suitable for sinonasal administration.

In embodiments, the substantially complete sinonasal microbial composition is processed further, e.g., to formulate the composition into a powder, a solid, a semi-solid, a liquid, sol, gel, foam, or aerosol.

In embodiments, the substantially complete sinonasal microbial composition is processed further, e.g., to formulate the composition into a capsule, a tablet, a suspension, a (degradable) nasal insert, a powder, a cream, an oil, an oil-in-water emulsion, a water-in-oil emulsion, or an aqueous solution.

The nature of the dosage form of the substantially complete sinonasal microbial composition is dictated by the route of administration, anatomy of the target area (e.g., to reach a specific part of the sinonasal cavities), the formulation requirements for the (sinonasal) microbes in the composition (e.g., to maintain microbial viability) and the size of the dose.

Pharmaceutical formulations, or pharmaceutical compositions, comprising the substantially complete sinonasal microbial compositions described herein may also include a pharmaceutically acceptable carrier, e.g., for the purpose of facilitating, assisting, or helping the administration or other delivery of the substantially complete sinonasal microbial composition. Examples include diluents, adjuvants, excipients, water, oils (including petroleum, animal, vegetable or synthetic oils.) Such carriers include particulates such as a tablet or powder, liquids such as an oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic acid, sodium alginate, Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carriers. Still further examples of carriers include sterile diluents such as water, saline solution, physiological saline, Ringer's solution, isotonic sodium chloride, fixed oils such as synthetic mono or diglycerides, polyethylene glycols, glycerin, cyclodextrin, propylene glycol, poloxamers, or other solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

In some embodiments, the pharmaceutically acceptable carrier can comprise a growth medium that can support the growth and/or static existence of one or more of the microbial taxa comprised in the substantially complete sinonasal microbial composition. For example, the pharmaceutical formulation can comprise one or pharmaceutically acceptable carrier to provide a sufficient carbon source for the microbes to grow (e.g., in the sinonasal microbial niches) that are also compatible with the desired route of administration (e.g., intranasal or trans-nasal administration).

The pharmaceutical formulations, or pharmaceutical compositions, may include different (dosage) forms, including solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs. The pharmaceutical formulations may include aerosol formulations (nebulized) to be administered via inhalation. Aerosol formulations can be placed into pressurized acceptable propellants, such as dichlorodifluoromethane, propane, nitrogen, etc. in a pharmaceutically acceptable carrier, e.g., an aqueous carrier. A variety of aqueous carriers can be used, e.g., buffered saline. The formulations can contain pharmaceutically acceptable substances as required to, e.g., approximate physiological conditions such as pH adjusting and buffering agents, for example, sodium acetate, sodium chloride, potassium chloride, calcium chloride, sodium lactate, etc. The aerosol formulation may be administered as a pump aerosol or pulsating aerosol, wherein the pulsating aerosol provides for a particularly effective deposition of the substantially complete sinonasal microbial composition into the sinonasal cavities.

Provided herein are substantially complete sinonasal microbial compositions produced by the method described herein.

Further provided are medical kits that include an applicator configured for delivery (e.g., of a fluid, aerosol, mist or dry powder) to the human sinonasal cavities, and a substantially complete sinonasal microbial composition described herein. In embodiments, the applicator is a mechanical device such as a syringe, a dropper, a squeeze bottle, a swab, a spray/atomizer (including, e.g., a syringe-driven atomizer or a pump-driven atomizer) or other mechanical device. Optionally, the mechanical device is a dosing device, e.g., configured to administer a specific dose or dose range, e.g., a specific CFU count. Optionally, the mechanical device is pre-loaded with a substantially complete sinonasal microbial composition for single use or, alternatively, for multiple use.

In embodiments, the substantially complete sinonasal microbial compositions produced by the method described herein are administered using a suitable surgical device known in the art, e.g., directly into the sinonasal cavities.

In embodiments, the substantially complete sinonasal microbial compositions produced by the method described herein are administered nasally, trans-nasally, or to the sinuses, e.g., using an aerosol, spray, irrigation, or nasal drops.

In embodiments, the substantially complete sinonasal microbial compositions produced by the method described herein are administered to one or more parts of the sinonasal cavities, including, e.g., the turbinates, meatus, middle meatus, osteomeatal complex, frontal sinuses, uncinate process, anterior ethmoid, maxillary ostium, ethmoid infundibulum, and anterior ethmoid cells. In embodiments, the applicator comprised in the medical kit is configured for delivery of the substantially complete sinonasal microbial compositions to one or more parts of the sinonasal cavities, including, e.g., the turbinates, meatus, middle meatus, osteomeatal complex, frontal sinuses, uncinate process, anterior ethmoid, maxillary ostium, ethmoid infundibulum, and anterior ethmoid cells.

In embodiments, the methods of producing a substantially complete sinonasal microbial composition include determining the microbial composition of the mucosal fluid sample derived from a healthy donor, e.g., by nucleic acid sequencing. In embodiments, a sample is rejected—and not processed into a substantially complete sinonasal microbial composition by the methods described herein-, e.g., if one or more microbial pathogens (e.g., bacteria, viruses, and/or fungi) are present (e.g., can be detected) or are present above a predetermined threshold (the threshold being determined, e.g., based on safety data). In embodiments, a mucosal fluid sample obtained from a healthy donor is rejected if methicillin-resistant Staphylococcus aureus (MRSA) is detected above a predetermined threshold.

In embodiments, the methods of producing a substantially complete sinonasal microbial composition include determining the health status of the donor of the mucosal fluid sample. In embodiments, the donor provides health-related information, including health history, particularly concerning existing and previously encountered respiratory diseases, disorders, or conditions, as well as other diseases, particularly transmissible diseases (e.g., sexually transmissible diseases, e.g., gonorrhea, HIV, syphilis; orally or contact transmissible diseases, e.g., hepatitis, influenza, tuberculosis, measles, etc.), and other respiratory pathogens, such as, e.g., rhinovirus, respiratory syncytial virus, and other respiratory viruses, e.g., (severe acute respiratory syndrome) coronaviruses, e.g., SARS-CoV, SARS-CoV-2, etc. Such information may include information routinely gathered for chronic sinusitis/rhinosinusitis patients, such as, e.g., SNOT-20, SNOT-22 score. For example, a healthy donor would have a low SNOT-22 score, e.g., between 0 and 7, e.g. 7, 6, 5, 4, 3, 2, 1, or 0. In embodiments, donors are considered healthy in the absence of one or more of (or all of): a) acute or chronic respiratory tract symptoms, b) history of upper respiratory tract disease, c) pathogens, d) diseases and/or infections that can be transmitted.

In embodiments, further screening involves microbial and/or serological screening, e.g., of the donor's blood, plasma, and sinonasal microbiome. In embodiments, a donor is rejected—and not used to derive a mucous sample for generating the substantially complete sinonasal microbial composition, described herein—if the donor's serological and/or microbiome tests show one or more of the following pathogens over a predetermined threshold: Hepatitis A, Hepatitis B, Hepatitis C, HIV, HTLV 1/2, CMV, Treponema pallidum, Strongyloides, Methicillin-resistant Staphylococcus aureus (MRSA). The predetermined threshold may be determined according to health data from controls. If desired, other bacterial, viral and/or fungal pathogens may be assessed and donors selected (and rejected) accordingly.

The substantially complete sinonasal microbial composition may formulated into a dosage form comprising an effective amount of sinonasal microbes in one or more discrete units, wherein the effective amount is predetermined and effective in reducing the symptoms or the severity of symptoms (e.g., effective in treating) one or more diseases and conditions described herein (e.g., CRS).

### Definitions

As used herein, the term “sinusitis” refers to any inflammatory condition characterized by (mucosal) inflammation of the sinonasal cavities (including the nasal cavities and paranasal sinuses). Acute sinusitis lasts for less than about four weeks. Sub-acute sinusitis may persist for up to about eight to twelve weeks. If symptoms (two or more symptoms of nasal congestion or blockage, anterior or posterior nasal discharge, facial pressure or pain, and reduction or loss of smell) persist beyond 8 to 12 weeks (e.g., for months to years), the sinusitis is labeled chronic. Recurrent sinusitis is characterized by sinusitis episodes that occur three or more times per year. The term “rhinitis” refers to an allergic response, such as to a common allergen or to an environmental irritant. The term “rhinosinusitis” refers to a condition that has symptoms of both rhinitis and sinusitis. Rhinosinusitis includes acute rhinosinusitis and chronic rhinosinusitis. Acute rhinosinusitis can be caused by an unwanted microbial growth, such as a bacterial, viral or fungal pathogen growth, or by a chemical irritation (e.g., cigarette-smoke or chemical fumes). Chronic sinusitis (CS) and chronic rhinosinusitis (CRS) are conditions that last longer than eight to twelve weeks. The underlying causes of acute sinusitis and acute rhinosinusitis may lead to chronic sinusitis or chronic rhinosinusitis if the resulting inflammation persists. Symptoms include at least two of: nasal congestion/obstruction, facial discomfort (pain/pressure, tenderness and swelling around the patient's eyes, cheeks, nose or forehead), nasal discharge, headache, and hyposmia/anosmia. Other symptoms may include: ear pain/pressure, aching in the upper jaw and teeth, cough, sore throat, fatigue, irritability, and nausea. CRS is clinically classified into CRS with nasal polyps and CRS without nasal polyps. CRS with nasal polyps can be divided into eosinophilic and non-eosinophilic according to the degree of eosinophil infiltration (e.g., chronic eosinophilic rhinosinusitis with or without nasal polyps). The degree of eosinophil infiltration in nasal polyp tissue is most closely related to recurrence. Nasal polyposis is characterized by long-term symptoms of nasal obstruction and congestion, reduction in or loss of sense of smell, anterior and posterior rhinorrhea, and facial pain. Many patients with chronic sinusitis have chronic hyperplastic eosinophilic sinusitis, which is characterized by marked inflammation of the sinuses, increased eosinophils and mixed mononuclear cells, and a relative paucity of neutrophils. Some of these patients have one or more of associated nasal polyps, asthma, and aspirin or NSAID sensitivity. Additional subcategories of chronic sinusitis (and chronic rhinosinusitis) include, e.g., superantigen-induced eosinophilic chronic sinusitis (e.g., sinusitis induced by exo- and endotoxins produced by bacteria such as Staphylococcus aureus); allergic fungal sinusitis (e.g., sinusitis induced by fungi such as Aspergillus or Alternaria); non-allergic fungal eosinophilic chronic sinusitis; and aspirin-exacerbated eosinophilic chronic sinusitis.

As used herein, “allergic sinusitis” and “allergic rhinitis” refer to sinusitis and rhinitis, respectively, that occurs when the immune system responds to specific irritants or allergens such as, e.g., plant pollen, mold, dust mites, animal hair, industrial chemicals, certain foods, medicines, and insect venom, and can be seasonal, perennial, or occupational.

As used herein, “non-allergic sinusitis” and “non-allergic rhinitis” refer to sinusitis and rhinitis, respectively, that is not caused by an allergy. Common causes are infection, e.g., (acute) viral infection (cold), and environmental factors, such as, e.g., extreme temperatures, humidity or exposure to noxious fumes.

As used herein, “allergic rhinosinusitis” refers to rhinosinusitis that occurs in response to exposure to one or more allergens.

As used herein, “non-allergic rhinosinusitis” refers to rhinosinusitis caused by factors including, but not limited to: pregnancy; thyroid disorders as a side effect of certain blood pressure and/or topical OTC decongestant medications; structural abnormalities in the nasal septum, structural abnormalities in the nasal filtering structures (turbinates), and/or structural abnormalities in the sinus drainage tracts; nasal polyps; and eosinophils.

“Nasal polyposis” is characterized by long-term symptoms of nasal obstruction and congestion, reduction in or loss of sense of smell, anterior and posterior rhinorrhea, and facial pain caused by the presence of multiple polyps in the upper nasal cavities. Chronic rhinosinusitis with nasal polyps has mainly Th2 characteristics affecting the mucosa of the nose and paranasal sinuses with eosinophilia. Interleukin-5, eosinophil cationic protein (ECP), and eotaxin are typically found in nasal polyps.

The terms “refractory” or “recalcitrant” rhinosinusitis are used interchangeably herein and refer to a form of rhinosinusitis that is treatment-nonresponsive using antimicrobials, corticosteroids and decongestants) and/or sinus surgery. A refractory or recalcitrant form of the condition/disease is thus characterized by one or more of high resistance to treatment, poor therapeutic response, and/or a high recurrence rate of the disease. In some embodiments, refractory or recalcitrant rhinosinusitis is thus characterized by a failure of symptom control despite maximal medical intervention, e.g., conventional medical therapy using corticosteroids, antibiotics and/or surgery. The refractory or recalcitrant form of rhinosinusitis is further characterized by persistent clinically significant symptoms despite the maximal conventional or traditional medical therapy.

The term “predetermined” is used herein to denotes threshold values of (i) levels of a (one or more) pathogen, as desired (e.g., a tolerated maximum amount, e.g., as considered safe for a recipient subject, e.g., as determined by regulatory bodies such as FDA and EMA); (ii) a level of microbial viability and/or quantity, e.g., of desired sinonasal microbes, e.g., a minimum level in a given composition or dosage form, e.g., determined to be required to make up the minimum effective dose for a treatment described herein (e.g., upon processing of the mucosal fluid, a minimum of 50%, 60%, 70% or more of cells remain viable in a composition, or a desired total value of CFU in the composition or dosage from, or a desired total concentration in the composition or dosage from, e.g., as expressed in CFU/ml) in; (iii) a predetermined weight or volume of the composition, e.g., wherein the desired weight or volume is determined by the form, shape, size of the dosage form and/or the desired concentration (e.g., CFU/ml) or total microbial content (e.g., CFU) for an effective dose. In embodiments, the predetermined values are selected to standardize the substantially complete sinonasal microbial compositions, dosage forms and pharmaceutical compositions comprising the same. In embodiments, the standardization (e.g., by using the predetermined values and thresholds described herein and then releasing the composition for use) is required for, e.g., manufacturing under good manufacturing practice (GMP) and/or regulatory approval, e.g., by a regulatory body, such as FDA and EMA, e.g., as a regulated product, such as a product regulated under pharmaceutical or cell and tissue regulations. Other predetermined values described herein can include, e.g., pH, osmolarity and/or viscosity of the compositions as desired, e.g., to improve efficacy, mode and/or ease of administration, etc. Predetermined level absence of pathogens: Assessing the pathogens that may be comprised in the substantially complete sinonasal microbial composition ensures that the composition is suitable to be administered to exert a beneficial effect on the recipient subject, and does not causes an infection in the recipient subject. In embodiments, the method of obtaining the substantially complete sinonasal microbial composition thus comprises a step of assessing the level of pathogens and releasing the composition only if a predetermined level of pathogens is obtained. The predetermined level of pathogens may be the absence of substantially absent level of pathogens. In embodiments, the predetermined level of pathogens allows up to 5%, 4%, 3%, 2%, 1%, or 0.5% pathogens of the microbial composition to be comprised. The predetermined levels of the pathogens are not effective or causative for an infection in a recipient subject. In some embodiments, the pathogens comprise pathobionts, which can be commensal in nature but can also become pathogenic (e.g., can cause or promote disease) when certain genetic and/or environmental conditions are present in a subject. In embodiments, the predetermined level of pathobionts is <30%, <20%, <10%, <5%, <2%, <1%, <0.5%, or <0.1% pathobionts in the substantially complete sinonasal microbial composition. Predetermined viability and/or quantity of sinonasal microbes: The method of obtaining the substantially complete sinonasal microbial composition may comprise a step of assessing the viability and/or quantity of sinonasal microbes comprised in the composition, and releasing the composition only if a predetermined level of viability and/or quantity of the sinonasal microbes is obtained. In embodiments, the predetermined viability and/or quantity may be assessed with live/dead staining or by determining colony forming units (CFUs). In embodiments, the predetermined viability is 10³ to 10¹⁵ colony forming units (CFUs), 10⁴ to 10¹¹ CFUs/mL, 10⁵ to 10¹¹ CFUs/mL, or 10⁶ to 10¹¹ CFUs/mL. In embodiments, the predetermined viability is a threshold value of 50%, 60%, 70% or more viable cells in a composition. Predetermined level of pH: The nasal mucosal pH is a clinical parameter that in subjects with rhinitis, sinusitis or CRS is more alkaline than in healthy subjects (England et al., Nasal pH measurement: a reliable and repeatable parameter, Clin Otolaryngol Allied Sci. 1999 February; 24(1):67-8. doi: 10.1046/j.1365-2273.1999.00223.x). In some embodiments, the nasal mucosa of subjects having rhinitis, sinusitis or CRS is approximately pH 7.2-8.3. While the substantially complete sinonasal microbial composition of healthy donor subjects, e.g., subject without CRS symptoms and having a SNOT-22 score of ≤7, is slightly acidic and has typically a pH of approximately pH 5.5-6.5, the pH may be adjusted to a predetermined level. This may be the case if, e.g., the addition of excipients, or other processing steps resulted in alteration or alkalizing of the pH of the composition. The method of obtaining the substantially complete sinonasal microbial composition may comprise a step of adjusting the pH to a predetermined level, wherein the predetermined level is pH 4.5-7.5, optionally pH 5.0-6.5. In embodiments, the pH is adjusted to 4.5-5.0, 5.0-5.5, 5.5-6.0, 6.0-6.5, 6.5-7.0, or 7.0-7.5. In embodiments, the predetermined level is approximately pH 5.5, 6.0, or 6.5. In some embodiments, the predetermined pH is approximately pH 5.5-6.0. In some embodiments, the pH is adjusted to the predetermined pH level by addition of acids or bases according to routine methods in the art. Predetermined level of viscosity: Sinonasal fluid is a viscous fluid, wherein the level of viscosity is variable and depends, inter alia, on the rate of mucociliary transport, the water content and the amount of glycoproteins or mucins present in the fluid (Majima et al., Rheologic properties of nasal mucus from patients with chronic sinusitis. American Journal of Rhinology, Volume: 7 issue: 5, page(s): 217-221, Sep. 1, 1993). The viscosity of a fluid can be determined with any known methods in the art, such as a rheometer or viscosimeter. The method of obtaining the substantially complete sinonasal microbial composition may comprise a step of adjusting the viscosity to a predetermined level. This enables that the substantially complete sinonasal microbial composition can be aliquoted or stored in uniform and homogenous units. In some embodiments, the method of obtaining a substantially complete sinonasal microbial composition comprises a step of adjusting the viscosity to a predetermined level of ≤1000 cP, ≤750 cP, or ≤500 cP. In some embodiments, the predetermined level is approximately 1-500 cP, 1-400 cP, 1-300 cP, 1-200 cP, 1-100 cP, 1-50 cP, or 1-25 cP. In some embodiments, the predetermined level is approximately 1-100 cP or 1-50 cP. Predetermined level of osmolarity: Adjusting the osmolarity of the substantially complete sinonasal microbial composition ensures that the osmolarity and thus fluid homeostasis in the sinonasal cavities is not perturbed (Togias, et al., The osmolarity of nasal secretions increases when inflammatory mediators are released in response to inhalation of cold, dry air. Am Rev Respir Dis. 1988 March; 137(3):625-9. doi: 10.1164/ajrccm/137.3.625). In embodiments, the method of obtaining the substantially complete sinonasal microbial composition may comprise a step of adjusting the osmolarity to a predetermined level. In embodiments, the predetermined level of osmolarity of the substantially complete sinonasal microbial composition is set to 200-500 mOsm/kg, such as 220-480 mOsm/kg, 250-450 mOsm/kg, 300-400 mOsm/kg, or 320-380 mOsm/kg. Osmolarity may be determined by methods known in the art, e.g., using a vapor pressure osmometer; and adjusted by addition of e.g., water to reduce osmolarity, or by addition of an excipient, such as mannitol, to increase osmolarity.

A “biological sample” or “sample” (and in certain embodiments specifically referred to as “sinonasal sample”) includes, but is not limited to, one or any combination of materials obtained from or derived from a subject or patient including cultures, cells, tissues, blood, plasma, saliva, nasal secretions, cerebrospinal fluid, pleural fluid, milk, lymph, sputum, semen, needle aspirates, and the like. Biological samples may be obtained using any methods known in the art. In embodiments, a biological sample is or includes a bodily fluid such as nasal discharge or mucus. In embodiments, a biological sample is or includes a wash, such as a saline wash, e.g., a nasal saline wash. In embodiments, the biological sample is or includes mucus. In embodiments, the mucus is sinus mucus (e.g., mucus obtained or collected from the surface of a sinus). In embodiments, the biological sample is a sinus brushing including mucus from the surface of a sinus. For example, nasal secretions may be obtained from smears, lavage, swabs, brushes and the like. Such methods include swabbing or brushing the sinonasal mucosa, e.g., using anesthetic or endoscopic methods if necessary. Samples can also be obtained using a nasal lavage or spray in sufficient volume to obtain sample from the appropriate location. In embodiments, a biological sample is or includes tissue, such as nasal or sinus (mucosal) tissue, e.g., a mucosal sample from the sinonasal mucosa (e.g., maxillary sinus, ethmoid, frontal, or sphenoid sinus). In embodiments, a biological sample includes sections of tissues such as biopsy and autopsy samples, and frozen sections taken for histological purposes. In embodiments, a biological sample is a bodily fluid obtained by filtration and/or centrifugation. For example, the biological sample may be a filtrate of e.g., a nasal wash, mucus, nasal discharge, a sinonasal brushing, sputum, phlegm, or saliva, or the supernatant of a centrifuged a nasal wash, mucus, nasal discharge, a sinonasal brushing, sputum, phlegm, or saliva. In embodiments, a filtrate is centrifuged. In embodiments a supernatant is filtered.

In embodiments, the substantially complete sinonasal microbial composition described herein comprises, is derived from, or processed from a biological sample. In embodiments, the substantially complete sinonasal microbial composition comprises, is derived from, or processed from biological sample comprising sinonasal mucus comprising sinonasal microbes including their respective microbial metabolites. The term “microbe” refers to, but is not limited to, bacteria, fungi, and viruses, and sinonasal microbes refer to microbes, including bacteria, fungi and/or fungi resident in sinonasal cavities. The substantially complete sinonasal microbial composition, in embodiments, includes bacteria, viruses, fungi, and microbial metabolites. In embodiments, the substantially complete sinonasal microbial composition further comprises mucus, e.g., sinonasal mucus, and human donor-derived cells and molecules. In embodiments, one or more of mucus, e.g., sinonasal mucus, and human donor-derived cells and molecules is substantially removed. In embodiments, the substantially complete sinonasal microbial composition is isolated, and thus separated, from the host, e.g., a healthy donor subject. The substantially complete sinonasal microbial composition is obtained from a donor subject, e.g., a healthy donor subject, by collecting the mucosal fluid from the sinonasal cavities comprising sinonasal microbes. In embodiments, the substantially complete sinonasal microbial composition further comprises the bacteria, fungi/yeasts and phages including their metabolites and mucus that are found in the sinonasal cavities of the donor subject. In some embodiments, the substantially complete sinonasal microbial composition is diluted, e.g., by adding a diluent, or concentrated, e.g., by removing at least a portion of the mucus or mucosal fluid (e.g., by centrifugation). In embodiments, the substantially complete sinonasal microbial composition is thus obtained and isolated from a healthy donor subject, and optionally further processed, e.g., by addition of diluents, excipients, cryoprotectants, lyoprotectants, and/or analyzed for the substantial absence of one or more pathogens. The substantially complete sinonasal microbial composition lacks or substantially lacks pathogens. As used herein, pathogens may comprise or consist of pathobionts. Pathobionts as used herein are microbes that can be commensal in nature but can also become pathogenic (e.g., can cause or promote disease) when certain genetic and/or environmental conditions are present in a subject, e.g., upon alterations of certain host conditions (e.g., changes in the microbe-host interactions), e.g., changes in a microbial niche (e.g., altered microbiota composition, microbe-microbe interactions), host immune/inflammatory response (e.g., impaired host immune defenses). In embodiments, pathogens comprise or consist of pathobionts.

The substantial lack or absence of pathogens (e.g., bacterial pathogens) refers to the presence of less than 40%, 30%, 20%, 10%, 5%, 2%, 1%, 0.5%, 0.1% of pathogens, preferably less than 10%, 5%, 2%, 1%, 0.5%, 0.1% of (total) pathogens, or for any selected pathogen, preferably less than 1.5%, 1%, 0.5%, 0.1%, 0.01% or less than 0.001% (e.g., determined by relative or total abundance of pathogens in the sample/composition), including one or more of methicillin-resistant Staphylococcus aureus (MRSA). Pathogens, in embodiments, also include other respiratory pathogens, such as, e.g., rhinovirus, respiratory syncytial virus, and other respiratory viruses, e.g., (severe acute respiratory syndrome) coronaviruses, e.g., SARS-CoV, SARS-CoV-2. Pathogens, in embodiments, may also include microbes associated with sexually transmissible diseases, e.g., gonorrhea, HIV, syphilis; orally or contact transmissible diseases, e.g., hepatitis, influenza, tuberculosis, measles, which may or may not be present in the mucosal fluid derived from the donor subject, e.g., in the host-derived material comprised in the mucosal fluid sample. In some embodiments, the substantially complete sinonasal microbial composition is obtained by a method, wherein the composition has a predetermined level of one or more microbial pathogen(s). The mucosal fluid may further be preserved in a container for the purpose of producing a substantially complete sinonasal microbial composition. In embodiments, the composition may be stored, e.g., in a frozen or dried form, e.g., for at least 1, 2, 3, 4, 5, 6, 7 days, or 1, 2, 3, 4, 5, 6 months or at least for 1, 2 or at least 3 years. In some embodiments, a first isolated substantially complete sinonasal microbial composition of a donor subject is combined with a second (and if desired, a third) isolated substantially complete sinonasal microbial composition of the same donor to obtain a pooled substantially complete sinonasal microbial composition. Multiple substantially complete sinonasal microbial compositions of the same donor subject may be pooled to adjust the concentration or quantity of the composition, or to adjust the microbial composition thereof. In embodiments, the substantially complete sinonasal microbial composition is produced or obtainable by the method described herein. In one embodiment, the substantially complete sinonasal microbial composition is a pharmaceutical composition. In embodiments, the substantially complete sinonasal microbial composition comprises sinonasal microbes capable of colonizing the sinonasal cavities of a recipient subject for a time period of at least 1 month, 2 months, 3 months, 4 months, 5 months, 6 months or longer. In embodiments, the substantially complete sinonasal microbial composition further comprises at least one pharmaceutically acceptable carrier, diluent or excipient.

The term “process”, “processed” or “processing” as used herein refers to altering a biological sample of a donor subject, e.g. by adding of a diluent, to obtain a substantially complete sinonasal microbial composition, wherein the substantially complete sinonasal microbial composition retains all or substantially all sinonasal microbes, including bacteria, fungi, viruses and metabolites thereof, present in the sinonasal cavities the biological sample was obtained from. Consequently, an isolated microbial strain, or a combination of several microbial strains, cannot be considered to comprise a substantially complete sinonasal microbial composition, because such isolated strains are not obtained from sinonasal cavities and do not encompass substantially all microbes that colonize the sinonasal cavities. In some embodiments, the processing of the biological sample retains the sinonasal mucus in the substantially complete sinonasal microbial composition. In some embodiments, the substantially complete sinonasal microbial composition comprises at least 70%, 80%, 90%, 95%, 99%, 99.5%, or 99.9% of the microbes colonizing the sinonasal cavities of the donor subject. The processing may further comprise, but is not limited to, assessing the presence of absence of pathogens, assessing the viability and/or quantity of sinonasal microbes, retaining the composition in quarantine, releasing the composition if a predetermined level of the level of pathogens, or the viability is obtained, adding at least one pharmaceutically acceptable diluent, excipient or carrier; adjusting the pH, osmolarity and/or viscosity of the mucosal fluid; adding one or more cryoprotectants (e.g., for freezing) and/or one or more lyoprotectants (e.g., for drying); formulating the processed mucosal fluid into a dosage form comprising a powder, a solid, a semi-solid, or a liquid; and partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU).

The term “centralized processing facility” as used herein refers to a facility which regularly processes a plurality of collected mucosal samples or mucosal fluid (e.g., at least 1, 5, 10, 20, 50, 100) per day over a period of time (e.g., 1 month, 2, 3, 6, 9, 12 months). In embodiments, the centralized processing facility complies with good manufacturing practice (GMP), optionally, wherein the facility certified (e.g., under pharmaceutical or cell and tissue regulations) and/or authorized to produce/manufacture such compositions under conditions recognized as suitable for such products by health and regulatory authorities, such as, e.g., FDA and EMA. The substantially complete sinonasal microbial composition described herein is obtainable by a method comprising a step of processing the collected mucosal sample or mucosal fluid in the centralized processing facility.

The term to “colonize” or “engraft” as used herein refers to microbial growth and expansion in a specific niche, e.g., the sinonasal cavities, into which microbes that have been transferred. This can be transient, e.g., one to a few round(s) of doubling, or permanent, e.g., at least 3, 5, 10, 20 or more rounds of microbial doubling in the specific niche. For example, one or more strains comprised in the substantially complete sinonasal microbial composition described herein derived from a donor subject may successfully engraft or colonize one or more parts of the sinonasal cavities of a recipient subject after administration and continue to reside in the recipient's niche for at least one or multiple doublings.

The term “microbial load” refers to a measure of the total number of microbes (e.g., bacteria, viruses, and/or fungi) in a given environment, e.g., an airway mucosal surface or sinonasal mucosal surface. In embodiments, the microbial load refers to bacterial load. Bacterial load can be expressed as colony forming units (CFU) per ml, per gram of sample or tissue, or per surface area.

The term “microbiome” refers to the community of microbes (including, bacteria, archaea, viruses, and fungi), the microbiota, in a defined microbial niche, e.g., a specific host site or niche, e.g., the sinus cavities or the nasal cavities, referred to as “sinus microbiome” and “nasal microbiome”, respectively, and the genetic content/makeup of the microbiota, e.g., the genomic DNA, RNA, such as ribosomal RNA and messenger RNA, the epigenome, plasmids, and all other types of genetic information. The term “sinonasal microbiome” includes both niches, the sinus cavities and the nasal cavities.

“Microbiota” as used herein refers to the community of microbes that occur (sustainably or transiently) in and on a subject (e.g., a human subject). In some embodiments, microbiota specifically refers to a microbial community in a niche.

The term “microbes” includes archaea, bacteria, viruses, and fungi.

“Modulate the microbiota” or “modulate the microbiome” as used herein refers to changing the state of the microbiota and/or microbiome. Changing the state of the microbiota and/or microbiome may include changing the structure and/or function of the microbiota and/or microbiome. In an embodiment, a change in the structure of the microbiota comprises a change in the abundance of a taxa, e.g., relative to another taxa or relative to what would be observed in the absence of the modulation. Modulation of the microbiome may include a change in a function of the microbiome, such as a change in microbiome gene expression, level of a gene product (e.g., RNA or protein), or metabolic output of the microbiome. Changes in the microbiome may be effected by changes in the microbiota. Functions of the microbiota may also include host pathogen protection, host nutrition, host metabolism and host immune modulation. Modulation of the structure or function of the microbiota and/or microbiome may additionally induce a change in one or more functional pathway of the host (e.g., a change in gene expression, level of a gene product, and/or metabolic output of a host cell or host process) as a result of a change in the microbiota or the function of the microbiome.

“Subject” or “patient,” refer to humans, except where indicated (e.g., non-human (laboratory) animals). A subject can be healthy (e.g., a healthy donor subject). For example, a healthy subject is a subject without predetermined symptoms, e.g., without symptoms associated with sinusitis, rhinitis, rhinosinusitis and/or upper respiratory tract inflammation and/or infection. A subject or patient may have a disease (e.g., a subject or patient is diagnosed with chronic sinusitis (CS) or chronic rhinosinusitis (CRS)); or a subject or patient might be seeking medical advice or treatment (e.g., a subject or patient exhibiting one or more symptoms, such as, e.g., nasal congestion/obstruction, facial discomfort (pain/pressure, tenderness and swelling around the patient's eyes, cheeks, nose or forehead), nasal discharge, headache, hyposmia/anosmia, ear pain/pressure, aching in the upper jaw and teeth, cough, sore throat, fatigue, irritability, and nausea); or a subject or patient is already under medical supervision sand is seeking, e.g., monitoring, adjustment or modification of an existing therapeutic regimen, etc.

As used herein, a “symptom” of a disease includes any clinical or laboratory manifestation associated with the disease and is not limited to what a subject can feel or observe. A “reduction” of a symptom or symptoms means decreasing of the severity or frequency of the symptom(s), or elimination of the symptom(s).

The term “diagnosis” refers to a determination or relative probability that a disease (e.g., sinusitis, rhinitis, rhinosinusitis, chronic rhinosinusitis, nasal polyposis, or other disease, such as unwanted microbial growth (e.g., viral, bacterial, fungal pathogen), asthma, GERD, etc.) is present in the subject. In embodiments, a subject is diagnosed with a disease when the disease has been detected (e.g., with an assay) in a subject. The term “prognosis” refers to a relative probability that a certain future outcome may occur in the subject with respect to a disease state. For example, in the context of the present disclosure, prognosis can refer to the likelihood that an individual will develop a disease (e.g., sinusitis, rhinitis, rhinosinusitis, chronic rhinosinusitis, nasal polyposis, or other disease, such as unwanted microbial growth (e.g., viral, bacterial, fungal pathogen), asthma, GERD, etc.), or the likely severity of the disease (e.g., duration of disease/duration of symptoms). The terms are not intended to be absolute.

A “control” or “standard control” refers to a sample, measurement, or value that serves as a reference, usually a known reference, for comparison to a test sample, measurement, or value. For example, a test sample can be taken from a patient suspected of having a given disease (e.g., sinusitis, rhinitis, rhinosinusitis, chronic rhinosinusitis, nasal polyposis, or other disease, such as unwanted microbial growth (e.g., viral, bacterial, fungal pathogen), asthma, GERD, etc.) and compared to a known normal (non-diseased or healthy) individual (e.g., a standard control subject). A standard control can also represent an average measurement or value gathered from a population of similar individuals (e.g., standard control subjects) that do not have a given disease (e.g., standard control population), e.g., healthy individuals with a similar medical background, age, weight, sex, etc., geographical area (e.g., a country or continent), race or ethnicity (e.g., Caucasian, African, of African descent (e.g., African American), Native American, Asian, or of Asian descent, etc.). In embodiments, a standard control is a proportion, level, or amount (e.g., an average proportion, level, or amount) in a general or healthy population of subjects. In embodiments, a standard control is a proportion, level, or amount (e.g., an average proportion, level, or amount) in a general population of subjects. In embodiments, a standard control is a proportion, level, or amount (e.g., an average proportion, level, or amount) in a healthy population of subjects. In embodiments, a general population of subjects is a general population of subjects without a disease. In embodiments, a general population of subjects is a general population of subjects with a disease. A standard or control value can also be obtained from the same individual, e.g., from an earlier-obtained sample from the patient prior to disease onset or prior to treatment, e.g., by obtaining one or more biological samples and conducting one or more analyses. One of skill will recognize that standard controls can be designed for assessment of any number of parameters (e.g., microbiome sequencing, RNA levels, protein levels, specific cell types (e.g., microbial taxa, inflammatory cells), specific bodily fluids, specific tissues, metabolites, other biomarkers, e.g., pro- and anti-inflammatory markers, etc.).

As used herein, “treating” or “treatment of” a condition, disease or disorder or symptoms associated with a condition, disease or disorder refers to an approach for obtaining beneficial or desired results, including clinical results (e.g., improvement in patient comfort and/or respiratory function, etc.). Beneficial or desired clinical results include alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder, or disease, slowing the progression of the condition, disorder, or disease temporarily, although in some instances, it involves halting the progression of the condition, disorder, or disease permanently. In embodiments, treatment can refer to a 10%, 20%, 30%, 40%, 50%, 60%, 70%, 80%, 90%, or 100% reduction in the severity of an established disease, condition, or symptom of the disease or condition (e.g., (chronic) sinusitis, (chronic) rhinitis, and/or (chronic) rhinosinusitis), e.g., as compared to a control or control level. The effect of treatment can be, e.g., compared to an individual or pool of individuals not receiving a given treatment, or to the same patient prior to, or after cessation of, treatment. The term “treatment” is not absolute and does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

In embodiments, a subject is administered an effective amount of one or more active agents, e.g., compounds and/or microbes, e.g., comprised in the substantially complete sinonasal microbial composition described herein (e.g., therapeutic compositions). The term effective amount is defined as any amount necessary to produce a desired physiologic response (e.g., reduction in one or more symptoms of (chronic) sinusitis, (chronic) rhinitis, and/or (chronic) rhinosinusitis). In embodiments, an effective amount is an amount sufficient to accomplish a stated purpose (e.g., to achieve the effect for which it is administered, including, to treat a disease, kill pathogenic cells, reduce one or more symptoms of a disease or condition such as (chronic) sinusitis, (chronic) rhinitis, and/or (chronic) rhinosinusitis, asthma, or nasal polyposis). Effective amounts and schedules for administering the active agent may be determined empirically by one skilled in the art.

The terms “dose” or “dosage” refer to the amount of an active agent given to an individual at each administration. The dose may refer to microbes in microbial compositions (e.g., a specific dose of substantially complete sinonasal microbial composition described herein), or drug agents, e.g., an antimicrobial or anti-inflammatory agent. Dosage can also be expressed in terms of bacterial concentration, e.g., colony-forming units (CFU). The dose will vary depending on various factors, including frequency of administration; size and tolerance of the individual; severity of the condition; risk of side effects; and the route of administration. One of skill will recognize that the dose can be modified depending on the above factors or based on therapeutic progress. The term “dosage form” refers to the format of the active agent and depends on the route of administration. For example, a dosage form can be in a liquid form for nebulization, e.g., for inhalants, in a tablet or liquid, e.g., for oral delivery, or a saline solution, e.g., for injection, or in a gel, film or ointment for topical administration. The dosage ranges for administration are those large enough to produce the desired effect, e.g., in which one or more symptoms of the disease or disorder are affected (e.g., reduced or delayed). The dosage should not be so large as to cause substantial adverse side effects, such as unwanted cross-reactions, anaphylactic reactions, gastrointestinal issues, and the like. Generally, the dosage will vary with the age, condition, sex, type of disease, the extent of the disease or disorder, route of administration, the state of the immune system, or whether other drugs are included in the regimen, and can be determined by one of skill in the art. The dosage can be adjusted by the individual physician in the event of any contraindications. Dosages can vary and can be administered in one or more dose administrations daily, for one or several days. The exact dose and formulation will depend on the purpose of the treatment and will be ascertainable by one skilled in the art using known techniques.

In embodiments, a compound is administered in a composition that includes a pharmaceutically acceptable excipient or carrier. Such pharmaceutical compositions or pharmaceutical formulations thus comprise the composition of the invention and a pharmaceutically acceptable excipient or carrier. “Pharmaceutically acceptable excipient” and “pharmaceutically acceptable carrier” refer to a substance that aids the administration of an active agent to and absorption by a subject without causing a significant adverse toxicological effect on the patient. Non-limiting examples of pharmaceutically acceptable excipients include water, NaCl, normal saline solutions, sugars (e.g., sucrose, lactose, glucose), binders, fillers, disintegrants, lubricants, coatings, gels, sweeteners, flavors, salt solutions (such as Ringer's solution), alcohols, oils, gelatins, carbohydrates such as amylose or starch, fatty acid esters, hydroxyethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein, e.g., the substantially complete sinonasal microbial composition described herein. For example, the substantially complete sinonasal microbial composition described herein may comprise agents for buffering and preservation in storage, and can include buffers and carriers for appropriate delivery, depending on the route of administration.

As used herein, a “nasal polyp” is an overgrowth of tissue in one or more of the nasal cavities. The condition of having nasal polyps is called “nasal polyposis.” About 80% of nasal polyps are highly edematous and filled with eosinophils. Nasal polyps can also present as fibrous, glandular or cystic. Nasal polyps are typically teardrop-shaped growths that form in the nose and/or sinuses, obstructing the sinuses and nasal passages.

Both terms, “cleaning”, e.g., of the sinonasal cavities, and “removal of biofilm”, e.g., at least partial removal of biofilm in the sinonasal cavities, as used herein, include both physical/mechanical removal of microbes from at least a portion of the sinonasal cavities, such as, e.g., cleansing (e.g., salt washes (e.g., iodine)), mechanical disruption (e.g. vibrating balloon), sinonasal scrubbing, endoscopic removal, and the use of surfactants, mucolytics, etc., as well as sterilization techniques that primarily kill or inhibit the growth of microbes, utilizing, e.g., antimicrobials and antimicrobial photodynamic therapy, or any combination thereof. Antimicrobial photodynamic therapy is described in, e.g., M A Biel et.al., “Antimicrobial Photodynamic Therapy Treatment of Chronic Recurrent Sinusitis Biofilms”, Int Forum Allergy Rhinol. 2011 September-October; 1(5): 329-334. The cleaning or removal of the biofilm may comprise a 1-log reduction, 2-log reduction, or 3-log reduction from the initial quantity of microbes present in the biofilm in the sinonasal cavities. A recipient subject may thus be treated with a physical (e.g., mechanical), chemical or biological agent, or a combination thereof, to reduce the quantity of the sinonasal microbes residing in the sinonasal cavities prior to the administration of the substantially complete sinonasal microbial composition.

“Sinonasal”, as used herein, e.g., with respect to microbes, microbiota, cavities, niches, etc., includes both nasal and (paranasal) sinus, e.g., nasal and sinus microbiome and specific nasal and sinus microbial constituents of their respective niche, e.g., sinus cavities and nasal cavities, together the sinonasal cavities, sinonasal microbes, sinonasal microbiota, and sinonasal microbiome.

A “pathogen” is a microbe (a pathogenic microbe) that can cause a disease, e.g., such as an infection in a subject, e.g., a human, including but not limited to, one or more of methicillin-resistant Staphylococcus aureus (MRSA), sexually transmissible diseases, e.g., gonorrhea, HIV, syphilis; orally or contact transmissible diseases, e.g., hepatitis, influenza, tuberculosis, measles, and other respiratory pathogens, such as, e.g., rhinovirus, respiratory syncytial virus, and other respiratory viruses, e.g., (severe acute respiratory syndrome) coronaviruses, e.g., SARS-CoV, SARS-CoV-2. In certain contexts, pathogens also include microbes that are associated with a disease or condition but for which a (direct) causative relationship has not yet been established. In embodiments, pathogens as used herein comprise or consist of pathobionts. In some embodiments, the substantially complete sinonasal microbial composition comprises no pathogens. In embodiments, the substantially complete sinonasal microbial composition comprises substantially no pathogens. The term “substantially no pathogens” indicates the absence of pathogens in the compositions described herein, or the presence of small quantities, such as up to 5%, 4%, 3%, 2%, 1%, 0.5%, 0.1% or 0.01% of the microbial composition. In embodiments, the substantially complete sinonasal microbial composition of a healthy donor may be released from a processing facility if it satisfies predetermined levels of microbial pathogens, wherein the predetermined level may be less than 30%, 20%, 10%, 5%, 4%, 3%, 2%, 1%, 0.5%, 0.1% or 0.01% pathogens, preferably, wherein the predetermined level may be less than 5%, 4%, 3%, 2%, 1%, 0.5%, 0.1% or 0.01% pathogens. In embodiments, pathogens may include microbes that cause a transmittable disease. “Pathobionts” as used herein are microbes that can be commensal in nature but can also become pathogenic (e.g., can cause or promote disease) when certain genetic and/or environmental conditions are present in a subject, e.g., upon alterations of certain host conditions (e.g., changes in the microbe-host interactions), e.g., changes in a microbial niche (e.g., altered microbiota composition, microbe-microbe interactions), host immune/inflammatory response (e.g., impaired host immune defenses), etc. In some embodiments, pathobionts, (e.g., C. tuberculosearticum or S. aureus) are associated with (and/or are causative of) host inflammation/a host inflammatory response. In some embodiments, pathobionts are associated with (and/or are causative of) an inflammatory disease, e.g., a disease described herein, including a respiratory tract inflammatory disease. In some embodiments, the substantially complete sinonasal microbial composition comprises substantially no pathobionts.

The “substantial” absence of pathobionts may be less than 30%, 20%, 10%, 5%, 2%, 1%, 0.5%, 0.1% pathobionts, preferably less than 10%, 5%, 4%, 3%, 2%, 1%, 0.5%, 0.1% pathobionts in the substantially complete sinonasal microbial composition. In embodiments, the substantially complete sinonasal microbial composition may be released from a processing facility if it satisfies predetermined levels of microbial pathobionts, wherein the predetermined level may be less than 20%, 10%, 5%, 2%, 1%, 0.5%, 0.1% pathobionts.

The term “antimicrobial” (agent) means a therapeutic agent that has one or more of the following activities: antibiotic, antifungal, and/or antiviral. In embodiments, one or more antimicrobial agents may be selected as desired, e.g., to kill or inhibit growth/propagation of bacteria (antibiotic), fungi (antifungal) and/or viruses (antiviral). In embodiments, a single antimicrobial agent is selective for a single class of microbe, e.g., gram-positive or gram-negative bacteria. In embodiments, a single antimicrobial agent is more broadly effective.

“Unwanted microbial growth” as used herein refers to microbial (over)growth, e.g., of microbes in a certain niche, e.g., sinonasal cavities, upper respiratory tract, etc., that is not desired, e.g., because it is associated with or causative of, e.g., pathogenesis, development of disease or increase or worsening of one or more symptoms of a disease, exacerbations, etc. In embodiments, the microbial growth is unwanted because it comprises (over)growth of one or more pathogens, e.g., such as a pathogen that causes a bacterial, fungal, or viral infection. In embodiments, the microbial growth is unwanted because it comprises (over)growth of one or more pathobionts, e.g., such as a pathobiont that causes, increases or sustains an inflammatory reaction by the host. In embodiments, the microbial growth is unwanted because it is associated with an unwanted microbial load (e.g., too much or unchecked microbial growth), e.g., in a niche. In embodiments, the microbial growth is unwanted because it is unchecked (e.g., overgrowth), e.g., not sufficiently surveilled and/or curtailed by the host's immune system.

“Ecological niche” or simply “niche” refers to the (defined) ecological space which an organism or group of organisms occupies, such as, e.g., the nasal cavities, the sinus cavities, the sinonasal cavities, the lungs, etc.). In some embodiments, niche specifically refers to a (human host) space that microbes occupy.

The term “cavity” or “cavities” are used interchangeably herein, e.g. in the context of nasal or sinonasal cavities, and refers to an air-filled space that is connected with the nostrils (nasal cavity) and the air-filled spaces that surround the nasal cavity (paranasal sinuses). The term can refer to one side or both side of the face. The paranasal sinuses drain into the nasal cavity. The term “sinonasal cavities” refers to the combination of nasal and sinus cavities.

The term “viability” as used herein refers to the capacity of microbes to reproduce or multiply. The viability of sinonasal microbes comprised in the substantially complete sinonasal microbial composition may be assessed by methods of the art, e.g., live/dead bacterial viability tests (live/dead viability stain), determining cell proliferation, metabolic activity, PCR of microbe components or measuring cell death of the microbes in the sample. In some embodiments, the substantially complete sinonasal microbial composition maintains or preserves at least 10%, 20%, 30%, 40%, 50%, 60%, 70% or a greater degree of viability of the microbes comprised in the donor subject mucosal fluid. In some embodiments, the viability is determined shortly after the sinonasal fluid is collected. In some embodiments, the viability is determined prior to storing the sinonasal fluid or the substantially complete sinonasal microbial composition in a centralized processing facility. In some embodiments, the viability is determined after removing the sinonasal fluid or the substantially complete sinonasal microbial composition from storage and/or quarantine from the centralized processing facility.

The term “quantity” as used herein refers to the number or concentration of microbes comprised in a substantially complete sinonasal microbial composition, which may be determined by methods known in the art, and include, but are not limited to serial dilution and determining CFU/mL, determining the optical density, plate counting, turbidimetric analysis or under a counting chamber. The absolute quantity of the sinobacterial microbes comprised in any sample may be calculated based on the sample volume and the concentration of the microbes (CFU/mL).

### EXAMPLES

### Example 1: Chronic Rhinosinusitis (CRS) Clinical Study

The aim of the study was to estimate any adverse reactions and determine therapeutic efficacy of the substantially complete sinonasal microbial composition comprising sinonasal microbiota on the treatment of CRS.

### Donor Subject Selection Criteria

Suitable donors of a substantially complete sinonasal microbial composition comprising sinonasal microbiota were selected by the following criteria: absence of CRS symptoms (e.g., nasal or sinonasal discharge, nasal or sinonasal congestion, facial tenderness, postnasal drainage, reduced sense of smell and/or taste), and the absence of transmittable pathogens (syphilis, HIV, hepatitis B/C, HTLV 1 and 2, herpes simplex virus, varicella zoster virus, cytomegalovirus, Epstein-Barr virus, M. tuberculosis, Neisseria meningitidis, methicillin-resistant Staphylococcus aureus).

### Patient (Recipient Subject) Eligibility Criteria

All patients were required to meet the following criteria: confirmed active chronic rhinosinusitis (CRS) without polyps, (e.g., verified by endoscopy or CT scan); at least two nasal symptoms, one of which was nasal obstruction or discolored discharge; no nasal polyposis; no antibiotic treatment in the four weeks leading up the onset of trial; no immune deficiency other than low levels of mannose-binding-lectin (which would not affect immune capability of the patient). All patients had recalcitrant disease after undergoing sinus surgery.

### Study Cohort

The study population (n=22) was aged 23-83 (mean age 55) and comprised 18 women and 7 men (3 participants did not complete the study) that satisfied all the above criteria.

### Treatment

Participants received an antibiotic pre-treatment prior to receiving the sinonasal microbial composition. The pre-treatment consisted of administration of Augmentin (875 mg amoxicillin and 125 mg clavulanate), three times a day for 13 days. Alternatively, for patients allergic to Augmentin, the pre-treatment consisted of 500 mg clarithromycin twice a day for 13 days. After completion of the antibiotic pre-treatment, patients received the substantially complete sinonasal microbial composition daily for five consecutive days. The symptoms of CRS patients were assessed at 3 months after the fifth treatment by determining SNOT-22 symptom scores and bacterial abundance and diversity.

### Results

### Reduction of CRS Symptoms (SNOT-22 Score)

The Sinonasal Outcome Test 22 (SNOT-22) score is a symptom-based score used to evaluate treatment outcomes in CRS patients, wherein the score is inversely proportional to the severity of symptoms. Generally, a reduction of the SNOT-22 score is thus a reflection of a reduction of CRS symptoms and therapeutic effect. Moreover, participants were considered to be “responders” if a reduction of ≥9 points in the SNOT-22 score was observed 3 months after completion of receiving the final dose, based on the previously defined Minimal Clinically Important Difference (MCID) (Chowdhury et al., Investigating the minimal clinically important difference for SNOT-22 symptom domains in surgically managed chronic rhinosinusitis. Int. Forum Allergy Rhinol. 2017 December; 7(12):1149-1155. Doi:10,1002/alr.22028). The median baseline of the SNOT-22 score at the beginning of the treatment was 56.5.

The short-term antibiotic pre-treatment course alone failed to provide symptomatic relieve and no clinically relevant symptomatic reduction was observed after 90 days (data not shown). This is characteristic for recalcitrant CRS, which is generally resistant to treatment using antibiotics or surgery.

The SNOT-22 score was statistically significantly reduced by 38% after treatment to a median value of 35. (FIG. 1). Not all participants demonstrated a clinically significant improvement, which was defined as a reduction of the SNOT-22 score of ≥9 points. Participants that showed such a clinically significant improvement were labelled “responders”, whereas participants that only showed a mild reduction of <9 points or even a slight increase on the SNOT-22 scale were labelled “non-responders”.

The individual SNOT-22 scores of all participants at baseline (prior to treatment) and 3 months after completion of the treatment are shown in FIG. 2, wherein the baseline value and follow up value for each participant are each indicated circles connected by a line. Responders (16 out of 22 participants) are indicated by solid circles and solid lines, wherein non-responders (6 out of 22) are indicated by open circles and dotted lines. 72.2% of all participants were categorized as responders demonstrating a clinically significant reduction compared to baseline (FIG. 2), wherein the average SNOT-22 score in the responders was reduced by half (54.5%) (FIG. 3).

### Increase of Sinonasal Microbial Diversity

The sinonasal microbiota is a large and diverse microbial community that inhabits the sinonasal cavities. It is believed that the balance of microbes within this microbial community confers protective properties and imbalance within this community, e.g., a dysbiotic sinonasal microbiome, may lead to pathogenic overgrowth or disease. Interestingly, increased microbial diversity in the sinonasal cavities had been previously observed in healthy people with no history of sinus disease compared to patients with CRS. The microbial diversity can be quantified by the Shannon Index, which is a quantitative measure of the diversity within a microbial community, reflecting how many different types of microbial taxa (such as species) are present in a dataset (e.g., a sinonasal microbiota sample) and further taking phylogenetic relations into account. The Shannon Index thus provides a statistical representation of biodiversity.

The Shannon Index of the study participants prior to treatment (“baseline”) was scattered over a relatively wide range. After treatment with the substantially complete sinonasal microbial composition, the mean Shannon Index increased significantly and was less dispersed (FIG. 4), indicative of a greater degree of diversity of the sinonasal microbiome.

The data of this clinical study indicate that the substantially complete sinonasal microbial composition comprising sinonasal microbiota is suitable for treating CRS or ameliorating symptoms of CRS.

### Example 2: Cross-Over Study Using Probiotic Formulation

The aim of this cross-over study was to determine whether a probiotic formulation comprising a limited number of microbes could induce the same or similar therapeutic effects as observed with the substantially complete sinonasal microbial composition demonstrated in Example 1. The study was of a randomized, double-blinded, crossover, and sham-controlled design.

### Patient Eligibility Criteria

All patients were required to meet the following criteria: confirmed active chronic rhinosinusitis (CRS) without polyps, (e.g., verified by endoscopy or CT scan); at least two nasal symptoms, one of which was nasal obstruction or discolored discharge; no nasal polyposis; no antibiotic treatment in the four weeks leading up the onset of trial; no immune deficiency other than low levels of mannose-binding-lectin.

### Study Cohort

The study population (n=21) was aged 21-80 (mean age 58) and comprised 10 women and 11 men that satisfied all the above criteria.

### Probiotic Formulation

The probiotic formulation comprised 13 Lactobacillus species isolated from the honeybee Apis mellifera (10¹¹ CFU/mL) comprising Lactobacillus apinorum Fhon13N, Lactobacillus mellifer Bin4N, Lactobacillus mellis Hon2N, Lactobacillus kimbladii Hma2N, Lactobacillus melliventris Hma8N, Lactobacillus helsingborgensis Bma5N, Lactobacillus kullabergensis Biut2N, Lactobacillus kunkeei Fhon2N, Lactobacillus apis Hma11N, Bifidobacterium asteroides Bin2N, Bifidobacterium coryneforme Bma6N, Bifidobacterium Bin7N, and Bifidobacterium Hma3N.

The probiotic formulation was obtained by combining 10⁸ CFU/mL of each Lactobacillus species with Swedish sterilized heather honey (93%) and bee-pollen in water (7%). Honey and bee pollen act as nutrients and stimulate the microbiota to produce bioactive metabolites. This formulation was diluted with sterile water (5 g/10 mL), incubated for 48 hours at 35° C., resulting in a spray formulation with a Lactobacilli cell count of approximately 10¹¹ CFU/mL.

### Treatment

The study participants (n=21) were randomly divided into two groups A and B, wherein group A first received the placebo (saline) and after a wash-out period, received the active intervention in the form of the probiotic formulation comprising 13 lactobacillus species. Conversely, group B first received the probiotic formulation, and after a wash-out period, received the placebo as the second leg of the study.

In detail, for the first leg of the study, the participants received daily treatments with saline (control) or the probiotic formulation (active intervention) for two weeks. This was followed by a 4-week wash-out period. Subsequently, in the second leg of the study, the groups A and B switched the treatment regimen, wherein saline or the probiotic formulation were administered daily for 2 weeks again. The total length of the cross-over study was 8 weeks.

### Results

### Reduction of CRS Symptoms (SNOT-22 Score)

The SNOT-22 score was obtained from group A and B prior to treatment and after the cross-over treatments were concluded 8 weeks after beginning the first treatment.

The median SNOT-22 score for all observations prior to administration of the probiotic formulation and the placebo was 45.5 (IQR 23.0-58.5). The scores after two weeks' administration of the formulation and placebo were 38.0 (IQR 28.0-68.5) and 34.0 (IQR 17-55), respectively. No statistically significant differences were seen between groups that had received the probiotic formulation or the placebo, and no statistically significant difference was observed after administration of the probiotic formulation or the placebo (Martensson et al., Clinical efficacy of a topical lactic acid bacterial microbiome in chronic rhinosinusitis: A randomized controlled trial. Laryngoscope Investig Otolaryngol. 2017 Nov. 8; 2(6):410-416).

Examples 1 and 2 suggest that the substantially complete sinonasal microbial composition comprising sinonasal microbiota described herein, which represents the transplantation of the entire ecosystem present in the substantially complete sinonasal microbial composition, may be advantageous over approaches utilizing a limited number of isolated or defined bacteria, such as lactobacilli.

### Example 3: Murine Disease Model

Sinonasal microbes derived from the sinonasal cavities of healthy subjects are demonstrated to be efficacious in a murine model of sinonasal infection. A substantially complete sinonasal microbial composition comprising sinonasal microbiota is dosed after inoculation of the mice with a pathobiont (e.g., C. tuberculosearticum or S. aureus), either pre-treated (arm 1) or not (arm 2) with antibiotics. Control animals receive treatment with sterile normal saline only after inoculation. Mice treated with substantially complete sinonasal microbial composition show one or more of the following: reduced pathobiont burden in the sinonasal cavities, decreased goblet cell hyperplasia and/or mucus hypersecretion.

### Example 4: In Vitro Assays

Sinonasal microbes derived from the sinonasal cavities of healthy donor subjects are demonstrated to a) inhibit growth of pathogens and/or b) reduce the pro-inflammatory nature of pathogens in culture. Cultured isolates and/or conditioned medium derived from those isolates provide inhibition of growth of select pathogens (e.g., S. aureus) in culture. Further, these isolates are found to be adherent to airway epithelial cells in culture. When grown together with pathogens in the presence of airway epithelial cells, there is an overall reduction in the production of pro-inflammatory cytokines.

### Example 5: Optimizing Collection of Substantially Complete Sinonasal Microbial Compositions

The goal of this study is to determine the parameters to optimize the sinonal mucosal fluid collection (e.g., the overall quantity, quality or to optimize the bacterial composition, bacterial viability) from a healthy donor subject. To this end, three different modes of collecting sinonasal microbiota from healthy donor subjects are compared. In particular, fluid volume, abundance of total microbiota, relative abundance of subpopulations of microbes (e.g., abundance of particular desired microbial species), viability of the recovered microbes dependent on different collection modes, participant experience, and the potential for serial self-collection are assessed.

### Donor Subject Eligibility Criteria

Suitable donor subjects will be selected based on the following criteria: between 18 to 65 years of age, SNOT-22 score of ≤7, no nasal polyposis, no history of sinonasal or lower airway disease within the last 2 years other than the common cold, no antibiotic treatment within the last 12 weeks, no endoscopy findings indicating sinonasal disease, no nasal rinses during the last month, no unwillingness to cease out-of-study nasal rinses for the duration of the study, and absence of transmittable pathogens and pathobionts (MRSA, syphilis, HIV, hepatitis B/C, HTLV 1 and 2, herpes simplex virus, varicella zoster virus, cytomegalovirus, Epstein-Barr virus, M tuberculosis, Neisseria meningitidis, methicillin-resistant Staphylococcus aureus).

### Study Cohort

The study population will include 18 study subjects aged 18-65, who is divided into 6 groups, each group consisting of 3 subjects.

### Collection Methods

Method 1: Subjects use a Nasaline® nasal rinsing device to introduce 15 ml of saline into their nasal cavity, hold the saline in place with their heads down for 5 mins, then expel the fluid into a kidney dish.

Method 2: Subjects use a Nasaline® nasal rinsing device to introduce 15 ml of saline into their nasal cavity, hold the saline in place with their heads down for 2 mins, then expel the fluid into a kidney dish.

Method 3: Subjects use a parisinus nebulisation device to inhale 5 ml of saline into the nasal cavity, following by blowing out their noses into a kidney dish.

### Study Visits

All subjects have approximately five study visits, each of which will include a sample collection process of one of the 3 methods described above. The visits comprise: Visit 1 (Day 1): Information and signed consent; SNOT-22 questionnaire to confirm health and absence of symptoms; clinical examination including nasal endoscopy; testing for MRSA using an E-swab from the middle meatus; baseline nasal lavage using Method 1; randomization to group A, B or C.

Visit 2 (Day 8): A sinonasal microbiome sample from the nasal cavities of the trial subject is collected using any one of methods 1, 2 or 3 depending on the assigned trial group. The trial subject's experience of the sampling method is recorded.

Visit 3 (Day 15), Visit 4 (Day 22), and Visit 5 (Day 23) are identical to visit 2.

### Analysis

The following parameters are assessed in each group: overall sinonasal fluid volume; Sinonasal fluid mass; physical characteristics (e.g., consistency, pH, osmolarity); culture-based assessments for microbial cell count and concentration (e.g., aerobic and anaerobic CFU assays); assessment of viability of sinonasal microbes; culture-independent assessment for microbial cell count (e.g., qPCR for 16S sequencing); sequence-based assessments for metagenomic assessments of microbial composition and biodiversity.

### ENUMERATED EMBODIMENTS

1. A method of reducing or preventing the number of repeated sinus medical therapy and/or sinus surgery in a subject in need of such treatment comprising: optionally, cleaning the sinonasal cavities, and administering a substantially complete sinonasal microbial composition to the sinonasal cavities, thereby reducing the number of repeated sinus medical therapy and/or sinus surgery.
2. A method of treating sinusitis in a subject in need of such treatment comprising: optionally, cleaning the sinonasal cavities, and administering a substantially complete sinonasal microbial composition to the sinonasal cavities, thereby treating sinusitis.
3. A method of treating rhinitis in a subject in need of such treatment comprising: optionally, cleaning the sinonasal cavities, and administering a substantially complete sinonasal microbial composition to the sinonasal cavities, thereby treating rhinitis.
4. The method of any one of embodiments 1-3, wherein cleaning the sinonasal cavities comprises at least partial removal of one or more of: debris/solids, mucus and/or microbes from at least a portion of the sinonasal cavities.
4A. The method of embodiment 4, wherein cleaning the sinonasal cavities comprises at least partial removal of a biofilm comprising sinonasal microbes from at least a portion of the sinonasal cavities.
5. The method of any one of embodiments 1-4, wherein cleaning the sinonasal cavities comprises one or more of: cleansing/rinsing, sinonasal scrubbing, endoscopic/surgical removal, antimicrobial photodynamic therapy, antimicrobials, surfactants, mucolytics, salt washes (e.g., iodine) or a combination thereof.
6. The method of any one of embodiments 1-5, wherein the substantially complete sinonasal microbial composition comprises an effective dose of sinonasal microbes derived from the sinonasal cavities of a healthy subject (e.g., a sinonasal sample donor).
7. The method of embodiment 6, wherein the substantially complete sinonasal microbial composition comprises an effective dose of sinonasal microbes derived from the sinus microbiota (e.g., the microbial community preferentially residing in the sinus cavities).
8. The method of embodiment 6, wherein the substantially complete sinonasal microbial composition comprises an effective dose of sinonasal microbes derived from the nasal microbiota (e.g., the microbial community preferentially residing in the nasal cavities).
9. The method of embodiment 6, wherein the substantially complete sinonasal microbial composition comprises an effective dose of sinonasal microbes derived from both the sinus microbiota and the nasal microbiota.
10. The method of any one of embodiments 1-9, wherein the substantially complete sinonasal microbial composition comprises nasal saline wash, nasal mucus, or nasal discharge.
11. The method of any one of embodiments 1-9, wherein the substantially complete sinonasal microbial composition comprises sinonasal mucus (e.g., a brushing comprising mucus from the sinonasal cavities).
12. The method of any one of embodiments 1-11, wherein the healthy subject is selected by screening for the absence (e.g., local, e.g., within the sinonasal cavities, or serological, e.g., blood, plasma) of one or more microbial pathogens or pathobionts.
13. The method of any one of embodiments 1-12, wherein the healthy subject is selected by screening for the absence of one or more transmittable diseases (e.g., influenza, tuberculosis).
14. The method of any one of embodiments 1-13, wherein the treatment reduces inflammation.
15. The method of embodiment 14, wherein the inflammation is local (e.g., the sinonasal cavities) or affecting larger areas of the body (e.g., upper airway inflammation).
16. The method of embodiment 14 or 15, wherein the reduction of inflammation is assessed by measuring one or more markers of inflammation (e.g., pro- and/or anti-inflammatory markers) in the subject.
16A. The method of embodiment 16, wherein the one or more markers of inflammation comprises one or more of: interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), interferons (e.g., IFN-7), and/or Periostin, eosinophil cationic protein (ECP), P-glycoprotein (P-gp), metabolites (e.g. metabolites involved in arginine metabolism (e.g., arginine, glutamine, and aspartic acid, citrulline, urea, and proline; metabolites involved in purine metabolism (e.g., xanthosine, xanthine, guanosine, adenine, GMP, adenosine, and guanine; metabolites involved in tryptophan and NAD+ metabolism), chemokines, monokines.
16B. The method of embodiment 16 or 16A, wherein the one or more markers of inflammation comprise one or more of IL-8, RANTES, and TGF-beta.
17. The method of any one of embodiments 1, 2, 4-16B, wherein the subject is diagnosed with sinusitis.
18. The method of embodiment 17, wherein the sinusitis comprises:
   a. acute sinusitis
   b. sub-acute sinusitis
   c. chronic sinusitis
   d. allergic sinusitis, and
   e. non-allergic sinusitis.
19. The method of any one of embodiments 1, 3-16B, wherein the subject is diagnosed with rhinitis.
20. The method of embodiment 17, wherein the rhinitis comprises:
   a. allergic rhinitis
   b. non-allergic rhinitis, and
   c. rhinosinusitis (including allergic rhinosinusitis and non-allergic rhinosinusitis).
21. The method of any one of embodiments 1-20, wherein the subject is diagnosed with chronic sinusitis (CS) or chronic rhinosinusitis (CRS) with or without polyps.
22. The method of embodiment 21, wherein the subject exhibits refractory or recalcitrant sinusitis or rhinosinusitis.
23. The method of any one of embodiments 1-22, wherein the subject exhibits one or more of:
   a. asthma,
   b. methicillin resistant Staphylococcus aureus (MRSA) infection,
   c. Samter's triad/polyposis (Aspirin Exacerbated Respiratory Disease (AERD)), and/or
   d. sinonasal sarcoidosis.
24. The method of any one of embodiments 1-23, wherein treatment progression is assessed by one or more of: (a) Sino Nasal Outcome Test (e.g., SNOT-20 or SNOT-22) score; (b) nasal congestion/obstruction assessment, comprising one or more of: including anterior rhinorrhea (runny nose), posterior rhinorrhea (post nasal drip) and loss of sense of smell; (c) number of nightly awakenings; (d) visual analog score (VAS); (e) asthma control questionnaire (ACQ5) score; (f) nasal peak inspiratory flow (NPIF); (g) smell test (University of Pennsylvania Smell Identification Test (UPSIT)); (h) assessment of one or more physiological parameters (e.g., by nasal endoscopy or CT scan); (i) Lund-Mackay Score; and (j) volumetric measurements of at least one part of the sinonasal cavities.
25. The method of any one of embodiments 1-24, further comprising administering a co-therapeutic treatment comprising one or more of: antimicrobials (antibiotic, anti-fungal, anti-viral), anti-inflammatory agents/corticosteroids, anti-septic (e.g., iodine), mucolytic, antihistamine medication, decongestant, or a vasoconstrictor.
26. A method of producing a substantially complete sinonasal microbial composition comprising:
   a. collecting from a healthy donor subject mucosal fluid from the sinonasal cavities comprising sinonasal microbes, and
   b. preserving the mucosal fluid in a container, optionally for storage (e.g., refrigerated or frozen),
   for the purpose of producing a substantially complete sinonasal microbial composition.
27. The method of embodiment 26, wherein the mucosal fluid comprises nasal saline wash, nasal mucus, or nasal discharge.
28. The method of embodiment 26, wherein the mucosal fluid comprises sinonasal mucus (e.g., a brushing comprising mucus from the sinonasal cavities).
29. The method of any one of embodiments 26-28, wherein the sinonasal microbes are constituents of the sinus microbiota (e.g., the microbial community preferentially residing in the sinus cavities).
30. The method of any one of embodiments 26-28, wherein the sinonasal microbes are constituents of the nasal microbiota (e.g., the microbial community preferentially residing in the nasal cavities).
31. The method of any one of embodiments 26-28, wherein the sinonasal microbes are constituents of both the sinus microbiota and the nasal microbiota.
32. The method of any one of embodiments 26-31, wherein the healthy subject is selected by screening for the absence (e.g., local, e.g., within the sinonasal cavities, or serological, e.g., blood, plasma) of one or more microbial pathogens or pathobionts.
33. The method of any one of embodiments 26-32, wherein the healthy subject is selected by screening for the absence of one or more transmittable diseases (e.g., influenza, tuberculosis).
34. The method of any one of embodiments 26-33, wherein one or more (pharmaceutically acceptable) excipients or carriers are added to the mucosal fluid or the mucosal fluid is otherwise further processed.
35. The method of embodiment 34, wherein one or more of: pH, osmolarity and/or viscosity are adjusted, e.g., by adding acids, bases, buffers, and/or salts to the mucosal fluid.
36. The method of embodiment 34 or 35, wherein a cryoprotectant or lyoprotectant is added to the mucosal fluid.
37. The method of any one of embodiments 26-36, wherein the mucosal fluid is lyophilized or spray dried.
38. A substantially complete sinonasal microbial composition produced by the method of any one of embodiments 26-37.
39. A medical kit comprising:
   a. an applicator configured for delivery (e.g., of a fluid, mist or dry powder) to the human sinonasal cavities, and
   b. the substantially complete sinonasal microbial composition of embodiment 38.

1. A substantially complete sinonasal microbial composition for use in a method of reducing symptoms or the severity of symptoms associated with chronic rhinosinusitis (CRS) in a recipient subject in need thereof, the method comprising administering an effective amount of the composition to sinonasal cavities of the recipient subject, thereby reducing symptoms or the severity of symptoms associated with CRS;
wherein the composition is formulated into a dosage form;
wherein the dosage form comprises an effective amount of the composition in one or more discrete units, wherein the effective amount is predetermined and effective in reducing the symptoms or the severity of symptoms associated with CRS;
wherein said composition comprises sinonasal microbes of one or more of Actinobacteria, Bacteroidetes, Firmicutes or Proteobacteria,
wherein the composition is obtainable by a method comprising:
e. processing collected mucosal fluid comprising sinonasal microbes from sinonasal cavities of a healthy donor subject in a centralized processing facility,
f. assessing the absence of one or more pathogens,
g. assessing viability and/or quantity of the sinonasal microbes, and
h. releasing the composition comprising the processed mucosal fluid for the aforementioned use if a predetermined level is obtained in step (b) and (c),
wherein steps (b) and (c) can be performed prior to and/or after the processing in step (a) thereby obtaining the composition.

2. The composition for use of claim 1, wherein reducing the symptoms or the severity of symptoms associated with CRS comprises treating CRS.

3. The composition for use of claim 1 or 2, wherein the recipient subject has nasal or sinonasal polyps.

4. The composition for use of claim 1 or 2, wherein the recipient subject has no nasal or sinonasal polyps.

5. The composition for use of any one of claims 1-4, wherein the recipient subject exhibits refractory or recalcitrant rhinosinusitis.

6. The composition for use of any one of claims 1-5, wherein the recipient subject exhibits one or more of:
a. asthma,
b. methicillin resistant Staphylococcus aureus (MRSA) infection,
c. Samter's triad/polyposis (Aspirin Exacerbated Respiratory Disease (AERD)), and/or
d. sinonasal sarcoidosis.

7. The composition for use of any one of claims 1-6, wherein the recipient subject exhibits one or more of gastroesophageal reflux disease (GERD), immunodeficiency, bronchitis and pneumonia.

8. A substantially complete sinonasal microbial composition for use in a method of treating the sinonasal cavities of a recipient subject in need of treatment exhibiting an adverse condition affecting said sinonasal cavities, the method comprising administering an effective amount of the composition to sinonasal cavities of the recipient subject, thereby treating the adverse condition;
wherein the composition is formulated into a dosage form;
wherein the dosage form comprises an effective amount of the composition in one or more discrete units, wherein the effective amount is predetermined and effective in treating the adverse condition;
wherein said composition comprises sinonasal microbes of one or more of Actinobacteria, Bacteroidetes, Firmicutes or Proteobacteria,
wherein the composition is obtainable by a method comprising:
e. processing collected mucosal fluid comprising sinonasal microbes from sinonasal cavities of a healthy donor subject in a centralized processing facility,
f. assessing the absence of one or more pathogens,
g. assessing viability and/or quantity of the sinonasal microbes, and
h. releasing the composition comprising the processed mucosal fluid for the aforementioned use if a predetermined level is obtained in step (b) and (c),
wherein steps (b) and (c) can be performed prior to and/or after the processing in step (a),
thereby obtaining the composition.

9. A substantially complete sinonasal microbial composition for use in a method of reducing the number of repeated sinus medical therapy and/or sinus surgery in a recipient subject in need of such treatment, the method comprising administering an effective amount of the composition to sinonasal cavities of the recipient subject, thereby reducing the number of repeated sinus medical therapy and/or sinus surgery;
wherein the composition is formulated into a dosage form;
wherein the dosage form comprises an effective amount of the composition in one or more discrete units, wherein the effective amount is predetermined and effective in reducing the number of repeated sinus medical therapy and/or sinus surgery;
wherein said composition comprises sinonasal microbes of one or more of Actinobacteria, Bacteroidetes, Firmicutes or Proteobacteria,
wherein the composition is obtainable by a method comprising:
e. processing collected mucosal fluid comprising sinonasal microbes from sinonasal cavities of a healthy donor subject in a centralized processing facility,
f. assessing the absence of one or more pathogens,
g. assessing viability and/or quantity of the sinonasal microbes, and
h. releasing the composition comprising the processed mucosal fluid for the aforementioned use if a predetermined level is obtained in step (b) and (c),
wherein steps (b) and (c) can be performed prior to and/or after the processing in step (a),
thereby obtaining the composition.

10. The composition for use of claim 8 or 9, wherein the recipient subject is diagnosed with sinusitis.

11. The composition for use of claim 10, wherein the sinusitis is
a. acute sinusitis
b. sub-acute sinusitis, or
c. chronic sinusitis.

12. The composition for use of claim 10 or 11, wherein the sinusitis is
a. allergic sinusitis, or
b. non-allergic sinusitis.

13. The composition for use of claim 8 or 9, wherein the recipient subject is diagnosed with rhinitis.

14. The composition for use of claim 13, wherein the rhinitis is:
a. allergic rhinitis,
b. non-allergic rhinitis,
c. allergic rhinitis rhinosinusitis, or
d. non-allergic rhinosinusitis.

15. The composition for use of any one of claim 8 or 9, wherein the recipient subject is diagnosed with chronic sinusitis (CS) or chronic rhinosinusitis (CRS).

16. The composition for use of any one of claims 8-15, wherein the recipient subject has nasal or sinonasal polyps.

17. The composition for use of any one of claims 8-15, wherein the recipient subject has no nasal or sinonasal polyps.

18. The composition for use of any one of claims 8-17, wherein the recipient subject exhibits refractory or recalcitrant sinusitis or rhinosinusitis.

19. The composition for use of any one of claims 8-18, wherein the recipient subject exhibits one or more of:
a. asthma,
b. methicillin resistant Staphylococcus aureus (MRSA) infection,
c. Samter's triad/polyposis (Aspirin Exacerbated Respiratory Disease (AERD)), and/or
d. sinonasal sarcoidosis.

20. The composition for use of any one of claims 8-19, wherein the recipient subject exhibits one or more of gastroesophageal reflux disease (GERD), immunodeficiency, bronchitis and pneumonia.

21. The composition for use of any one of claims 1-20, wherein the method of obtaining the composition further comprises one, two, three, four or all of:
f. adding at least one pharmaceutically acceptable diluent, excipient or carrier;
g. adjusting the pH, osmolarity and/or viscosity of the mucosal fluid;
h. adding one or more cryoprotectants (e.g., for freezing) and/or one or more lyoprotectants (e.g., for drying);
i. formulating the processed mucosal fluid into a dosage form comprising a powder, a solid, a semi-solid, or a liquid; and
j. partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU).

22. The composition for use of claim 1-21, wherein the method of obtaining the composition further comprises storing the composition for at least 24 hours or at least 48 hours prior to administration to the recipient subject.

23. The composition for use of any one of claims 1-22, wherein the composition is frozen or dried (e.g., lyophilized) prior to administration to the recipient subject.

24. The composition for use of any one of claims 1-23, wherein the dosage from comprises a composition that is frozen or dried (e.g., lyophilized).

25. The composition for use of claim 23 or 24, wherein the composition or dosage form is reconstituted or transitioned to a liquid or aerosolized form prior to administration to the recipient subject.

26. The composition for use of any one of claims 1-25, wherein administering to the recipient subject is performed using a syringe, catheter, nasal dropper, e.g., for nasal irrigation, spray, nebulizer, or aerosolizer, optionally a pump aerosol or a pulsating aerosol.

27. The composition for use of any one of claims 1-26, wherein collecting mucosal fluid comprising sinonasal microbes from sinonasal cavities of a healthy donor subject comprises obtaining the mucosal fluid by rinsing the nasal cavities of the healthy donor subject with a pharmaceutically acceptable rinsing solution.

28. The composition for use of any one of claims 1-27, wherein processing the collected mucosal fluid comprises reducing the volume of the mucosal fluid, optionally by at least 25% or at least 50%.

29. The composition for use of any one of claims 1-28, wherein the composition further comprises one or more of: nasal mucus, nasal saline wash and nasal discharge, optionally, wherein the composition comprises sinonasal mucus (e.g., a brushing comprising mucus from the sinonasal cavities).

30. The composition for use of any one of claims 1-29, wherein the healthy donor subject donates multiple mucosal fluid samples for administration to different recipient subjects.

31. The composition for use of any one of claims 1-30, wherein the composition is obtained from a donor that is not a family member of the recipient subject and/or is not known to the recipient subject.

32. The composition for use of any one of claims 1-31, wherein said substantially complete sinonasal microbial composition comprises one or more of Bifidobacterium, Corynebacterium, Staphylococcus, Streptococcus, Dolosigranulum, Moraxella, Haemophilus, Moraxella, or Pseudomonas.

33. The composition for use of any one of claims 1-32, wherein the composition further comprises a pharmaceutically acceptable diluent, excipient or carrier.

34. The composition for use of claim 33, wherein the composition comprises one or more of a polymer, a carbon source, a mucoadhesive agent, a cryoprotectant, a lyoprotectant, a pH modifier and/or a buffering agent.

35. The composition for use of any one of claims 1-34, wherein said composition is administered nasally, trans-nasally, or to the sinuses.

36. The composition for use of any one of claims 1-35, the method further comprising cleaning the sinonasal cavities of the recipient subject prior to administration of the substantially complete sinonasal microbial composition, wherein cleaning the sinonasal cavities comprises at least a partial removal of one or more of: debris/solids, mucus and/or microbes from at least a portion of the sinonasal cavities.

37. The composition for use of claim 36, wherein cleaning the sinonasal cavities comprises at least a 1-log reduction of the sinonasal microbes comprised in the biofilm of at least a portion of the sinonasal cavities of the recipient subject.

38. The composition for use of claim 36, wherein cleaning the sinonasal cavities comprises a 1-log reduction, 2-log reduction, or 3-log reduction from the initial quantity of microbes present in the biofilm in the sinonasal cavities of the recipient subject.

39. The composition for use of any one of claims 36-38, wherein cleaning the sinonasal cavities comprises one or more of: cleansing/rinsing, pressurized cleansing/rinsing, mechanical disruption of the biofilm (e.g. vibrating balloon), sinonasal scrubbing, salt washes, endoscopic/surgical removal, antimicrobial photodynamic therapy, antimicrobials (e.g., antibiotics), surfactants, mucolytics, salt washes (e.g., iodine) or a combination thereof.

40. The composition for use of any one of claims 1-39, wherein the dosage form comprises an effective dose of sinonasal microbes derived from sinonasal cavities of one healthy donor subject (e.g., a mucosal fluid sample donor).

41. The composition for use of any one of claims 11-40, wherein the dosage form comprises a single or multiple effective doses of sinonasal microbes, wherein the effective dose of sinonasal microbes comprises about 10⁵ to 10¹² colony forming units (CFUs), e.g. about 10⁶-10¹² CFUs, about 10⁷-10¹² CFUs, about 10⁸-10¹² CFUs, about 10⁹-10¹² CFUs, about 10¹⁰-10¹² CFUs, or about 10¹¹-10¹² CFUs, and/or is effective in ameliorating the symptoms associated with (e.g., treating) acute sinusitis, rhinitis or rhinosinusitis or chronic sinusitis, rhinitis or rhinosinusitis.

42. The composition for use of any one of claims 1-40, wherein the effective dose of sinonasal microbes comprises at least 10⁵, 10⁶, 10⁷, 10⁸, 10⁹, 10¹⁰, 10¹¹, or 10¹² CFU.

43. The composition for use of any one of claims 1-42, wherein the effective dose of sinonasal microbes is derived from the sinus microbiota (e.g., the microbial community preferentially residing in the sinus cavities).

44. The composition for use of any one of claims 1-42, wherein the effective dose of sinonasal microbes is derived from the nasal microbiota (e.g., the microbial community preferentially residing in the nasal cavities).

45. The composition for use of any one of claims 1-42, wherein the effective dose of sinonasal microbes is derived from both the sinus and nasal microbiota.

46. The composition for use of any one of claims 1-45, wherein the healthy donor subject is selected by screening for the absence (e.g., local, e.g., within the sinonasal cavities, or serological, e.g., blood, plasma) of one or more microbial pathogens.

47. The composition for use of any one of claims 1-46, wherein the healthy donor subject is selected by screening for the absence of one or more transmittable diseases (e.g., influenza, tuberculosis).

48. The composition for use of any one of claims 1-47, wherein administration of the substantially complete sinonasal microbial composition to the recipient subject reduces inflammation.

49. The composition for use of claim 48, wherein the inflammation is local (e.g., the sinonasal cavities) or affecting larger areas of the body (e.g., upper airway inflammation).

50. The composition for use of claim 48 or 49, wherein the reduction of inflammation is assessed by measuring one or more markers of inflammation (e.g., pro- and/or anti-inflammatory markers) in the subject, comprising one or more of: interleukins (e.g., IL-1, IL-4, IL-5, IL-6, IL-8, IL-10, IL-13, IL-17 (e.g., IL-17A), IL-23, IL-25, IL-33), TNFs (e.g., TNF-α), TGFs (e.g., TGF-β), chemokines (e.g., CXCL-12 and CXCL-13), interferons (e.g., IFN-γ), and/or Periostin, eosinophil cationic protein (ECP), P-glycoprotein (P-gp).

51. The composition for use of claim 48 or 49, wherein the one or more markers of inflammation comprise one or more of IL-8, RANTES, and TGF-beta.

52. The composition for use of any one of claims 1-51, wherein the reduction of symptoms or the severity of symptoms and progression of treatment is assessed by the improvement of one or more of:
(a) Sino Nasal Outcome Test (e.g., SNOT-20 or SNOT-22) score;
(b) nasal congestion/obstruction assessment, comprising one or more of: including anterior rhinorrhea (runny nose), posterior rhinorrhea (post nasal drip) and loss of sense of smell;
(c) number of nightly awakenings;
(d) visual analog score (VAS);
(e) asthma control questionnaire (ACQ5) score;
(f) nasal peak inspiratory flow (NPIF);
(g) smell test (University of Pennsylvania Smell Identification Test (UPSIT));
(h) assessment of one or more physiological parameters (e.g., by nasal endoscopy or CT scan);
(i) Lund-Mackay Score;
(j) Modified Lund-Kennedy score; and
(k) volumetric measurements of at least one part of the sinonasal cavities.

53. The composition for use of claim 52, wherein the improvement of the score or test results of one or more of (a)-(k) are maintained for at least 1 week, 2 weeks, 3 weeks, 1 month, 2 months, 4 months, 6 months, 8 months, 10 months, 1 year, 2 years, 3 years, 5 years, 10 years, or more than 10 years.

54. The composition for use of claim 52, wherein the improvement of the score or test results of one or more of (a)-(k) are maintained for at least 2 months, 4 months, 6 months, 8 months, 10 months, 1 year, 2 years, 3 years, 5 years, 10 years, or more than 10 years.

55. The composition for use of claim 52, wherein the improvement of the score or test results of one or more of (a)-(k) are maintained for at least 1 year, 2 years, 3 years, 5 years, 10 years, or more than 10 years.

56. The composition for use of any one of claim 52-55, wherein the treatment progression is determined or monitored by a reduction of the SNOT-22 score.

57. The composition for use of claim 56, wherein the SNOT-22 score is reduced by at least 9 points, such as at least 10, 15, 20, 25, 30 or more SNOT-22 points from baseline (e.g., prior to administration of the composition).

58. The composition for use of any one of claims 1 to 57, wherein the method further comprises administering to the recipient subject a co-therapeutic treatment comprising one or more of: antimicrobial (e.g., antibiotic, anti-fungal, anti-viral), anti-inflammatory agent/corticosteroid, anti-septic (e.g., iodine), mucolytic, antihistamine medication, decongestant, vasoconstrictor, or a chelating agent medication, or a biofilm-disrupting treatment (e.g. a biofilm disrupting wash, mechanical disruption (e.g. vibrating balloon), cleansing (e.g., salt washes (e.g., iodine)), sinonasal scrubbing, endoscopic removal, and the use of surfactants, mucolytics, etc., as well as sterilization techniques that primarily kill or inhibit the growth of microbes, utilizing, e.g., antimicrobials and antimicrobial photodynamic therapy, or any combination thereof).

59. The composition for use of claim 58, wherein the co-therapeutic treatment is an antimicrobial comprising amoxicillin, other penicillins, cephalosporins, amoxicillin/clavulanate potassium, clarithromycin, beta-lactam, a cephalosporin, a lincosamide, a macrolide, a tetracycline, a sulfa drug (e.g., compound), mupirocin, oxacillin, flucloxacillin, cefazolin, cephalothin, cephalexin, erythromycin, doxycycline, or minocycline.

60. The composition for use of any of claims 1 to 59, wherein the sinonasal microbes (e.g., bacteria) comprised in the complete sinonasal microbial composition are capable of stably engrafting (e.g., colonizing) the sinonasal niche of the subject for a time period of at least 1 month, 2, 3, 4, 5, 6, 7, 8, 9, 10, 11, or more than 12 months.

61. The composition for use of any one of claims 1 to 60, wherein the composition is administered to the subject daily or twice daily.

62. The composition for use of any one of claims 1 to 61, wherein the composition is administered for at least two consecutive days, three consecutive days, four consecutive days, or five consecutive days.

63. The composition for use of any one of claims 1 to 62, wherein the recipient subject prior to treatment displays one or more symptoms selected from the group consisting of nasal congestion/obstruction, facial discomfort/pain or pressure, tenderness and swelling around the patient's eyes, cheeks, nose or forehead, nasal discharge, headache, hyposmia, anosmia, ear pain/pressure, aching in the upper jaw and teeth, cough, sore throat, fatigue, irritability, and nausea, and further wherein the severity or occurrence of the one or more symptoms are reduced after the administration of the composition.

64. The composition for use of claim 63, wherein the severity or occurrence of the one or more symptoms are reduced after the administration of the composition for at least 3 months, 6, months, 9 months, 12 months, 18 months, 24 months, or 36 months.

65. A method of producing a substantially complete sinonasal microbial composition in a centralized processing facility comprising:
e. receiving and processing collected mucosal fluid comprising sinonasal microbes from sinonasal cavities of a healthy donor subject,
f. assessing the absence of one or more pathogens,
g. assessing viability and/or quantity of the sinonasal microbes, and
h. releasing a composition comprising the processed mucosal fluid if a predetermined level is obtained in step (b) and (c), optionally, discarding the composition if a predetermined level in step (b) and (c) is not obtained,
wherein steps (b) and (c) can be performed prior to (e.g., with the mucosal fluid sample) and/or after the processing in step (a) (e.g., with the composition), e.g., by removing a portion of the mucosal fluid sample or composition for testing (e.g., nucleic acid sequencing),
thereby obtaining the composition.

66. The method of claim 65, wherein the composition is a pharmaceutical composition.

67. The method of claim 65 or 66 further comprising one, two, three, four, five or all of:
a. adding at least one pharmaceutically acceptable diluent, excipient or carrier (e.g., to create a diluted mucosal fluid sample);
b. adjusting the pH, osmolarity and/or viscosity of the mucosal fluid;
c. adding one or more cryoprotectants (e.g., for freezing) and/or one or more lyoprotectants (e.g., for drying);
d. formulating the processed mucosal fluid into a dosage form comprising a powder, a solid, a semi-solid, or a liquid;
e. partitioning the mucosal fluid into discrete units, each unit comprising an effective dose of sinonasal microbes, wherein the effective dose of sinonasal microbes comprises about 10³ to 10¹⁵ colony forming units (CFU) or 10⁵ to 10¹² colony forming units (CFU); and
f. preserving the mucosal fluid in a container, optionally for storage (e.g., refrigerated, frozen or dried).

68. The method of any one of claims 65-67 further comprising:
g. storing the refrigerated, frozen or dried mucosal fluid sample or processed composition under quarantine,
h. holding the refrigerated, frozen or dried mucosal fluid sample or processed composition under quarantine until any completion of any combination of (a) testing the donor to exclude the substantial presence of one or more transmissible pathogens, (b) confirming the composition and viability of the microbes comprised in the mucosal fluid sample, or (c) further confirming the health of the donor by a plurality of post-screening tests;
i. standardizing the cell count and/or the quantity or concentration of the sinonasal microbes comprised in the composition, optionally by adding an inert filler; and
j. releasing the refrigerated, frozen or dried mucosal fluid sample or processed composition from quarantine to define the substantially complete sinonasal microbial composition.

69. The method of any one of claims 65-68, wherein the substantially complete sinonasal microbial composition maintains or preserves at least 50%, 55%, 60%, 65%, 70%, 75%, 80%, 85%, 90%, 92%, 95%, 97%, 98%, or at least 99% of the constituents of the microbiome comprised in the donor subject mucosal fluid.

70. The method of any one of claims 65-69, wherein the substantially complete sinonasal microbial composition maintains or preserves at least 10%, 20%, 30%, 40%, 50%, 60%, 70% or a greater degree of viability of the microbes comprised in the donor subject mucosal fluid.

71. The method of any one of claims 65-70 further comprising storing the composition for at least 24 hours or at least 48 hours prior to releasing the composition for use.

72. The method of any one of claims 65-71, wherein the composition is frozen or dried (e.g., lyophilized).

73. The method of any one of claims 65-72, wherein collecting mucosal fluid comprising sinonasal microbes from sinonasal cavities of a healthy donor subject comprises obtaining the mucosal fluid by rinsing of the nasal cavities of the healthy donor subject with a pharmaceutically acceptable rinsing solution.

74. The method of any one of claims 65-73, wherein processing the collected mucosal fluid comprises reducing the volume of the mucosal fluid, optionally by at least 25% or at least 50%.

75. The method of any one of claims 65-74, wherein the composition further comprises one or more of: nasal mucus, nasal saline wash and nasal discharge, optionally, wherein the composition comprises sinonasal mucus (e.g., a brushing comprising mucus from the sinonasal cavities).

76. The method of any one of claims 65-75, wherein the healthy donor subject donates multiple mucosal fluid samples.

77. The method of any one of claims 65-76, wherein the composition further comprises a pharmaceutically acceptable diluent, excipient or carrier.

78. The method of any one of claims 65-77, wherein the composition comprises one or more of a polymer, a carbon source, a mucoadhesive agent, a cryoprotectant, a lyoprotectant, a pH modifier and/or a buffering agent.

79. The method of any one of claims 65-78, wherein the sinonasal microbes are constituents of the sinus microbiota (e.g., the microbial community preferentially residing in the sinus cavities).

80. The method of any one of claims 65-78, wherein the sinonasal microbes are constituents of the nasal microbiota (e.g., the microbial community preferentially residing in the nasal cavities).

81. The method of any one of claims 65-78, wherein the sinonasal microbes are constituents of both the sinus microbiota and the nasal microbiota.

82. The method of any one of claims 65-81, wherein the healthy donor subject is selected by screening for the absence (e.g., local, e.g., within the sinonasal cavities, or serological, e.g., blood, plasma) of one or more microbial pathogens.

83. The method of any one of claims 65-82, wherein the healthy donor subject is selected by screening for the absence of one or more transmittable diseases (e.g., influenza, tuberculosis).

84. The method of claim 82 or 83, wherein the one or more microbial pathogens or transmittable diseases comprise methicillin-resistant Staphylococcus aureus (MRSA), sexually transmissible diseases, e.g., gonorrhea, HIV, syphilis; orally or contact transmissible diseases, e.g., hepatitis, influenza, tuberculosis, measles, and other respiratory pathogens, such as, e.g., rhinovirus, respiratory syncytial virus, and other respiratory viruses, e.g., (severe acute respiratory syndrome) coronaviruses, e.g., SARS-CoV, SARS-CoV-2.

85. The method of any one of claims 65-83, wherein the complete sinonasal microbial composition is not obtained by selective culturing (e.g., under culturing conditions that favor specific taxa, e.g., using antimicrobials, specific nutrients or nutrient depletion, temperature, oxygen levels, etc.).

86. The method of any one of claims 65 to 85, wherein the complete sinonasal microbial composition comprises one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas.

87. The method of any one of claims 65 to 85, wherein the substantially complete sinonasal microbial composition comprises one or more of Bifidobacterium, Corynebacterium, Staphylococcus, Streptococcus, Dolosigranulum, and Moraxella.

88. The method of any one of claims 65 to 85, wherein the substantially complete sinonasal microbial composition comprises one or more of Corynebacterium, Staphylococcus, Streptococcus, Haemophilus, Moraxella, and Pseudomonas.

89. An isolated substantially complete sinonasal microbial composition produced or obtainable by the method of any one of claims 65-88.

90. The substantially complete sinonasal microbial composition of claim 89, wherein the composition is a pharmaceutical composition.

91. The substantially complete sinonasal microbial composition of claim 89 or 90, comprising one or more sinonasal microbes of the phyla Actinobacteria, Bacteroidetes, Firmicutes, and Proteobacteria, preferably wherein said composition comprises one or more of Bifidobacterium, Corynebacterium, Staphylococcus, Streptococcus, Dolosigranulum, Moraxella, Haemophilus, Moraxella, or Pseudomonas.

92. The substantially complete sinonasal microbial composition of claim 89 or 90, wherein said substantially complete sinonasal microbial composition comprises one or more of Bifidobacterium, Corynebacterium, Staphylococcus, Streptococcus, Dolosigranulum, Moraxella, Haemophilus, Moraxella, Pseudomonas, Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellagioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus or Lentilactobacillus.

93. The substantially complete sinonasal microbial composition of any one of claims 89 to 92, wherein the composition comprises sinonasal microbes capable of colonizing the sinonasal cavities of a recipient subject for a time period of at least 1 month, 2 months, 3 months, 4 months, 5 months, 6 months or longer.

94. The substantially complete sinonasal microbial composition of any one of claims 89 to 93, wherein the composition further comprises at least one pharmaceutically acceptable carrier, diluent or excipient.

95. A dosage form formulated for sinonasal administration comprising a substantially complete sinonasal microbial composition of any one of claims 1-64, wherein the substantially complete sinonasal microbial composition is produced or obtainable by the method of any one of claims 65-85.

96. A dosage form formulated for sinonasal administration comprising the substantially complete sinonasal microbial composition of any one of claims 89 to 94.

97. The dosage form of claim 95 or 96 comprising a comprising a substantially complete sinonasal microbial composition that is frozen or dried (e.g., lyophilized).

98. The dosage form of any one of claims 95-97, wherein the dosage form is suitable for sinonasal administration using a syringe, catheter, nasal dropper, e.g., for nasal irrigation, spray, nebulizer, or aerosolizer, optionally a pump aerosol or a pulsating aerosol.

99. The dosage form of any one of claims 95-98, wherein the dosage form is an aqueous solution/liquid suspension (e.g., a saline solution), spray, (dry) powder or aerosol.

100. A composition comprising the substantially complete sinonasal microbial composition of any one of claims 89-94.

101. A medical kit comprising:
a. an applicator configured for delivery of the substantially complete sinonasal microbial composition of any one of claims 89-94 or the composition of claim 100 to the human sinonasal cavities, wherein the composition is optionally a fluid, aerosol, mist or dry powder; and
b. the substantially complete sinonasal microbial composition of any one of claims 89-94 or the composition of claim 100.
